(19) United States
(12) Patent Application Publication
(10) Pub. No.: US 2014/0349852 A1
(43) Pub. Date: Nov. 27, 2014

(54) METABOLIC CONTROL OF SEED GERMINATION

(71) Applicants: Roger Beachy, St. Louis, MO (US); Hiroyuki Nonogaki, St. Louis, MO (US)

(72) Inventors: Roger Beachy, St. Louis, MO (US); Hiroyuki Nonogaki, St. Louis, MO (US)

(73) Assignees: Oregon State University, Corvallis, OR (US); Donald Danforth Plant Science Center, St. Louis, MO (US)

(21) Appl. No.: 13/825,493

(22) PCT Filed: Sep. 21, 2011
(86) PCT No.: PCT/US2011/052593

§ 371 Date: Apr. 4, 2013

Related U.S. Application Data
(60) Provisional application No. 61/385,149, filed on Sep. 21, 2010.

(51) Int. Cl. C12N 15/82

(52) U.S. Cl. CPC C12N 15/8217
USPC 504/346; 800/278; 800/285; 800/298

(57) ABSTRACT

This invention provides Methods of conveniently controlling seed germination in genetically modified plants by the administration of a chemical modulator. The plant comprises a hormone-regulating gene placed under the control of a gene switch.

### CROSS REFERENCE TO RELATED APPLICATIONS

This application claims the benefit of U.S. provisional patent application No. 61/385,149 filed on Sep. 21, 2010 the entire contents of which are incorporated herein by reference.

### BACKGROUND OF THE INVENTION

### Field of the Invention

The present invention relates to germination-control in genetically modified plants.

Seed germination is the emergence of a new plant from seed. A quiescent embryo contained in a dry seed becomes active after hydration, which is called imbibition. The embryo emerges from a seed through the covering tissues such as the testa (seed coat) and the endosperm (nutritive tissue). Initiation of seed germination is a critical decision for plants. Therefore, seeds have evolved strategies to repress seed germination. Seeds sometimes do not germinate even under conditions favourable for germination, which is called seed dormancy.

In agricultural production, deep dormancy is problematic when growers need to germinate seeds and produce seedlings in the fields or greenhouses. In contrast, the lack of seed dormancy in some agricultural crops, which is a consequence of intensive domestication, could cause precocious germination on the maternal plants. For example, pre-harvest sprouting during wheat production drastically reduces grain quality and could result in significant economical losses. Therefore, it is critical to develop technologies to prevent seed germination, as well as those to promote seed germination. Such technologies can also be utilized to protect intellectual properties of specific genetic lines such as value-added transgenic plants.

Seed dormancy and germination are controlled mainly by the balance of abscisic acid (ABA) and gibberellin (GA), two plant hormones. Abscisic acid is a sesquiterpene hormone that maintains seed dormancy and suppresses seed germination while GA is a diterpene hormone that releases seed dormancy and induces seed germination. Endogenous levels of active ABA and GA are determined by the relative rates of biosynthesis and deactivation (conversion into inactive forms) (1).

The key regulatory step of ABA biosynthesis which is catalyzed by 9-cis-epoxycarotenoid dioxygenases (NCEDs) is the cleavage of 9-cis-epoxycarotenoids to produce xanthoxin (2, 3). After the discovery of VP14, a maize NCED (4), many genes encoding this enzyme have been isolated in different agricultural species including tomato (Solanum lycopersicon) (5), potato (Solanum tuberosum) (6), avocado (Persea americana) (7) and orange (Citrus sinensis) (8). In Arabidopsis thaliana (called Arabidopsis hereafter), a model species used for proof-of-concept experiments for this invention, NCED6 and NCED9 are specifically associated with ABA biosynthesis in seeds (9). NCED6 is expressed exclusively in the endosperm of developing Arabidopsis seeds. NCED9 is detected in both the embryo and endosperm (2).

Another step critical for the regulation of the ABA level is the conversion of active ABA to inactive forms. In nondormant Arabidopsis seeds, ABA levels are reduced shortly after the start of imbibition. ABA deactivation plays an important role during this period (10). ABA can be deactivated through hydroxylation or conjugation (3). A cytochrome P450 monooxygenase (ABA 8′-hydroxylase) oxidizes the C-8′ position of the methyl group of active ABA and converts it to a less active form. Arabidopsis CYP707A2 encodes an ABA 8′-hydroxylase which is responsible for the rapid decrease in ABA levels during seed imbibition. cyp707a2 mutant seeds contain six-fold higher ABA levels compared to wild-type seeds and exhibit hyperdormancy (10). Therefore, CYP707A2 is another target to modulate the ABA levels in seeds.

GA is an important antagonist to ABA in terms of seed germination control. There are many different GA molecules in plants, however only a few of them such as GA₁ and GA₄ are active in physiological processes (11). In Arabidopsis seeds, GA₄ is the major active GA. GA₄ is produced from its precursor GA₉ by the action of GA 3-oxidase which is the final reaction and the rate-limiting step in the GA biosynthesis pathway. Major genes encoding this enzyme for seed germination related processes in Arabidopsis are GA3ox1 and GA3ox2 (12). Seeds lacking expression of either of these two genes can still germinate, however, seeds that contain both mutations, designated ga3ox1 ga3ox2 double mutants do not germinate indicating that both genes function in seed germination in a redundant manner (13). Thus, modification of GA3ox1 or GA3ox2 could change seed germination.

### SUMMARY OF THE INVENTION

This invention provides a genetically modified plant comprising a gene under the control of a gene switch; wherein transcription of the gene modulates the level of one or more germination hormones. According to the present invention, germination may be controlled by administration of a chemical modulator of the gene switch.

A genetically modified plant (the “host”) of the present invention comprises (1) a trans-acting factor which is controlled by a chemical modulator; (2) a promoter comprising a cis-element capable of binding the trans-acting factor; and (3) at least one hormone-regulating gene operably linked to the inducible promoter, wherein the level of one or more germination hormones such as abscisic acid (ABA) and/or gibberellin (GA) is modulated upon transcription of the hormone-regulating gene, thereby controlling seed germination.

In one embodiment, the hormone-regulating gene encodes a hormone-metabolizing enzyme. Optionally, the hormone-metabolizing enzyme is an ABA-synthesis enzyme such as a 9-cis-epoxycarotenoid dioxygenase (NCED), for example, NCED6 or NCED9. Optionally, the hormone-metabolizing enzyme is an ABA-catabolism enzyme such as an ABA 8′-hydroxylase. Optionally, the hormone-metabolizing enzyme is a GA-synthesis enzyme such as a GA3 oxidase, for example, GA3ox1 and GA3ox2.

In one embodiment, the transcription factor is an exogenous transcription factor and the plant further comprises a gene encoding the exogenous transcription factor operably linked to a second promoter. Optionally, the exogenous transcription factor comprises an ecdysone receptor (EcR) and the cis-element of the inducible promoter comprises an ecdysone response element (EcRE). Optionally, the inducer is a methoxyfenozide.

In one embodiment, the host comprises both a seed-dormancy transgene and a seed-germination transgene.

In one aspect, the invention provides a mechanism for controlling germination in a plant.

In one aspect, the invention provides a genetically modified plant with controllable germination.

In one aspect, the invention provides constructs for conferring germination control in a photosynthetic organism.

In one aspect, the invention provides a method of controlling germination comprising administering an inducer to a genetically modified plant of the invention. Optionally, said controlling comprises inducing germination. Optionally, said controlling comprises suppressing germination.

### BRIEF DESCRIPTION OF THE DRAWINGS

FIG. 1 depicts a graphical representation of a gene switch system.

FIG. 2 depicts an induction of an ABA metabolism gene (NCED6) in Arabidopsis rosettes (A) and 29 h-imbibed seeds (B) with the ligand. Expression of NCED6 was examined in wild type (WT) and the AGE:NCED6 transgenic plants in the absence (−) or presence (+) of the ligand. Equal loading images ribosomal RNA (rRNA) are shown.

FIG. 3 (A) depicts an example of germination suppression observed in plants transformed with an ABA metabolism gene under the control of a gene switch (AGE:NCED6 lines (5-176 and 15-195). Left, uninduced; Right, induced.). The ligand did not affect wild-type (WT) seed germination. (B) depicts representative images of the AGE:NCED6 seeds arrested immediately after testa rupture (left) or radicle emergence (right) in the presence of the ligand

FIG. 4 depicts an increase in ABA levels specifically in induced transgenic seeds. ABA levels in wild-type (WT) and transgenic (line 15-133) seeds in the absence (−) or presence (+) of the ligand are shown.

FIG. 5 (A) depicts germination suppression in transgenic (AGE:NCED6) seeds induced by the ligand and successful recovery of germination by 10 μM fluridone, a carotenoid biosynthesis (hence ABA biosynthesis) inhibitor. Results of wild type (WT) and transgenic lines (5-175, 8-181 and 15-195) are shown. “−”=Water control; “+”=Intrepid; “F”=fluridone. (B) depicts recovery of germination in the AGE:NCED6 seeds by fluridone (10 μM).

FIG. 6 depicts a schematic representation exemplary of ABA (A) and GA (B) biosynthesis and deactivation pathways. A single arrow does not represent a single reaction. (¦) indicates the site of inhibition in the carotenoid biosynthesis by fluridone. The figures are modified from (2).

FIG. 7 depicts suppression of seed germination induced by the ligand in multiple seed lots of transgenic (AGE:NCED6 homozygous) lines. (−), uninduced; (+), induced with ligand. WT: wild type Col-0.

FIG. 8 depicts suppression of seed germination induced by the ligand in transgenic (AGE:NCED6 homozygous) lines with transparent testa (tt) mutant background. (−), uninduced; (+), induced with ligand. tt3 and tt4: control tt3 and tt4 mutant seeds without AGE:NCED6.

FIG. 9 depicts suppression of seed germination induced by the ligand in multiple seed lots of transgenic (AGE:NCED9 homozygous) lines. (−), uninduced; (+), induced with ligand. WT: wild type Col-0.

FIG. 10 depicts stage specificity of NCED6 expression during Arabidopsis seed development. RNA was extracted from siliques at stage I (green siliques<0.5 cm), II (green siliques>1.0 cm), III (yellow fresh siliques) and IV (brown dry siliques). NCED6 peaked around the stage II. Equal loading images of ribosomal RNA (rRNA) are shown.

FIG. 11 depicts Intrepid®2F dose responses of transgenic (AGE:NCED6) seeds. All above mentioned induction experiments were done using ×10,000 dilution of the commercially available Intrepid®2F solution, which contains approximately 62M of methoxyfenozide as an active ingredient. Further dilutions of the original solution were examined to demonstrate that the concentration of Intrepid®2F applied determines the degree of suppression of germination suppression.

FIG. 12 depicts suppression of precocious germination in siliques of transgenic plants expressing a seed-dormancy gene.

FIG. 13 depicts native expression of NCED6 in Camelina sativa.

FIG. 14 depicts a sequence alignment of various NCEDs from Arabidopsis thaliana.

FIG. 15 depicts an alignment of an NCED6 and NCED9 sequence from Arabidopsis thaliana.

FIG. 16 depicts an alignment of an Arabidopsis and Camelina NCED6 sequences.

FIG. 17 depicts the construction of the AGE:NCED6 and AGE:NCED9 gene constructs.

FIG. 18 depicts the construction of the AGE:CYP707A2 [ABA 8′ hydroxylase].

FIG. 19 depicts the effect of a transgene comprising the NCED6 promoter/NCED6 coding region construct on the germination of Camelina seeds

FIG. 20 depicts photographs showing an example of germination suppression observed transgenic Camelina seeds that contain AGE:NCED6 transgene following treatment with methoxyfenozide.

FIG. 21 depicts ABA levels in Camelina seeds in wild type and transgenic plants that contain the AGE:NCED6 following induction by methoxyfenozide.

FIG. 22 depicts photographs showing an example of Camelina seeds following induction of the AGE:CYP707A2 gene [ABA 8′ hydroxylase] during imbibition of seeds from transgenic plants that carry the gene.

FIG. 23 depicts the percentage of germination seeds of wild type and three transgenic lines that carry AGE:CYP707A2 [ABA 8′ hydroxylase] germinated in the presence or absence of MOF or ABA.

### DETAILED DESCRIPTION OF THE INVENTION

### Definitions

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the terms “cell,” “cells,” “cell line,” “host cell,” and “host cells,” are used interchangeably and, encompass plant, invertebrate, non-mammalian vertebrate, insect, algal, and mammalian cells. All such designations include cell populations and progeny. Thus, the terms “transformants” and “transfectants” include the primary subject cell and cell lines derived therefrom without regard for the number of transfers.

A “chemical modulator” means a chemical inducer or a chemical inhibitor of trans-acting factor (e.g. as part of a gene switch).

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

A “control host” means a host which is substantial identical to a reference host excepting that it has not been transgenically modified according to the present invention.

The term “exemplary” (or “e.g.” or “by example”) means a non-limiting example.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are regulatory sequences of nucleic acids, such as promoters, leaders, enhancers, introns, recognition motifs for RNA, or DNA binding proteins, polyadenylation signals, terminators, internal ribosome entry sites (IRES) and the like, that have the ability to affect the transcription or translation of a coding sequence in a host cell. Exemplary expression control sequences are described in Goeddel; Gene Expression Technology: Methods in Enzymology 185, Academic Press, San Diego, Calif. (1990).

A “gene” is a sequence of nucleotides which code for a functional gene product. Generally, a gene product is a functional protein. However, a gene product can also be another type of molecule in a cell, such as RNA (e.g., a tRNA or an rRNA). A gene may also comprise expression control sequences (i.e., non-coding) sequences as well as coding sequences and introns. The transcribed region of the gene may also include untranslated regions including introns, a 5′-untranslated region (5′-UTR) and a 3′-untranslated region (3′-UTR).

The term “heterologous” refers to a nucleic acid or protein which has been introduced into an organism (such as a plant, animal, or prokaryotic cell), or a nucleic acid molecule (such as chromosome, vector, or nucleic acid construct), which are derived from another source, or which are from the same source, but are located in a different (i.e. non native) context.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention.

To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

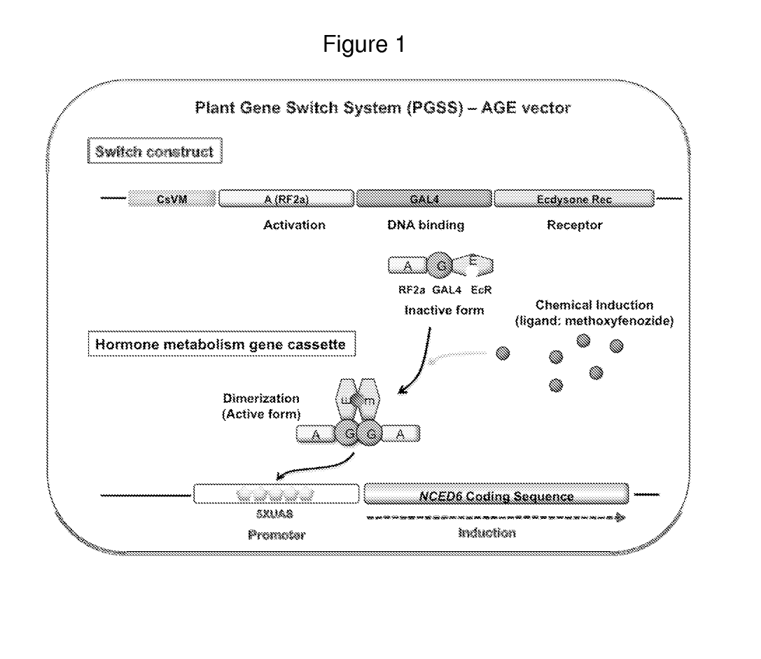

The terms “gene switch”, or “Plant Gene Switch System”, or “PGSS” refers generally to a chemically inducible gene expression system. In some embodiments the gene switch comprises a receptor fusion protein encoding a trans-acting factor that responds to a suitable ligand to activate the gene expression of a second expression cassette via an interaction with the cis acting elements in the second expression cassette. In one embodiment, the first expression cassette comprises expression control sequences that drive the expression of a receptor fusion, which acts as the trans acting factor. In some embodiments the trans acting factor comprises an activation domain operably coupled to a DNA binding protein which is operably coupled to a ligand binding domain of a receptor. In this embodiment, activation of the first expression cassette by the addition of a receptor modulator (ligand) induces the ligand dependent dimerization of the encoded fusion protein which can then bind to expression control sequences (Up Stream Activating Sequences, or “UAS”) of a second expression cassette, thereby leading to the expression of the encoded protein which is operatively coupled to the UAS. An exemplary PGSS, the “AGE” vector system is shown schematically in FIG. 1. In different embodiments, the first and second expression cassettes may be part of a single contiguous genetic construct, or may be separated onto 2 non contiguous genetic constructs.

The term “germination hormone” means a plant hormone that controls the germination state of the plant. A germination hormone may be a hormone that induces seed germination, suppresses seed germination, maintains seed dormancy, or releases seed dormancy.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., 1987, Cell, 50:667). Such proteins (and their encoding nucleic acids) have sequence homology, as reflected by their sequence similarity, whether in terms of percent identity or by the presence of specific residues or motifs and conserved positions.

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

The term “isolated,” when used to describe a protein or nucleic acid, means that the material has been identified and separated and/or recovered from a component of its natural environment. Contaminant components of its natural environment are materials that would typically interfere with research, diagnostic or therapeutic uses for the protein or nucleic acid, and may include enzymes, hormones, and other proteinaceous or non-proteinaceous solutes. In some embodiments, the protein or nucleic acid will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated proteins and nucleic acids will be prepared by at least one purification step.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., (1990), J. Molec. Biol. 215: 403-410 and Altschul et al., (1997), Nuc. Acids Res. 25: 3389-3402).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., (1990), J. Mol. Biol. 215: 403-410). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W. T. and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

“Hormone-regulating gene” means a polynucleotide that, when transcribed or expressed, modulates the activity, expression or spatial distribution of a germination hormone. Examples of hormone-regulating genes include genes that encode enzymes which metabolize a germination hormone (“hormone-metabolizing genes”) and nucleic acid sequences that comprise anti-sense sequences such as RNAi agents (e.g. siRNA) which inhibit the expression of hormone-metabolizing genes.

The terms “operably linked”, “operatively linked,” or “operatively coupled” and synonyms thereof are used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more DNA or RNA nucleotide sequences chosen from: (a) a nucleotide sequence capable of increasing translation; (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The terms “polynucleotide,” “nucleotide sequence” and “nucleic acid” are used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches.

As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), the Figwort Mosaic Virus (FMV) 35S promoter (U.S. Pat. No. 5,378,619), the cassaya vein mosaic virus (U.S. Pat. No. 7,601,885). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art. As used herein, the term “substantially free” is used operationally, in the context of analytical testing of the material. Preferably, purified material substantially free of contaminants is at least 50% pure; more preferably, at least 75% pure, and more preferably still at least 95% pure. Purity can be evaluated by chromatography, gel electrophoresis, immunoassay, composition analysis, biological assay, and other methods known in the art. The term “substantially pure” indicates the highest degree of purity, which can be achieved using conventional purification techniques known in the art.

The term “sequence similarity” refers to the degree of identity or correspondence between nucleic acid or amino acid sequences that may or may not share a common evolutionary origin. However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

In particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

As used herein, a “transgenic plant” is one whose genome has been altered by the incorporation of heterologous genetic material, e.g. by transformation as described herein. The term “transgenic plant” is used to refer to the plant produced from an original transformation event, or progeny from later generations or crosses of a transgenic plant, so long as the progeny contains the heterologous genetic material in its genome.

The term “transformation” or “transfection” refers to the transfer of one or more nucleic acid molecules into a host cell or organism. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction, or infection with viruses or other infectious agents.

“Transformed”, “transduced”, or “transgenic”, in the context of a cell, refers to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells have been through the transformation process and contain foreign nucleic acid. The term “untransformed” refers to cells that have not been through the transformation process.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Buchanan et al., Biochemistry and Molecular Biology of Plants, Courier Companies, USA, 2000; Miki and Iyer, Plant Metabolism, 2^{nd} Ed. D. T. Dennis, D H Turpin, D D Lefebrve, D G Layzell (eds) Addison Wesly, Langgmans Ltd. London (1997); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Overview of the Invention

Surprisingly, it has now been discovered that seed germination can be tightly controlled by placing certain hormone-regulating genes under the control of a gene switch. According to some embodiments of the present invention, a host plant may be transformed with a hormone-regulating gene under the control of a plant gene switch system (PGSS). Transcription of the hormone-regulating gene may then be modulated by administration of a chemical modulator of the gene switch to, for example, induce seed germination or maintain seed dormancy. In some embodiments, the genetic background of the plant is further modified by the over expression of a gene involved in abscisic acid (ABA) synthesis to maintain seed dormancy, until germination is desired through the induced expression of an enzyme involved in ABA deactivation via the gene switch.

### Germination Hormones and Hormone-Regulating Genes

According to the present invention, germination may be controlled by transforming a host plant with one or more hormone-regulating genes which is operably coupled to a gene switch. Useful hormone-regulating genes include genes which modulate the rate of synthesis and/or deactivation (e.g. conversion into inactive forms) of one or more germination hormones upon transcription or expression (sometimes referred to simply as ‘expression’) of the gene. The germination hormone may be any hormone that induces seed germination, suppresses seed germination, maintains seed dormancy, or releases seed dormancy.

Germination Hormones

In one embodiment, the germination hormone is an abscisic acid (ABA) or gibberellin (GA). ABA is a sesquiterpene hormone that maintains seed dormancy and suppresses seed germination while GA is a diterpene hormone that releases seed dormancy and induces seed germination. In such an embodiment, seed germination (and dormancy) may be determined by the balance of ABA and GA.

Germination can also be controlled by modulating the level of other germination hormones such as brassinosteroid, ethylene, cytokinin and jasmonate.

Hormone-Regulating Genes

According to the present invention, the levels of active germination hormones such as ABA and GA may be controlled by modulating the relative rates of synthesis and deactivation of these hormones. A hormone-regulating gene may increase the ratio of ABA:GA levels or other ratio of hormones which induces dormancy (‘seed dormancy gene’) or may decrease the ratio of ABA:GA levels or other ratio of hormones which induces germination (‘seed germination gene’).

The rate of synthesis of a hormone, such as ABA, may be increased by transforming a host plant with, for example, a hormone-regulating gene encoding a hormone-synthesis enzyme (e.g. NCED), or a hormone-regulating gene encoding an RNAi agent which targets a hormone deactivation enzyme (e.g. CYP707 RNAi). In some embodiments such genes are operably coupled to a gene switch to enable the selective expression of the synthesis enzyme. Similarly, the rate of deactivation of a hormone, such as ABA, may be increased by transforming a host plant with, for example, a hormone-regulating gene encoding a hormone-deactivation enzyme of ABA (e.g. ABA 8′ hydroxylase), or a hormone-regulating gene encoding an RNAi agent which targets a hormone-synthesis enzyme of ABA (e.g. NCED RNAi). In some embodiments such genes are operably coupled to a gene switch to enable the selective expression of the synthesis enzyme.

### Exemplary Abscisic-Acid Deactivation Genes

In one embodiment, the hormone-regulating gene encodes an enzyme which catalyzes a step in the deactivation of abscisic-acid (ABA). Such a gene may be useful to release a seed from dormancy or induce germination. In some embodiments, the enzyme is an ABA 8′-hydroxylase. Other useful ABA deactivation enzymes include for example ABA glucosytransferase.

In any of these methods, DNA constructs, and transgenic organisms, the terms “(+)-abscisic acid 8′-hydroxylase” or “ABA 8′-hydroxylase” or “CYP707” refers to all naturally-occurring and synthetic genes encoding an ABA 8′-hydroxylase capable of catalyzing the hydroxylation of abscisic acid according to the following chemical reaction.

abscisic acid→8′-hydroxyabscisic acid

In one aspect the ABA 8′-hydroxylase is from planta. In a further embodiment the ABA 8′-hydroxylase is from Arabidopsis thaliana. Representative species and Gene bank accession numbers for various species of ABA 8′-hydroxylase are listed below in Table D1, and genes from other species may be readily identified by standard homology searching of publicly available databases.

TABLE D1
Exemplary ABA Hydroxylases

| Uniprot | | |
|---|---|---|
| Accession | Name | Organism |
| O81077 | Abscisic acid 8′-hydroxylase 2 | Arabidopsis thaliana (Mouse-ear cress) |
| D7LKC0 | CYP707A2 | Arabidopsis lyrata subsp. lyrata |
| B2BXN2 | Cytp450-1 | Cleome spinosa |
| B9HPE8 | Cytochrome P450 | Populus trichocarpa (Western balsam poplar) |
| | | (Populus balsamifera subsp. trichocarpa) |
| B9GJA3 | Predicted protein | Populus trichocarpa (Western balsam poplar) |
| | | (Populus balsamifera subsp. trichocarpa) |
| B1B1U2 | ABA 8-oxidase | Lactuca sativa (Garden lettuce) |
| D7SKC6 | Whole genome shotgun sequence | Vitis vinifera (Grape) |
| | of line PN40024 . . . | |
| D7TFC7 | Whole genome shotgun sequence | Vitis vinifera (Grape) |
| | of line PN40024 . . . | |
| B9RY37 | Cytochrome P450, putative | Ricinus communis (Castor bean) |
| Q9FH76 | Abscisic acid 8′-hydroxylase 3 | Arabidopsis thaliana (Mouse-ear cress) |
| B9N0P8 | Predicted protein | Populus trichocarpa (Western balsam poplar) |
| | | (Populus balsamifera subsp. trichocarpa) |
| Q949P1 | Abscisic acid 8′-hydroxylase 1 | Arabidopsis thaliana (Mouse-ear cress) |
| Q3HNF4 | ABA 8′-hydroxylase CYP707A1 | Solanum tuberosum (Potato) |
| D7MU38 | Predicted protein | Arabidopsis lyrata subsp. lyrata |
| B9N4Y2 | Cytochrome P450 | Populus trichocarpa (Western balsam poplar) |
| | | (Populus balsamifera subsp. trichocarpa) |
| B1B1T9 | ABA 8-oxidase | Lactuca sativa (Garden lettuce) |
| B9SC56 | Cytochrome P450, putative | Ricinus communis (Castor bean) |
| D7TPI4 | Whole genome shotgun sequence | Vitis vinifera (Grape) |
| | of line PN40024 . . . | |
| B1B1U1 | ABA 8-oxidase | Lactuca sativa (Garden lettuce) |
| A5CAN8 | Putative uncharacterized protein | Vitis vinifera (Grape) |
| B9HS10 | Predicted protein | Populus trichocarpa (Western balsam poplar) |
| | | (Populus balsamifera subsp. trichocarpa) |
| Q05JG2 | Abscisic acid 8′-hydroxylase 1 | Oryza sativa subsp. japonica (Rice) |
| Q09J79 | Abscisic acid 8′-hydroxylase 1 | Oryza sativa subsp. indica (Rice) |
| Q0H212 | Abscisic acid 8′-hydroxylase | Phaseolus vulgaris (Kidney bean) (French |
| | | bean) |
| C0PID5 | Putative uncharacterized protein | Zea mays (Maize) |
| Q09J78 | Abscisic acid 8′-hydroxylase 2 | Oryza sativa subsp. indica (Rice) |
| C9K222 | ABA 8-hydroxylase | Triticum monococcum (Einkorn wheat) |
| | | (Small spelt) |
| B9RYI0 | Cytochrome P450, putative | Ricinus communis (Castor bean) |
| B8BBL9 | Putative uncharacterized protein | Oryza sativa subsp. indica (Rice) |
| Q6ZDE3 | Abscisic acid 8′-hydroxylase 2 | Oryza sativa subsp. japonica (Rice) |
| B9GNW1 | Cytochrome P450 | Populus trichocarpa (Western balsam poplar) |
| | | (Populus balsamifera subsp. trichocarpa) |
| B9R7C1 | Cytochrome P450, putative | Ricinus communis (Castor bean) |
| C0P6C1 | Putative uncharacterized protein | Zea mays (Maize) |
| D7SVY2 | Whole genome shotgun sequence | Vitis vinifera (Grape) |
| | of line PN40024 . . . | |
| C5YMA6 | Putative uncharacterized protein | Sorghum bicolor (Sorghum) |
| | Sb07g022990 | (Sorghum vulgare) |
| C7DTJ6 | Cytochrome P450 family 707 | Lepidium sativum (Garden cress) |
| | subfamily A polype . . . | |
| C0HDV2 | Putative uncharacterized protein | Zea mays (Maize) |
| Q0H210 | Abscisic acid 8′-hydroxylase | Phaseolus vulgaris (Kidney bean) (French |
| | | bean) |
| A5AVZ8 | Putative uncharacterized protein | Vitis vinifera (Grape) |
| Q0H211 | Abscisic acid 8′-hydroxylase | Phaseolus vulgaris (Kidney bean) (French |
| | | bean) |

It is well established that the genetic code is degenerate and that some amino acids have multiple codons, and accordingly, multiple polynucleotides can encode the ABA 8′-hydroxylase of the invention. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode ABA 8′-hydroxylase proteins can be obtained from by “back-translation” (for example by using Computer programs such as “BackTranslate” (GCG™ Package, Acclerys, Inc. San Diego, Calif.) of the deduced coding sequences derived from ABA 8′-hydroxylase genomic clones, from cDNA or EST sequences, or any of the sequences listed in Table D1.

Examples of nucleic acids that contain mature ABA 8′-hydroxylase protein-encoding nucleotide sequences include but are not limited to a sequence with at least 70%, at least 80%, at least 85%, at least 90%, at least 95%, at least 97%, at least 99%, or 100% sequence identity to SEQ. ID. No. 3.

In some embodiments, the non-native ABA 8′-hydroxylase-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the non-native ABA 8′-hydroxylase-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the non-native ABA 8′-hydroxylase-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the ABA 8′-hydroxylase polynucleotide sequences listed in Table D1. Polynucleotide “variants” may contain one or more substitutions, additions, deletions and/or insertions in relation to a reference polynucleotide. Generally, variants of the ABA 8′-hydroxylase reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence (i.e. to SEQ. ID, No. 3) as determined by sequence alignment programs described elsewhere herein using default parameters.

In some embodiments the ABA 8′-hydroxylase which may be used in any of the methods and plants of the invention may have amino acid sequences which are substantially homologous, or substantially similar to any of the native ABA 8′-hydroxylase amino acid sequences, for example, to any of the native ABA 8′-hydroxylase amino acid sequences encoded by the genes listed in Table D1.

For use in the present invention, the ABA 8′-hydroxylase may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of an ABA 8′-hydroxylase, are also specifically included in any of the methods of the invention including for example, pyroglutamyl, iso-aspartyl, proteolytic, phosphorylated, glycosylated, reduced, oxidatized, isomerized, and deaminated variants of the ABA 8′-hydroxylase.

Alternatively, the ABA 8′-hydroxylase may have an amino acid sequence having at least 30% preferably at least 40, 50, 60, 70, 75, 80, 85, 90, 95, 98, or 99% identity with an ABA 8′-hydroxylase encoded by a gene listed in Table D1. In a preferred embodiment, the ABA 8′-hydroxylase for use in any of the methods and plants of the present invention is at least 80% identical to the mature ABA 8′-hydroxylase from Arabidopsis thaliana (SEQ. ID. NO. 7) below.

(SEQ. ID. NO. 7)
MQISSSSSSNFFSSLYADEPALITLTIVVVVVVLLFKWWLHWKEQRLR
LPPGSMGLPYIGETLRLYTENPNSFFATRQNKYGDIFKTHILGCPCVM
ISSPEAARMVLVSKAHLFKPTYPPSKERMIGPEALFFHQGPYHSTLKR
LVQSSFMPSALRPTVSHIELLVLQTLSSWTSQKSINTLEYMKRYAFDV
AIMSAFGDKEEPTTIDVIKLLYQRLERGYNSMPLDLPGTLFHKSMKAR
IELSEELRKVIEKRRENGREEGGLLGVLLGAKDQKRNGLSDSQIADNI
IGVIFAATDTTASVLTWLLKYLHDHPNLLQEVSREQFSIRQKIKKENR
RISWEDTRKMPLTTRVIQETLRAASVLSFTFREAVQDVEYDGYLIPKG
WKVLPLFRRIHHSSEFFPDPEKFDPSRFEVAPKPYTYMPFGNGVHSCP
GSELAKLEMLILLHHLTTSFRWEVIGDEEGIQYGPFPVPKKGLPIRVT
PI.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations can be introduced into ABA 8′-hydroxylase and are considered within the scope of the invention. Mutations of ABA 8′-hydroxylase that increase the activity of the protein are known and may be used in the methods and plants of the invention. The ABA 8′-hydroxylase may thus include one or more amino acid deletions, additions, insertions, and/or substitutions based on any of the naturally-occurring isoforms of ABA 8′-hydroxylase. These may be contiguous or non-contiguous. Representative variants may include those having 1 to 8, or more preferably 1 to 4, 1 to 3, or 1 or 2 amino acid substitutions, insertions, and/or deletions as compared to any of sequences listed in Table D1.

The variants, derivatives, and fusion proteins of ABA 8′-hydroxylase are functionally equivalent in that they have detectable ABA 8′-hydroxylase activity. More particularly, they exhibit at least 5%, at least 10%, at least 20%, at least 30%, at least 40%, preferably at least 60%, more preferably at least 80% of the activity of ABA 8′-hydroxylase from Arabidopsis thaliana SEQ. ID. NO. 7, and are thus they are capable of substituting for ABA 8′-hydroxylase itself.

All such variants, derivatives, fusion proteins, or fragments of ABA 8′-hydroxylase are included, and may be used in any of the polynucleotides, vectors, host cell and methods disclosed and/or claimed herein, and are subsumed under the term “ABA 8′-hydroxylase”. Suitable assays for determining functional ABA 8′-hydroxylase activity are well known in the art.

### Exemplary Abscisic-Acid Synthesis Genes

In one embodiment, the hormone-regulating gene encodes an enzyme which catalyzes a step in the synthesis of abscisic-acid (ABA). Such a gene may be useful to maintain dormancy or prevent germination. In some embodiments, the enzyme is a 9-cis-epoxycarotenoid dioxygenase (NCED), which catalyzes the first step of abscisic-acid biosynthesis from carotenoids in chloroplasts. Other useful ABA synthesis enzymes include, for example, ABA aldehyde oxidase (AAO).

In any of these methods, DNA constructs, and transgenic organisms, the terms “9-cis-epoxycarotenoid dioxygenase” or “NCED” refers to all naturally-occurring and synthetic genes encoding 9-cis-epoxycarotenoid dioxygenase capable of oxidizing 9-cis-epoxycarotenoid, 9-cis-violaxanthin, or 9′-cis-neoxanthin to form 2-cis,4-trans-xanthoxin.

In one aspect the 9-cis-epoxycarotenoid dioxygenase is from planta. In a further embodiment the 9-cis-epoxycarotenoid dioxygenase is from Arabidopsis thaliana. In one embodiment, the 9-cis-epoxycarotenoid dioxygenase is a seed-specific NCED.

Optionally, the 9-cis-epoxycarotenoid dioxygenase is an NCED1, NCED2, NCED3, NCED4, NCED5, NCED6, NCED7, NCED8, or NCED9. Representative species and Gene bank accession numbers for various species of 9-cis-epoxycarotenoid dioxygenase are listed below in Table D2.

TABLE D2
Exemplary 9-cis-epoxycarotenoid dioxygenase (NCED) genes

| UniProt | | |
|---|---|---|
| Accession | Name | Organism |
| Q9LRM7 | 9-cis-epoxycarotenoid dioxygenase | Arabidopsis thaliana (Mouse-ear cress) |
| | NCED6, chlo . . . | |
| D7L472 | Nine-cis-epoxycarotenoid dioxygenase 6 | Arabidopsis lyrata subsp. lyrata |
| D5L702 | 9-cis-epoxycarotenoid dioxygenase | Camelina sativa (False flax) (Gold-of- |
| | | pleasure) |
| B9RWM0 | 9-cis-epoxycarotenoid dioxygenase, | Ricinus communis (Castor bean) |
| | putative | |
| D7TS80 | Whole genome shotgun sequence of line | Vitis vinifera (Grape) |
| | PN40024 . . . | |
| Q2PHF9 | 9-cis-epoxycarotenoid dioxygenase 4 | Lactuca sativa (Garden lettuce) |
| D7MW32 | Nine-cis-epoxycarotenoid dioxygenase 9 | Arabidopsis lyrata subsp. lyrata |
| C3VEQ3 | 9-cis-epoxycarotenoid dioxygenase | Oncidium Gower Ramsey |
| Q2VEX0 | Putative 9-cis epoxycarotenoid | Daucus carota subsp. Sativus |
| | dioxygenase | |
| D7L349 | Nine-cis-epoxycarotenoid dioxygenase3 | Arabidopsis lyrata subsp. lyrata |
| Q9M9F5 | 9-cis-epoxycarotenoid dioxygenase | Arabidopsis thaliana (Mouse-ear cress) |
| | NCED9, chlo . . . | |
| Q9LRR7 | 9-cis-epoxycarotenoid dioxygenase | Arabidopsis thaliana (Mouse-ear cress) |
| | NCED3, chlo . . . | |
| C8CEB0 | 9-cis-epoxycarotenoid dioxygenase | Caragana korshinskii |
| Q2VEW8 | Putative 9-cis epoxycarotenoid | Daucus carota subsp. Sativus |
| | dioxygenase | |
| Q9FS24 | Neoxanthin cleavage enzyme | Vigna unguiculata (Cowpea) |
| Q6DLW4 | 9-cis-epoxy-carotenoid dioxygenase 1 | Solanum tuberosum (Potato) |
| O24023 | 9-cis-epoxycarotenoid dioxygenase | Solanum lycopersicum (Tomato) |
| | | (Lycopersicon esculentum) |
| Q0H900 | 9-cis-epoxycarotenoid dioxygenase 3 | Coffea canephora (Robusta coffee) |
| A0ZSY4 | 9-cis-epoxycarotenoid dioxygenase | Raphanus sativus (Radish) |
| Q9AXZ4 | 9-cis-epoxycarotenoid dioxygenase | Persea americana (Avocado) |
| Q8LP16 | Nine-cis-epoxycarotenoid dioxygenase2 | Pisum sativum (Garden pea) |
| B9SU59 | 9-cis-epoxycarotenoid dioxygenase, | Ricinus communis (Castor bean) |
| | putative | |
| Q9M3Z9 | Putative 9-cis-epoxycarotenoid | Solanum tuberosum (Potato) |
| | dioxygenase | |
| C4MK80 | 9-cis-epoxycarotenoid dioxygenase | Malus hupehensis var. mengshanensis |
| Q9M6E8 | 9-cis-epoxycarotenoid dioxygenase | Phaseolus vulgaris (Kidney bean) |
| | NCED1, chlo . . . | (French bean) |
| Q460X8 | 9-cis-epoxycarotenoid dioxygenase | Stylosanthes guianensis |
| Q70KY0 | 9-cis-epoxycarotenoid dioxygenase | Arachis hypogaea (Peanut) |
| Q2PHG2 | 9-cis-epoxycarotenoid dioxygenase 1 | Lactuca sativa (Garden lettuce) |
| Q5URR0 | 9-cis-epoxycarotenoid dioxygenase | Brassica rapa subsp. pekinensis (Chinese |
| | | cabbage) |
| B9S0Z6 | 9-cis-epoxycarotenoid dioxygenase, | Ricinus communis (Castor bean) |
| | putative | |
| A1KXV4 | 9-cis-epoxycarotenoid dioxygenase | Gentiana lutea (Yellow gentian) |
| D0E0F1 | NCED3 | Lilium formosanum |
| A5BR72 | Putative uncharacterized protein | Vitis vinifera (Grape) |
| Q5SGD1 | 9-cis-epoxycarotenoid dioxygenase 1 | Vitis vinifera (Grape) |
| A0SE37 | 9-cis-epoxycarotenoid dioxygenase 3 | Citrus clementina |
| D0E2Y3 | Plastid NCED3 | Lilium hybrid cultivar |
| B9I1W0 | Predicted protein | Populus trichocarpa (Western balsam |
| | | poplar) (Populus balsamifera subsp. |
| | | trichocarpa) |
| Q0PD07 | Putative 9-cis-epoxycarotenoid | Hordeum vulgare (Barley) |
| | dioxygenase | |
| D0E0F2 | NCED3 | Lilium speciosum |
| Q0EF28 | Putative 9-cis-epoxycarotenoid | Cryptomeria japonica (Japanese cedar) |
| | dioxygenase | |
| Q5MBR5 | 9-cis-epoxycarotenoid dioxygenase 3 | Oryza sativa subsp. japonica (Rice) |
| Q0EF33 | Putative 9-cis-epoxycarotenoid | Cryptomeria japonica (Japanese cedar) |
| | dioxygenase | |
| A0JBX8 | Putative 9-cis-epoxycarotenoid | Chrysanthemum morifolium (Florist's |
| | dioxygenase | daisy) (Dendranthema grandiflorum) |
| B9N649 | Predicted protein | Populus trichocarpa (Western balsam |
| | | poplar) (Populus balsamifera subsp. |
| | | trichocarpa) |
| A1KXV3 | 9-cis-epoxycarotenoid dioxygenase | Gentiana lutea (Yellow gentian) |
| Q0EF51 | Putative 9-cis-epoxycarotenoid | Cryptomeria japonica (Japanese cedar) |
| | dioxygenase | |
| Q0EF54 | Putative 9-cis-epoxycarotenoid | Cryptomeria japonica (Japanese cedar) |
| | dioxygenase | |
| Q0EF37 | Putative 9-cis-epoxycarotenoid | Cryptomeria japonica (Japanese cedar) |
| | dioxygenase | |
| Q0EF30 | Putative 9-cis-epoxycarotenoid | Cryptomeria japonica (Japanese cedar) |
| | dioxygenase | |
| Q0EF16 | Putative 9-cis-epoxycarotenoid | Cryptomeria japonica (Japanese cedar) |
| | dioxygenase | |
| Q0EF14 | Putative 9-cis-epoxycarotenoid | Cryptomeria japonica (Japanese cedar) |
| | dioxygenase | |
| Q5MBR3 | 9-cis-epoxycarotenoid dioxygenase 5 | Oryza sativa subsp. japonica (Rice) |
| Q2VEW9 | Putative 9-cis epoxycarotenoid | Daucus carota subsp. Sativus |
| | dioxygenase | |
| Q0EEH1 | Putative 9-cis-epoxycarotenoid | Taxodium distichum (Bald cypress) |
| | dioxygenase | |
| A0SE34 | 9-cis-epoxycarotenoid dioxygenase 5 | Citrus clementina |
| A2XK33 | Putative uncharacterized protein | Oryza sativa subsp. indica (Rice) |
| Q6DLW3 | 9-cis-epoxy-carotenoid dioxygenase 2 | Solanum tuberosum (Potato) |
| A2ZMS7 | Putative uncharacterized protein | Oryza sativa subsp. indica (Rice) |
| D6QXI4 | 9-cis-epoxycarotenoid dioxygenase | Gossypium hirsutum (Upland cotton) |
| | | (Gossypium mexicanum) |
| Q1G7I5 | 9-cis-epoxycarotenoid dioxygenase 1 | Citrus sinensis (Sweet orange) |
| Q0PD06 | Putative 9-cis-epoxycarotenoid | Hordeum vulgare (Barley) |
| | dioxygenase | |
| A5BEU2 | Putative uncharacterized protein | Vitis vinifera (Grape) |
| A0JBX6 | Putative 9-cis-epoxycarotenoid | Chrysanthemum morifolium (Florist's |
| | dioxygenase | daisy) (Dendranthema grandiflorum) |
| Q2PHG0 | 9-cis-epoxycarotenoid dioxygenase 3 | Lactuca sativa (Garden lettuce) |
| O24592 | 9-cis-epoxycarotenoid dioxygenase 1, | Zea mays (Maize) |
| | chloropl . . . | |
| Q1G7I4 | 9-cis-epoxycarotenoid dioxygenase 2 | Citrus sinensis (Sweet orange) |
| Q5SGD0 | 9-cis-epoxycarotenoid dioxygenase 2 | Vitis vinifera (Grape) |
| C5WR66 | Putative uncharacterized protein | Sorghum bicolor (Sorghum) |
| | Sb01g013520 | (Sorghum vulgare) |
| Q285R7 | 9-cis-epoxycarotenoid dioxygenase 1 | Hordeum vulgare var. distichum (Two- |
| | | rowed barley) |
| Q3KRR2 | Putative 9-cis-epoxycarotenoid | Cuscuta reflexa (Southern Asian dodder) |
| | dioxygenase 2 | |
| Q285R6 | 9-cis-epoxycarotenoid dioxygenase 2 | Hordeum vulgare var. distichum (Two- |
| | | rowed barley) |
| Q69NX5 | 9-cis-epoxycarotenoid dioxygenase 4 | Oryza sativa subsp. japonica (Rice) |
| Q0D8J6 | Os07g0154100 protein | Oryza sativa subsp. japonica (Rice) |
| B7SNW4 | Plastid 9-cis-epoxycarotenoid | Crocus sativus (Saffron) |
| | dioxygenase | |
| B6SSJ7 | Viviparous-14 | Zea mays (Maize) |
| B6SV18 | Viviparous-14 | Zea mays (Maize) |
| Q1XHJ6 | 9-cis-epoxycarotenoid-dioxygenase | Citrus unshiu (Satsuma mandarin) |
| | | (Citrus nobilis var. unshiu) |
| Q1XHJ5 | 9-cis-epoxycarotenoid dioxygenase | Citrus sinensis (Sweet orange) |
| Q3KRR3 | 9-cis-epoxycarotenoid dioxygenase 1 | Cuscuta reflexa (Southern Asian dodder) |
| Q70SW7 | Nine-cis-epoxycarotenoid dioxygenase | Bixa orellana (Lipstick tree) |
| Q1XHJ4 | 9-cis-epoxycarotenoid dioxygenase | Citrus limon (Lemon) |
| D7MCT9 | Nine-cis-epoxycarotenoid dioxygenase 2 | Arabidopsis lyrata subsp. lyrata |
| Q2PHG1 | 9-cis-epoxycarotenoid dioxygenase 2 | Lactuca sativa (Garden lettuce) |
| Q8LP15 | Nine-cis-epoxycarotenoid dioxygenase3 | Pisum sativum (Garden pea) |
| Q9AXZ3 | 9-cis-epoxycarotenoid dioxygenase | Persea americana (Avocado) |
| O49505 | 9-cis-epoxycarotenoid dioxygenase | Arabidopsis thaliana (Mouse-ear cress) |
| | NCED2, chlo . . . | |
| C5X9L8 | Putative uncharacterized protein | Sorghum bicolor (Sorghum) |
| | Sb02g003230 | (Sorghum vulgare) |
| Q9C6Z1 | Probable 9-cis-epoxycarotenoid | Arabidopsis thaliana (Mouse-ear cress) |
| | dioxygenase NC . . . | |
| Q3KRR4 | 9-cis-epoxycarotenoid dioxygenase 2 | Phaseolus vulgaris (Kidney bean) |
| | | (French bean) |
| Q1XHJ3 | 9-cis-epoxycarotenoid dioxygenase | Citrus unshiu (Satsuma mandarin) |
| | | (Citrus nobilis var. unshiu) |
| Q1XHJ1 | 9-cis-epoxycarotenoid dioxygenase | Citrus limon (Lemon) |
| Q1XHJ2 | 9-cis-epoxycarotenoid dioxygenase | Citrus sinensis (Sweet orange) |
| D7KEQ5 | Nine-cis-epoxycarotenoid dioxygenase 5 | Arabidopsis lyrata subsp. lyrata |
| B9HYV5 | Predicted protein | Populus trichocarpa (Western balsam |
| | | poplar) (Populus balsamifera subsp. |
| | | trichocarpa) |
| D5G3I2 | 9-cis epoxycarotenoid dioxygenase | Citrullus lanatus (Watermelon) |
| | | (Citrullus vulgaris) |
| A9U162 | Predicted protein | Physcomitrella patens subsp. patens |
| Q6Q2E3 | Putative 9-cis-epoxycarotenoid | Chorispora bungeana (Blue mustard) |
| | dioxygenase | (Chorispora exscapa) |
| A3CJH5 | Putative uncharacterized protein | Oryza sativa subsp. japonica (Rice) |
| B9H081 | Predicted protein | Populus trichocarpa (Western balsam |
| | | poplar) (Populus balsamifera subsp. |
| | | trichocarpa) |
| A9SI61 | Predicted protein | Physcomitrella patens subsp. patens |
| A2YIA1 | Putative uncharacterized protein | Oryza sativa subsp. indica (Rice) |
| C4J3B4 | Putative uncharacterized protein | Zea mays (Maize) |
| D7TQZ9 | Whole genome shotgun sequence of line | Vitis vinifera (Grape) |
| | PN40024 . . . | |
| D7MSS4 | Putative uncharacterized protein | Arabidopsis lyrata subsp. lyrata |
| Q06Z34 | Neoxanthin epoxy-carotenoid cleavage | Coffea canephora (Robusta coffee) |
| | dioxygen . . . | |
| C0HIM3 | Putative uncharacterized protein | Zea mays (Maize) |
| B0FLM8 | Carotenoid cleavage dioxygenase 4 | Rosa damascena (Damask rose) |
| Q52QS6 | 9-cis epoxycarotenoid dioxygenase | Oncidium Gower Ramsey |
| B2Y6C2 | 9-cis-epoxycarotenoid dioxygenase | Cucumis sativus (Cucumber) |
| | NCED2t | |
| B0FLM9 | Carotenoid cleavage dioxygenase 4 | Osmanthus fragrans |
| A0SE36 | Carotenoid cleavage dioxygenase 4b | Citrus clementina |
| A9NV62 | Putative uncharacterized protein | Picea sitchensis (Sitka spruce) |
| B8LMX1 | Putative uncharacterized protein | Picea sitchensis (Sitka spruce) |
| Q0H901 | Carotenoid cleavage dioxygenase 1 | Coffea canephora (Robusta coffee) |
| B9IQS5 | Predicted protein | Populus trichocarpa (Western balsam |
| | | poplar) (Populus balsamifera subsp. |
| | | trichocarpa) |
| Q52QS7 | 9-cis epoxycarotenoid dioxygenase | Oncidium Gower Ramsey |
| A8J6V6 | 9-cis-epoxycarotenoid dioxygenase | Citrus hybrid cultivar |
| Q0H8Z7 | Carotenoid cleavage dioxygenase 1 | Coffea arabica (Coffee) |
| C6ZLC9 | 9-cis-epoxycarotenoid dioxygenase 3 | Musa acuminata AAA Group |
| C6ZLC7 | 9-cis-epoxycarotenoid dioxygenase 1 | Diospyros kaki (Kaki persimmon) |
| | | (Diospyros chinensis) |
| C3TX77 | Carotenoid cleavage dioxygenase | Brachypodium sylvaticum (False brome) |
| B9HQJ0 | Predicted protein | Populus trichocarpa (Western balsam |
| | | poplar) (Populus balsamifera subsp. |
| | | trichocarpa) |
| D2DJ30 | 9-cis-epoxycarotenoid dioxygenase 1t | Prunus avium (Cherry) |
| A9XSC1 | NCED1 protein | Prunus persica (Peach) |
| C3TX78 | Carotenoid cleavage dioxygenase | Brachypodium sylvaticum (False brome) |
| Q2QLJ0 | 9,10-9′,10′ carotenoid cleavage | Oryza sativa subsp. japonica (Rice) |
| | dioxygenase 1 . . . | |
| C5WYW0 | Putative uncharacterized protein | Sorghum bicolor (Sorghum) |
| | Sb01g047540 | (Sorghum vulgare) |
| B9HQJ1 | Predicted protein | Populus trichocarpa (Western balsam |
| | | poplar) (Populus balsamifera subsp. |
| | | trichocarpa) |
| A9PIS5 | Putative uncharacterized protein | Populus trichocarpa × Populus deltoides |
| A0SE35 | Carotenoid cleavage dioxygenase 4a | Citrus clementina |
| B9P4Z1 | Predicted protein | Populus trichocarpa (Western balsam |
| | | poplar) (Populus balsamifera subsp. |
| | | trichocarpa) |
| Q3I0M3 | 9-cis-epoxycarotenoid dioxygenase 7 | Rumex palustris |
| A5ASS8 | Putative uncharacterized protein | Vitis vinifera (Grape) |
| Q2VEW7 | Putative carotenoid cleavage | Daucus carota subsp. Sativus |
| | dioxygenase | |
| Q0ILK1 | Os12g0640600 protein | Oryza sativa subsp. japonica (Rice) |
| B5ARZ8 | Carotenoid cleavage dioxygenases | Malus hupehensis var. mengshanensis |
| B4FBA4 | 9,10-9,10 carotenoid cleavage | Zea mays (Maize) |
| | dioxygenase 1 | |
| A0SMH9 | Carotenoid cleavage dioxyganase 1 | Zea mays (Maize) |
| Q5U905 | Carotenoid cleavage dioxygenase | Zea mays (Maize) |
| Q45VT7 | 9,10-9′,10′ carotenoid cleavage | Zea mays (Maize) |
| | dioxygenase 1 | |
| B9H4M8 | Predicted protein | Populus trichocarpa (Western balsam |
| | | poplar) (Populus balsamifera subsp. |
| | | trichocarpa) |
| A9RJA4 | Predicted protein | Physcomitrella patens subsp. patens |
| B8BN80 | Putative uncharacterized protein | Oryza sativa subsp. indica (Rice) |
| O49675 | Probable carotenoid cleavage | Arabidopsis thaliana (Mouse-ear cress) |
| | dioxygenase 4, c . . . | |
| A0JBX5 | Carotenoid cleavage dioxygenase | Chrysanthemum morifolium (Florist's |
| | | daisy) (Dendranthema grandiflorum) |
| B9S1S9 | 9-cis-epoxycarotenoid dioxygenase, | Ricinus communis (Castor bean) |
| | putative | |
| Q6E4P3 | Carotenoid cleavage dioxygenase 1 | Petunia hybrida (Petunia) |
| A9XSC2 | NCED1 protein | Vitis labrusca × Vitis vinifera |
| Q84KG4 | Dioxygenase | Capsicum annuum (Bell pepper) |
| C6ZJY5 | 9-cis-epoxycarotenoid dioxygenase 2 | Prunus persica (Peach) |
| A0JBX7 | Putative carotenoid cleavage | Chrysanthemum morifolium (Florist's |
| | dioxygenase | daisy) (Dendranthema grandiflorum) |
| Q6E4P5 | Carotenoid cleavage dioxygenase 1A | Solanum lycopersicum (Tomato) |
| | | (Lycopersicon esculentum) |
| Q0H394 | Carotenoid cleavage dioxygenase | Cucumis melo (Muskmelon) |
| D4QE74 | Carotenoid cleavage dioxygenase 1 | Osmanthus fragrans |
| Q84KG5 | Carotenoid 9,10(9′,10′)-cleavage | Crocus sativus (Saffron) |
| | dioxygenase | |
| Q6E4P4 | Carotenoid cleavage dioxygenase 1B | Solanum lycopersicum (Tomato) |
| | | (Lycopersicon esculentum) |
| A9NUZ5 | Putative uncharacterized protein | Picea sitchensis (Sitka spruce) |
| Q6A4I5 | Carotenoid cleavage oxygenase | Solanum lycopersicum (Tomato) |
| | | (Lycopersicon esculentum) |
| D2DJ28 | 9-cis-epoxycarotenoid dioxygenase 1t | Fragaria ananassa (Strawberry) |
| A9Z0V7 | Carotenoid cleavage dioxygenase 1 | Rosa damascena (Damask rose) |
| Q2PHF8 | Carotenoid cleavage dioxygenase 1 | Lactuca sativa (Garden lettuce) |
| B4XWB6 | Fruit-specific 9-cis-epoxycarotenoid | Cucumis melo (Muskmelon) |
| | dioxygen . . . | |
| B2Y6C3 | 9-cis-epoxycarotenoid dioxygenase | Cucurbita moschata (Winter crookneck |
| | DNCED1t | squash) (Cucurbita pepo var. moschata) |
| D7NMX0 | 9-cis-epoxycarotenoid dioxygenase 3t | Solanum lycopersicum (Tomato) |
| | | (Lycopersicon esculentum) |
| D2DJ31 | 9-cis-epoxycarotenoid dioxygenase 2t | Prunus avium (Cherry) |
| C6ZJY4 | 9-cis-epoxycarotenoid dioxygenase 1 | Pyrus × bretschneideri |
| Q2QLI9 | 9,10-9′,10′ carotenoid cleavage | Oryza sativa subsp. japonica (Rice) |
| | dioxygenase 1 . . . | |
| D7MGU3 | Nine-cis-epoxycarotenoid dioxygenase 4 | Arabidopsis lyrata subsp. lyrata |
| A4URT6 | 9-cis-epoxycarotenoid dioxygenase | Castanea mollissima (Chinese chestnut) |
| Q94IR2 | Carotenoid 9,10(9′,10′)-cleavage | Phaseolus vulgaris (Kidney bean) |
| | dioxygenase . . . | (French bean) |
| Q4ZJB4 | Carotenoid cleavage dioxygenase | Suaeda salsa (Seepweed) |
| | | (Chenopodium salsum) |
| Q3T4H1 | 9,10[9′,10′]carotenoid cleavage | Vitis vinifera (Grape) |
| | dioxygenase | |
| B9HQI8 | Predicted protein | Populus trichocarpa (Western balsam |
| | | poplar) (Populus balsamifera subsp. |
| | | trichocarpa) |
| C5H805 | 9-cis-epoxycarotenoid dioxygenase 2 | Solanum lycopersicum (Tomato) |
| | | (Lycopersicon esculentum) |
| Q52QS5 | 9-cis epoxycarotenoid dioxygenase | Oncidium Gower Ramsey |
| D7T313 | Whole genome shotgun sequence of line | Vitis vinifera (Grape) |
| | PN40024 . . . | |
| A9PFV7 | Putative uncharacterized protein | Populus trichocarpa (Western balsam |
| | | poplar) (Populus balsamifera subsp. |
| | | trichocarpa) |
| Q8LP17 | Carotenoid 9,10(9′,10′)-cleavage | Pisum sativum (Garden pea) |
| | dioxygenase . . . | |
| B0FLM7 | Carotenoid cleavage dioxygenase 4a | Chrysanthemum morifolium (Florist's |
| | | daisy) (Dendranthema grandiflorum) |
| Q2PHF7 | Carotenoid cleavage dioxygenase 2 | Lactuca sativa (Garden lettuce) |
| B5BLW2 | Carotenoid cleavage dioxygenase 1 | Medicago truncatula (Barrel medic) |

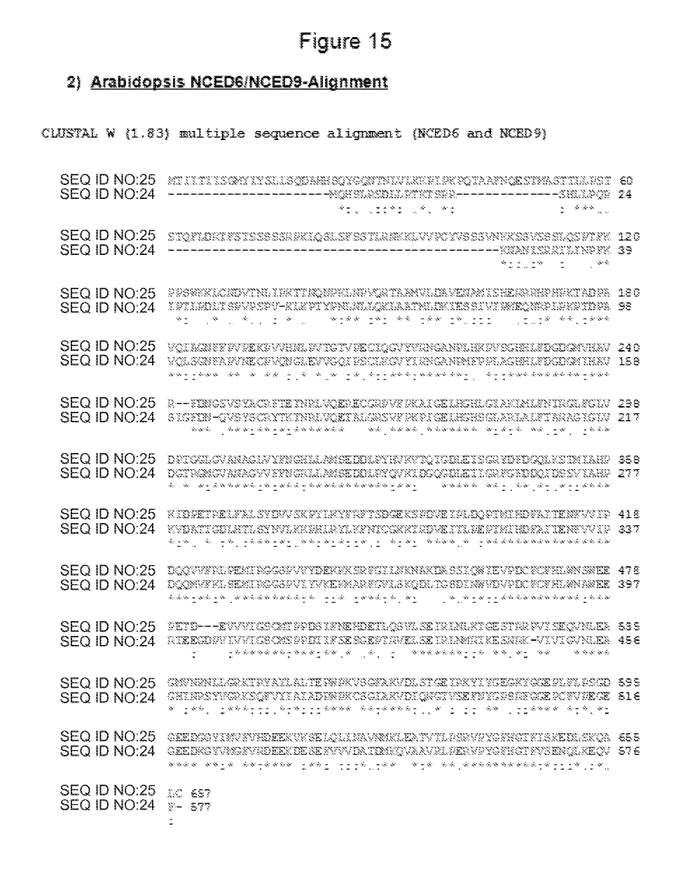

NCED genes from other species may be readily identified by standard homology searching of publicly available databases. For example, in one embodiment, seed specific NCED enzymes may be identified based on sequence homology and/or conserved residues selected from those depicted in FIG. 15.

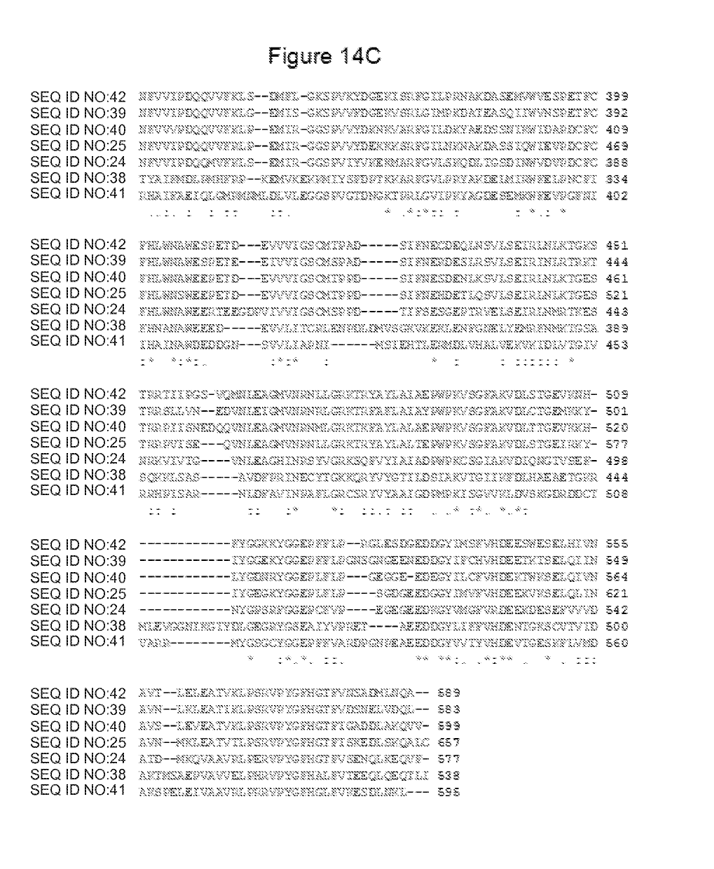

In one embodiment, the NCED comprises subsequences with sequence homology and/or conserved residues selected from those depicted in FIG. 14. In one embodiment, the NCED comprises an RPE65 domain and a PLN02258 domain.

In one embodiment, such seed-specific NCED can be identified based on the presence of one or more consensus sequences selected from DPAVQ (SEQ. ID. NO. 8), TNRLVQE (SEQ. ID. NO. 9), and VPDCFCFHLWN (SEQ. ID. NO. 10), wherein the sequence is present in the region(s) corresponding to that shown in FIG. 15. In one embodiment, seed-specific NCED genes may be identified based on the presence of one or more (e.g. all) consensus sequences selected from LLP (SEQ. ID. NO. 11), FDN (SEQ. ID. NO. 12), VSY (SEQ. ID. NO. 13), and DEEK (SEQ. ID. NO. 14), wherein the sequence(s) is/are present in the region(s) corresponding to that shown in FIG. 15.

In one embodiment, the seed-specific NCED comprises one or more (e.g. all) consensus sequences selected from DPAVQ (SEQ. ID. NO. 8), TNRLVQE (SEQ. ID. NO. 9), VPDCFCFHLWN (SEQ. ID. NO. 10), LLP (SEQ. ID. NO. 11), FDN (SEQ. ID. NO. 12), VSY (SEQ. ID. NO. 13), and DEEK (SEQ. ID. NO. 14), wherein the sequence(s) is/are present in the region(s) corresponding to that shown in FIG. 15.

For example, the Arabidopsis NCED6 depicted in FIG. 15 comprises DPAVQ (residues 96-100) (SEQ. ID. NO. 8), TNRLVQE (residues 175-181) (SEQ. ID. NO. 9), and VPDCFCFHLWN (residues 383-393) (SEQ. ID. NO. 10) while the Arabidopsis NCED9 depicted in FIG. 15 comprises DPAVQ (residues 178-182) (SEQ. ID. NO. 8), TNRLVQE (residues 256-262) (SEQ. ID. NO. 9), VPDCFCFHLWN (residues 464-474) (SEQ. ID. NO. 10), LLP (residues 56-58) (SEQ. ID. NO. 11), FDN (residues 242-244) (SEQ. ID. NO. 12), VSY (residues 247-249) (SEQ. ID. NO. 13), and DEEK (residues 609-612) (SEQ. ID. NO. 14).

In one embodiment, the seed-specific NCED comprises one or more sequences that are derivatives of the consensus sequences set forth above, for example, X₁X₂AVQ (SEQ. ID. NO. 15), X₃NX₄LVQE (SEQ. ID. NO. 16), and/or VPDCX₅X₆X₇X₈X₉X₁₀X₁₁ (SEQ. ID. NO. 17) in the region(s) corresponding to that shown in FIG. 15. With the teachings provided herein, the skilled artisan can readily select residues for any of X₁-X₁₁ to provide a functional NCED. For example, X₁-X₁₁ can be the corresponding residues depicted in FIG. 14, FIG. 15, or conservative substitutions thereof.

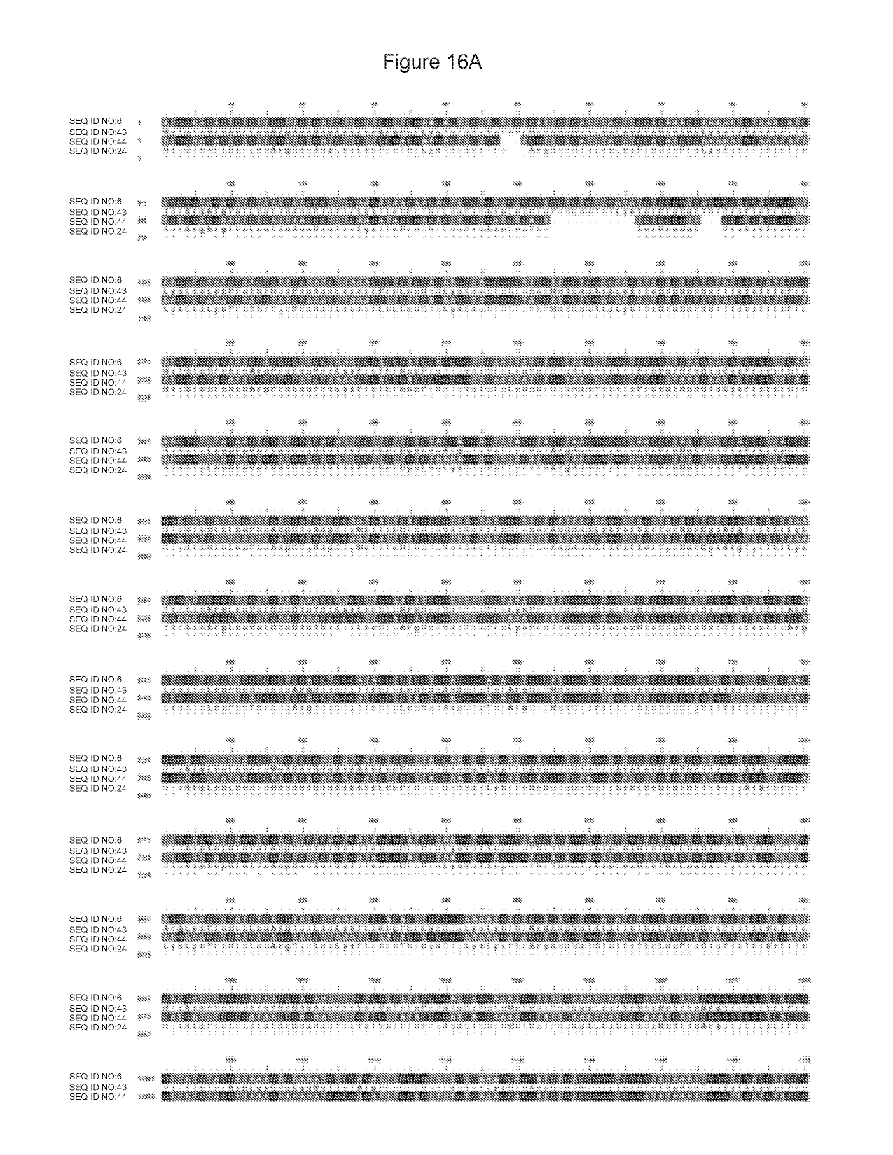

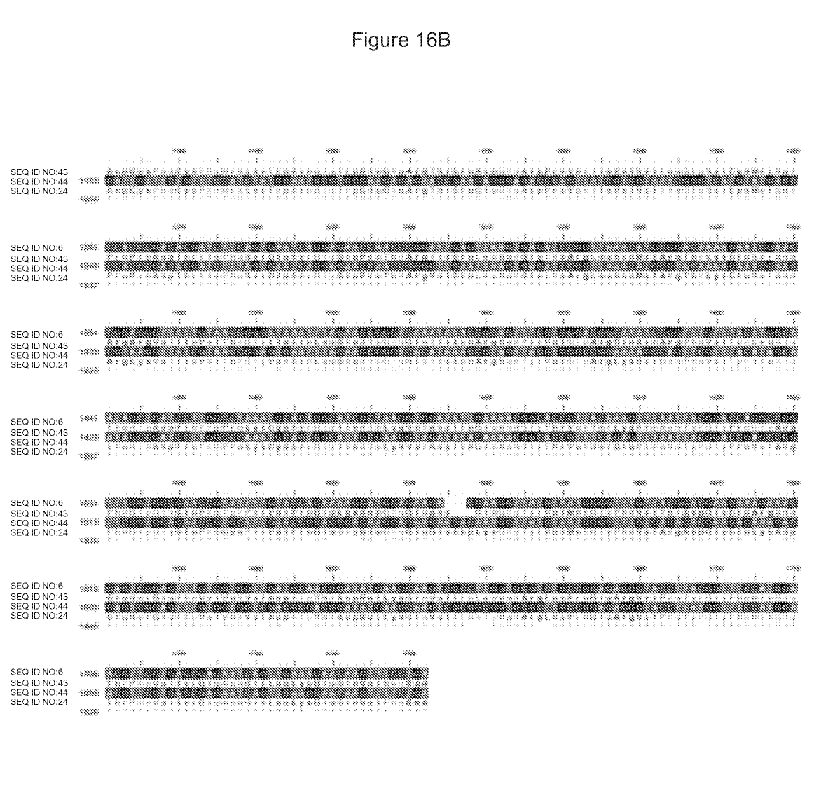

In one embodiment, the NCED is an NCED6. Optionally, the NCED6 contains subsequences with sequence homology and/or conserved residues selected from those depicted in FIG. 16. Useful NCED6 NCEDs can optionally contain one or more substitutions, insertions, and/or deletions, for example, relative to wild type NCED6 enzymes as depicted in FIG. 16.

Optionally, the NCED6 contains one or more (or all) consensus sequences selected from NAN (SEQ. ID. NO. 18), PTY (SEQ. ID. NO. 19), QNG (SEQ. ID. NO. 20), DGQ (SEQ. ID. NO. 21), DLTG (SEQ. ID. NO. 22), and SEF (SEQ. ID. NO. 23), wherein the sequence(s) is/are present in the region(s) corresponding to that shown in FIG. 15. For example, Arabidopsis NCED6 comprises the consensus sequence sequences in the locations set forth in Table D3.

TABLE D3
Arabidopsis NCED6 Subsequences

| Start | | End | |
|---|---|---|---|
| position | sequence | position | SEQ. ID. No. |
| 26 | NAN | 28 | (SEQ. ID. NO. 18) |
| 58 | PTY | 60 | (SEQ. ID. NO. 19) |
| 114 | QNG | 116 | (SEQ. ID. NO. 20) |
| 252 | DGQ | 254 | (SEQ. ID. NO. 21) |
| 372 | DLTG | 375 | (SEQ. ID. NO. 22) |
| 496 | SEF | 498 | (SEQ. ID. NO. 23) |

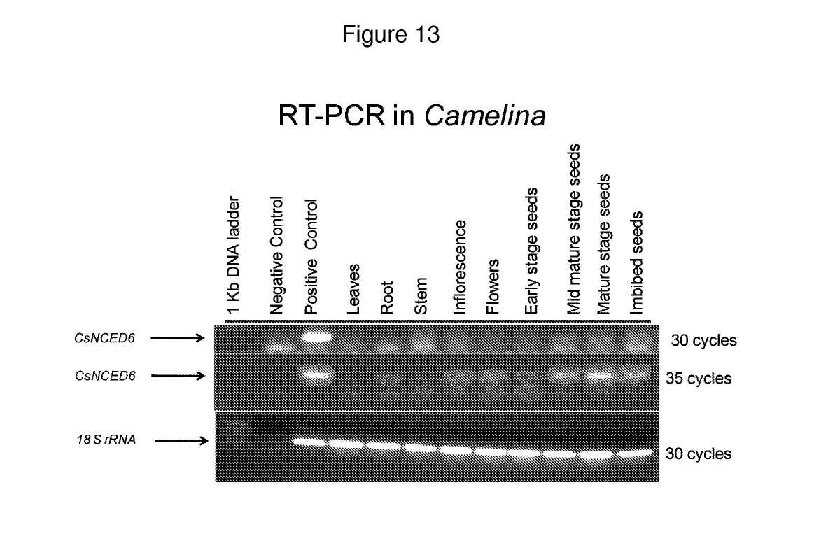

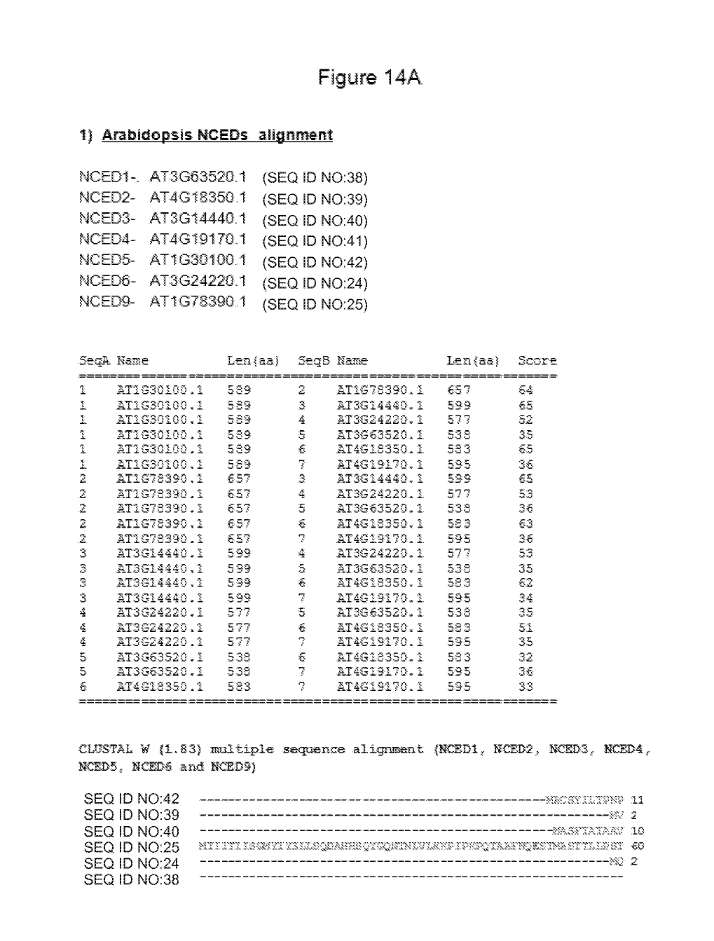

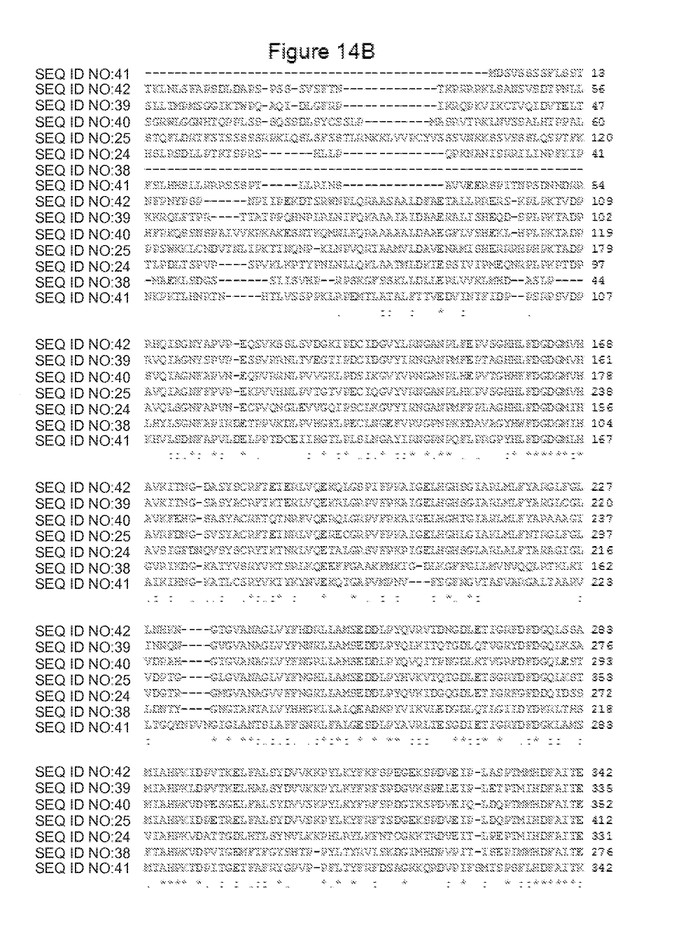

In one embodiment, the NCED is a seed-specific NCED. Surprisingly, the use of seed-specific NCEDs optionally provides superior control of seed germination, as taught herein. Useful seed-specific NCEDs include NCEDs which are primarily expressed in wild-type seeds such as mature or imbibed seeds (e.g. as depicted in FIG. 13) and NCEDs which are the primary (most abundant) NCED expressed in wild type seeds such as mature or imbibed seeds. For example, the seed specific NCED is an NCED6 or NCED9 such as an NCED6 or NCED9 from Arabidopsis or Camelina.

Although certain useful seed-specific NCEDs are expressed primarily in wild-type seeds, useful seed-specific NCEDs are not limited to such. For example, in one embodiment, the NCED is an NCED that is structurally related to the NCED6 and NCED9 enzymes from Arabidopsis or Camelina.

It is well established that the genetic code is degenerate and that some amino acids have multiple codons, and accordingly, multiple polynucleotides can encode the 9-cis-epoxycarotenoid dioxygenase of the invention. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode 9-cis-epoxycarotenoid dioxygenase proteins can be obtained from by “back-translation” (for example by using Computer programs such as “BackTranslate” (GCG™ Package, Acclerys, Inc. San Diego, Calif.) of the deduced coding sequences derived from 9-cis-epoxycarotenoid dioxygenase genomic clones, from cDNA or EST sequences, or any of the sequences listed in Table D2. Examples of nucleic acids that contain mature 9-cis-epoxycarotenoid dioxygenase protein-encoding nucleotide sequences for NCED6 include but are not limited to a sequence with at least 70%, at least 80%, at least 85%, at least 90%, at least 95%, at least 97%, at least 99%, or 100% sequence identity to SEQ. ID. No. 1 or SEQ. ID. No. 6.

Examples of nucleic acids that contain mature 9-cis-epoxycarotenoid dioxygenase protein-encoding nucleotide sequences for NCED9 include but are not limited to a sequence with at least 70%, at least 80%, at least 85%, at least 90%, at least 95%, at least 97%, at least 99%, or 100% sequence identity to SEQ. ID. No. 2.

In some embodiments, the non-native 9-cis-epoxycarotenoid dioxygenase-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the non-native 9-cis-epoxycarotenoid dioxygenase-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the non-native 9-cis-epoxycarotenoid dioxygenase-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the 9-cis-epoxycarotenoid dioxygenase polynucleotide sequences listed in Table D2. Polynucleotide “variants” may contain one or more substitutions, additions, deletions and/or insertions in relation to a reference polynucleotide. Generally, variants of the 9-cis-epoxycarotenoid dioxygenase reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence (i.e. to any of SEQ. ID, No. 1, 2, or 6) as determined by sequence alignment programs described elsewhere herein using default parameters.

In some embodiments the 9-cis-epoxycarotenoid dioxygenase which may be used in any of the methods and plants of the invention may have amino acid sequences which are substantially homologous, or substantially similar to any of the native 9-cis-epoxycarotenoid dioxygenase amino acid sequences, for example, to any of the native 9-cis-epoxycarotenoid dioxygenase amino acid sequences encoded by the genes listed in Table D2.

For use in the present invention, the 9-cis-epoxycarotenoid dioxygenase may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of a 9-cis-epoxycarotenoid dioxygenase, are also specifically included in any of the methods of the invention including for example, pyroglutamyl, iso-aspartyl, proteolytic, phosphorylated, glycosylated, reduced, oxidatized, isomerized, and deaminated variants of the 9-cis-epoxycarotenoid dioxygenase. For example although wild type NCEDs may contain a plastid targeting sequence, an NCED may be utilized according to the present invention with or without a plastid targeting sequence.

Alternatively, the 9-cis-epoxycarotenoid dioxygenase may have an amino acid sequence having at least 30% preferably at least 40, 50, 60, 70, 75, 80, 85, 90, 95, 98, or 99% identity with an 9-cis-epoxycarotenoid dioxygenase encoded by a gene listed in Table D2. In some embodiments, the 9-cis-epoxycarotenoid dioxygenase for use in any of the methods and plants of the present invention is at least 80% identical to the mature NCED6 from Arabidopsis thaliana (SEQ. ID. NO. 24).

(SEQ. ID. NO. 24)
MQHSLRSDLLPTKTSPRSHLLPQPKNANISRRILINPFKIPTLPDLTS
PVPSPVKLKPTYPNLNLLQKLAATMLDKIESSIVIPMEQNRPLPKPTD
PAVQLSGNFAPVNECPVQNGLEVVGQIPSCLKGVYIRNGANPMFPPLA
GHHLFDGDGMIHAVSIGFDNQVSYSCRYTKTNRLVQETALGRSVFPKP
IGELHGHSGLARLALFTARAGIGLVDGTRGMGVANAGVVFFNGRLLAM
SEDDLPYQVKIDGQGDLETIGRFGFDDQIDSSVIAHPKVDATTGDLHT
LSYNVLKKPHLRYLKFNTCGKKTRDVEITLPEPTMIHDFAITENFVVI
PDQQMVFKLSEMIRGGSPVIYVKEKMARFGVLSKQDLTGSDINWVDVP
DCFCFHLWNAWEERTEEGDPVIVVIGSCMSPPDTIFSESGEPTRVELS
EIRLNMRTKESNRKVIVTGVNLEAGHINRSYVGRKSQFVYIAIADPWP
KCSGIAKVDIQNGTVSEFNYGPSRFGGEPCFVPEGEGEEDKGYVMGFV
RDEEKDESEFVVVDATDMKQVAAVRLPERVPYGFHGTFVSENQLKEQV
F.

In some embodiments, the 9-cis-epoxycarotenoid dioxygenase for use in any of the methods and plants of the present invention is at least 80% identical to the mature NCED9 from Arabidopsis thaliana (SEQ. ID. NO. 25).

(SEQ. ID. NO. 25)
MTIITIISGMYIYSLLSQDAHHSQYGQNTNLVLKKPIPKPQTAAFNQE
STMASTTLLPSTSTQFLDRTFSTSSSSSRPKLQSLSFSSTLRNKKLVV
PCYVSSSVNKKSSVSSSLQSPTFKPPSWKKLCNDVTNLIPKTTNQNPK
LNPVQRTAAMVLDAVENAMISHERRRHPHPKTADPAVQIAGNFFPVPE
KPVVHNLPVTGTVPECIQGVYVRNGANPLHKPVSGHHLFDGDGMVHAV
RFDNGSVSYACRFTETNRLVQERECGRPVFPKAIGELHGHLGIAKLML
FNTRGLFGLVDPTGGLGVANAGLVYFNGHLLAMSEDDLPYHVKVTQTG
DLETSGRYDFDGQLKSTMIANPKIDPETRELFALSYDVVSKPYLKYFR
FTSDGEKSPDVEIPLDQPTMIHDFAITENFVVIPDQQVVFRLPEMIRG
GSPVVYDEKKKSRFGILNKNAKDASSIQWIEVPDCFCFHLWNSWEEPE
TDEVVVIGSCMTPPDSIFNEHDETLQSVLSEIRLNLKTGESTRRPVIS
EQVNLEAGMVNRNLLGRKTRYAYLALTEPWPKVSGFAKVDLSTGEIRK
YIYGEGKYGGEPLFLPSGDGEEDGGYIMVFVHDEEKVKSELQLINAVN
MKLEATVTLPSRVPYGFHGTFISKEDLSKQALC.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into 9-cis-epoxycarotenoid dioxygenase and are considered within the scope of the invention. Mutations of 9-cis-epoxycarotenoid dioxygenase that increase the activity of the protein are known and may be used in the Methods and plants of the invention. The 9-cis-epoxycarotenoid dioxygenase may thus include one or more amino acid deletions, additions, insertions, and/or substitutions based on any of the naturally-occurring isoforms of 9-cis-epoxycarotenoid dioxygenase. These may be contiguous or non-contiguous. Representative variants may include those having 1 to 8, or more preferably 1 to 4, 1 to 3, or 1 or 2 amino acid substitutions, insertions, and/or deletions as compared to any of sequences listed in Table D2.

The variants, derivatives, and fusion proteins of 9-cis-epoxycarotenoid dioxygenase are functionally equivalent in that they have detectable 9-cis-epoxycarotenoid dioxygenase activity. More particularly, they exhibit at least 5%, at least 10%, at least 20%, at least 30%, at least 40%, preferably at least 60%, more preferably at least 80% of the activity of 9-cis-epoxycarotenoid dioxygenase such as NCED6 from Arabidopsis thaliana, SEQ. ID. NO. 24, and are thus they are capable of substituting for 9-cis-epoxycarotenoid dioxygenase itself.

All such variants, derivatives, fusion proteins, or fragments of 9-cis-epoxycarotenoid dioxygenase are included, and may be used in any of the polynucleotides, vectors, host cell and Methods disclosed and/or claimed herein, and are subsumed under the term “9-cis-epoxycarotenoid dioxygenase”. Suitable assays for determining functional 9-cis-epoxycarotenoid dioxygenase activity are well known in the art.

### Exemplary Gibberellin (GA) Synthesis Genes

In one embodiment, the hormone-regulating gene encodes an enzyme which catalyzes a step in the synthesis of GA. Such a gene may be useful to release a seed from dormancy or induce germination. Optionally, the enzyme is a GA oxidase such as GA3 oxidase (e.g. GA3ox1 or GA3ox2). Other useful GA synthesis enzymes include GA3ox3, GA3ox4 and GA2ox1.

In any of these methods, DNA constructs, and transgenic organisms, the terms “Gibberellin oxidase” or “GA3 oxidase” refers to all naturally-occurring and synthetic genes encoding an GA3 oxidase capable of forming an gibberellin active in inducing seed germination.

In one aspect the GA3 oxidase is from planta. In a further embodiment the GA3 oxidase is from Arabidopsis thaliana. Representative species and Gene bank accession numbers for various species of GA3 oxidase are listed below in Table D4, and genes from other species may be readily identified by standard homology searching of publicly available databases.

TABLE D4
Exemplary GA3 oxidases

| Uniprot | | |
|---|---|---|
| Accession | Name | Organism |
| Q39103 | Gibberellin 3-beta-dioxygenase 1 | Arabidopsis thaliana (Mouse-ear cress) |
| Q0WTG6 | Gibberillin 3 beta-hydroxylase | Arabidopsis thaliana (Mouse-ear cress) |
| D7KDH1 | Putative uncharacterized | Arabidopsis lyrata subsp. lyrata |
| | protein | |
| D5L701 | GA3ox1-like protein | Camelina sativa (False flax) (Gold-of- |
| | | pleasure) |
| D7KX68 | Predicted protein | Arabidopsis lyrata subsp. lyrata |
| Q9ZT84 | Gibberellin 3-beta-dioxygenase 2 | Arabidopsis thaliana (Mouse-ear cress) |
| Q59J01 | Gibberellin 3-beta hydroxylase | Prunus subhirtella |
| B9GEI2 | Gibberellin 3-oxidase | Populus trichocarpa (Western balsam poplar) |
| | | (Populus balsamifera subsp. trichocarpa) |
| B9GWQ8 | Gibberellin 3-oxidase | Populus trichocarpa (Western balsam poplar) |
| | | (Populus balsamifera subsp. trichocarpa) |
| Q6T6I0 | Gibberellin 3-oxidase | Populus tremula × Populus tremuloides |
| D7F2B0 | Gibberellin 3-oxidase | Malus domestica (Apple) (Pyrus malus) |
| D7U0Q5 | Whole genome shotgun | Vitis vinifera (Grape) |
| | sequence of line PN40024 . . . | |
| A5AJH8 | Putative uncharacterized | Vitis vinifera (Grape) |
| | protein | |
| A9P5X2 | Gibberellin 3-beta-hydroxylase | Medicago falcata (Sickle medic) |
| A9P5X4 | Gibberellin 3-beta-hydroxylase | Medicago sativa subsp. caerulea |
| O24648 | 2-oxoglutarate-dependent | Pisum sativum (Garden pea) |
| | dioxygenase | |
| O24623 | Gibberellin 3 beta-hydroxylase | Pisum sativum (Garden pea) |
| A9P5X3 | Gibberellin 3-beta-hydroxylase | Medicago sativa subsp. caerulea |
| O24627 | Defective gibberellin 3B- | Pisum sativum (Garden pea) |
| | hydroxylase | |
| O22377 | Gibberellin 3 beta-hydroxylase | Pisum sativum (Garden pea) |
| Q9SLQ9 | Gibberellin 3beta-hydroxylase | Nicotiana tabacum (Common tobacco) |
| Q9ZWQ0 | Gibberelin 3beta-hydroxylase | Lactuca sativa (Garden lettuce) |
| B2ZZ95 | Gibberellin 3-oxidase2 | Chrysanthemum morifolium (Florist's daisy) |
| | | (Dendranthema grandiflorum) |
| Q0ZBL9 | Gibberellin 3-oxidase 1 | Rumex palustris |
| B5AK92 | Gibberellin 3-beta hydroxylase | Chrysanthemum morifolium (Florist's daisy) |
| | | (Dendranthema grandiflorum) |
| Q8S309 | Gibberellin 3-oxidase 2 | Nicotiana sylvestris (Wood tobacco) |
| B2ZZ94 | Gibberellin 3-oxidase1 | Chrysanthemum morifolium (Florist's daisy) |
| | | (Dendranthema grandiflorum) |
| Q1AE44 | Gibberellin 3-oxidase | Fragaria ananassa (Strawberry) |
| Q8GT57 | Gibberellin 3-oxidase | Cucurbita maxima (Pumpkin) (Winter |
| | | squash) |
| Q9ZWR7 | 3b-hydroxylase | Solanum lycopersicum (Tomato) |
| | | (Lycopersicon esculentum) |
| Q76I30 | Gibberellin 3-oxidase-like | Ipomoea nil (Japanese morning glory) |
| | protein | (Pharbitis nil) |
| Q8RVP0 | Gibberellin 3-oxidase 1 | Nicotiana sylvestris (Wood tobacco) |
| D7U0Q8 | Whole genome shotgun | Vitis vinifera (Grape) |
| | sequence of line PN40024 . . . | |
| A4URE7 | Gibberellin 3-oxidase 2 | Nicotiana tabacum (Common tobacco) |
| C0LZW9 | Gibberellin 3-oxidase | Solanum tuberosum subsp. andigena |
| Q941N1 | Gibberellin 3-beta-hydroxylase 1 | Solanum tuberosum (Potato) |
| C0LZW8 | Gibberellin 3-oxidase | Solanum tuberosum subsp. andigena |
| A5ASP9 | Putative uncharacterized | Vitis vinifera (Grape) |
| | protein | |
| Q9ZWP9 | Gibberellin 3beta-hydroxylase | Lactuca sativa (Garden lettuce) |
| D7KX69 | Gibberellin 3-oxidase 4 | Arabidopsis lyrata subsp. lyrata |
| Q9C971 | Gibberellin 3-beta-dioxygenase 4 | Arabidopsis thaliana (Mouse-ear cress) |
| Q9ZWR6 | 3b-hydroxylase | Solanum lycopersicum (Tomato) |
| | | (Lycopersicon esculentum) |
| Q941N2 | Gibberellin 3-beta-hydroxylase 2 | Solanum tuberosum (Potato) |
| Q8GSN7 | Gibberellin 3-oxidase | Spinacia oleracea (Spinach) |
| Q6F6H6 | Gibberellin 3beta-hydroxylase2 | Daucus carota (Carrot) |
| Q6F6H7 | Gibberellin 3beta-hydroxylase1 | Daucus carota (Carrot) |
| B2NI88 | Gibberellin 3-oxidase | Allium fistulosum (Welsh onion) |
| Q9M4P2 | Gibberellin 3-beta-hydroxylase | Cucurbita maxima (Pumpkin) (Winter |
| | | squash) |
| B9IKS1 | Gibberellin 3-oxidase | Populus trichocarpa (Western balsam poplar) |
| | | (Populus balsamifera subsp. trichocarpa) |
| D7SUP3 | Whole genome shotgun | Vitis vinifera (Grape) |
| | sequence of line PN40024 . . . | |
| Q84XY3 | GA4 | Brassica rapa subsp. pekinensis (Chinese |
| | | cabbage) |
| B9RP90 | Gibberellin 3-beta hydroxylase, | Ricinus communis (Castor bean) |
| | putative | |
| B9RK13 | Gibberellin 3-beta hydroxylase, | Ricinus communis (Castor bean) |
| | putative | |
| B2BA73 | Gibberellin 3-oxidase | Pisum sativum (Garden pea) |
| Q6F6H5 | Gibberellin 3beta-hydroxylase3 | Daucus carota (Carrot) |
| B9RPD0 | Gibberellin 3-beta hydroxylase, | Ricinus communis (Castor bean) |
| | putative | |
| Q76I29 | Gibberellin 3-oxidase-like | Ipomoea nil (Japanese morning glory) |
| | protein | (Pharbitis nil) |
| Q9FXW0 | Gibberellin 3b-hydroxylase No3 | Lactuca sativa (Garden lettuce) |
| C7TQK2 | Putative GA3OX protein | Rosa luciae |
| Q9SVS8 | Gibberellin 3-beta-dioxygenase 3 | Arabidopsis thaliana (Mouse-ear cress) |
| A0MF88 | Putative uncharacterized | Arabidopsis thaliana (Mouse-ear cress) |
| | protein | |
| D7MEQ4 | Gibberellin 3-oxidase 3 | Arabidopsis lyrata subsp. lyrata |
| Q4A3R2 | Gibberellin 3-oxidase | Phaseolus coccineus (Scarlet runner bean) |
| | | (Phaseolus multiflorus) |
| Q9LM06 | Putative gibberellin 3 beta | Citrullus lanatus (Watermelon) |
| | hydroxylase | (Citrullus vulgaris) |
| C5XMS7 | Putative uncharacterized | Sorghum bicolor (Sorghum) |
| | protein Sb03g004020 | (Sorghum vulgare) |
| B9RUX0 | Gibberellin 3-beta hydroxylase, | Ricinus communis (Castor bean) |
| | putative | |
| Q9FU53 | GA 3beta-hydroxylase | Oryza sativa subsp. japonica (Rice) |
| Q94IE4 | GA 3beta-hydroxylase | Oryza sativa (Rice) |
| A2WLB3 | Putative uncharacterized | Oryza sativa subsp. indica (Rice) |
| | protein | |
| Q94ID4 | GA 3beta-hydroxylase | Oryza sativa (Rice) |
| Q0JQ78 | Os01g0177400 protein | Oryza sativa subsp. japonica (Rice) |
| Q3I409 | Gibberellin 3-beta-dioxygenase | Triticum aestivum (Wheat) |
| | 2-3 | |
| Q673G4 | GA 3-oxidase 2 | Hordeum vulgare var. distichum (Two- |
| | | rowed barley) |
| Q60FR6 | Gibberellin 3beta-hydroxylase | Hordeum vulgare (Barley) |
| Q673G5 | GA 3-oxidase 1 | Hordeum vulgare var. distichum (Two- |
| | | rowed barley) |
| Q3I411 | Gibberellin 3-beta-dioxygenase | Triticum aestivum (Wheat) |
| | 2-1 | |
| Q3I410 | Gibberellin 3-beta-dioxygenase | Triticum aestivum (Wheat) |
| | 2-2 | |
| D7MYG0 | Putative uncharacterized | Arabidopsis lyrata subsp. lyrata |
| | protein | |
| O24417 | Gibberellin 2beta,3beta- | Cucurbita maxima (Pumpkin) (Winter |
| | hydroxylase | squash) |
| A8D8H3 | Gibberellin 3-beta-hydroxylase | Dasypyrum villosum |
| A9LY27 | Gibberellin 3-oxidase-like | Selaginella moellendorffii |
| | protein | |
| B3V744 | Gibberellin 3 beta oxidase | Sisymbrium officinale |
| D5L0B2 | Putative 2-oxoglutarate | Avena sativa (Oat) |
| | dependent dioxygenase | |
| Q94IE3 | GA 3beta-hydroxylase | Oryza sativa (Rice) |
| Q6AT12 | Os05g0178100 protein | Oryza sativa subsp. japonica (Rice) |
| B8AYM7 | Putative uncharacterized | Oryza sativa subsp. indica (Rice) |
| | protein | |
| Q9AYQ9 | Gibberellin-3-beta-hydroxylase | Eustoma grandiflorum (Bluebells) |
| | | (Lisianthus russellianus) |
| D0PQ20 | GA3-oxidase | Rhynchoryza subulata |
| D0PQ19 | GA3-oxidase | Chikusichloa aquatica |
| D0PQ21 | GA3-oxidase | Ehrharta erecta |
| D0PQ17 | GA3-oxidase | Oryza granulata |
| D0PQ13 | GA3-oxidase | Oryza punctata (Red rice) |
| D0PQ14 | GA3-oxidase | Oryza officinalis |
| D0PQ15 | GA3-oxidase | Oryza australiensis |
| D0PQ12 | GA3-oxidase | Oryza meridionalis |
| D0PQ16 | GA3-oxidase | Oryza brachyantha |
| B4FQ42 | Putative uncharacterized | Zea mays (Maize) |
| | protein | |
| B8A213 | Putative uncharacterized | Zea mays (Maize) |
| | protein | |
| D0PQ18 | GA3-oxidase | Luziola fluitans |
| B6UAD7 | Gibberellin 3-beta-dioxygenase | Zea mays (Maize) |
| | 2-2 | |
| B8A259 | Putative uncharacterized | Zea mays (Maize) |
| | protein | |
| C5Z166 | Putative uncharacterized | Sorghum bicolor (Sorghum) |
| | protein Sb09g005400 | (Sorghum vulgare) |
| D7L9V2 | Oxidoreductase | Arabidopsis lyrata subsp. lyrata |
| Q9SRM3 | Leucoanthocyanidin | Arabidopsis thaliana (Mouse-ear cress) |
| | dioxygenase, putative; 414 . . . | |
| Q7FAL4 | OSJNBa0064M23.14 protein | Oryza sativa subsp. japonica (Rice) |
| B9FC47 | Putative uncharacterized | Oryza sativa subsp. japonica (Rice) |
| | protein | |
| A9NQA3 | Putative uncharacterized | Picea sitchensis (Sitka spruce) |
| | protein | |
| B9T5W6 | Leucoanthocyanidin | Ricinus communis (Castor bean) |
| | dioxygenase, putative | |
| Q9FFF6 | AT5g05600/MOP10_14 | Arabidopsis thaliana (Mouse-ear cress) |
| Q8LF06 | Leucoanthocyanidin | Arabidopsis thaliana (Mouse-ear cress) |
| | dioxygenase-like protein | |
| Q5QLC8 | Os01g0832600 protein | Oryza sativa subsp. japonica (Rice) |
| D7LYY9 | Oxidoreductase | Arabidopsis lyrata subsp. lyrata |
| B8ABN6 | Putative uncharacterized | Oryza sativa subsp. indica (Rice) |
| | protein | |
| Q9ZWQ9 | Flavonol synthase/flavanone | Citrus unshiu (Satsuma mandarin) |
| | 3-hydroxylase | (Citrus nobilis var. unshiu) |
| A8QKF0 | Flavonol synthase | Rudbeckia hirta (Black-eyed Susan) |
| B9RT28 | Flavonol synthase/flavanone | Ricinus communis (Castor bean) |
| | 3-hydroxylase, pu . . . | |
| Q0H3G8 | Flavanone 3-hydroxylase | Triticum aestivum (Wheat) |
| D7TJX3 | Whole genome shotgun | Vitis vinifera (Grape) |
| | sequence of line PN40024 . . . | |
| D7SM30 | Whole genome shotgun | Vitis vinifera (Grape) |
| | sequence of line PN40024 . . . | |
| B9S1A0 | Flavonol synthase/flavanone | Ricinus communis (Castor bean) |
| | 3-hydroxylase, pu . . . | |
| A5AZG3 | Putative uncharacterized | Vitis vinifera (Grape) |
| | protein | |
| D7TPQ0 | Whole genome shotgun | Vitis vinifera (Grape) |
| | sequence of line PN40024 . . . | |
| B9S192 | Flavonol synthase/flavanone | Ricinus communis (Castor bean) |
| | 3-hydroxylase, pu . . . | |
| B9HSN0 | Predicted protein | Populus trichocarpa (Western balsam poplar) |
| | | (Populus balsamifera subsp. trichocarpa) |
| A9LY24 | Gibberellin 3-oxidase-like | Physcomitrella patens (Moss) |
| | protein | |
| D7MY61 | Oxidoreductase | Arabidopsis lyrata subsp. lyrata |
| Q8H0F3 | GA 3bete-hydroxylase | Cucumis sativus (Cucumber) |

It is well established that the genetic code is degenerate and that some amino acids have multiple codons, and accordingly, multiple polynucleotides can encode the GA3 oxidase of the invention. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode GA3 oxidase proteins can be obtained from by “back-translation” (for example by using Computer programs such as “BackTranslate” (GCG™ Package, Acclerys, Inc. San Diego, Calif.) of the deduced coding sequences derived from GA3 oxidase genomic clones, from cDNA or EST sequences, or any of the sequences listed in Table D4. Examples of nucleic acids that contain mature GA3 oxidase protein-encoding nucleotide sequences include but are not limited to a sequence with at least 70%, at least 80%, at least 85%, at least 90%, at least 95%, at least 97%, at least 99%, or 100% sequence identity to SEQ. ID. No. 4.

In some embodiments, the non-native GA3 oxidase-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the non-native GA3 oxidase-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the non-native GA3 oxidase-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript.

A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the GA3 oxidase polynucleotide sequences listed in Table D4. Polynucleotide “variants” may contain one or more substitutions, additions, deletions and/or insertions in relation to a reference polynucleotide. Generally, variants of the GA3 oxidase reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence (i.e. to SEQ. ID, No. 4) as determined by sequence alignment programs described elsewhere herein using default parameters.

In some embodiments the GA3 oxidase which may be used in any of the methods and plants of the invention may have amino acid sequences which are substantially homologous, or substantially similar to any of the native GA3 oxidase amino acid sequences, for example, to any of the native GA3 oxidase amino acid sequences encoded by the genes listed in Table D4.

For use in the present invention, the GA3 oxidase may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of a GA3 oxidase, are also specifically included in any of the methods of the invention including for example, pyroglutamyl, iso-aspartyl, proteolytic, phosphorylated, glycosylated, reduced, oxidatized, isomerized, and deaminated variants of the GA3 oxidase.

Alternatively, the GA3 oxidase may have an amino acid sequence having at least 30% preferably at least 40, 50, 60, 70, 75, 80, 85, 90, 95, 98, or 99% identity with an GA3 oxidase encoded by a gene listed in Table D4. In a preferred embodiment, the GA3 oxidase for use in any of the methods and plants of the present invention is at least 80% identical to the mature GA3 oxidase from Arabidopsis thaliana (SEQ. ID. NO. 26).

(SEQ. ID. NO. 26)
MPAMLTDVFRGHPIHLPHSHIPDFTSLRELPDSYKWTPKDDLLFSAAP
SPPATGENIPLIDLDHPDATNQIGHACRTWGAFQISNHGVPLGLLQDI
EFLTGSLFGLPVQRKLKSARSETGVSGYGVARIASFFNKQMWSEGFTI
TGSPLNDFRKLWPQHHLNYCDIVEEYEEHMKKLASKLMWLALNSLGVS
EEDIEWASLSSDLNWAQAALQLNHYPVCPEPDRAMGLAAHTDSTLLTI
LYQNNTAGLQVFRDDLGWVTVPPFPGSLVVNVGDLFHILSNGLFKSVL
HRARVNQTRARLSVAFLWGPQSDIKISPVPKLVSPVESPLYQSVTWKE
YLRTKATHFNKALSMIRNHREE.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into GA3 oxidase and are considered within the scope of the invention. Mutations of GA3 oxidase that increase the activity of the protein are known and may be used in the methods and plants of the invention. The GA3 oxidase may thus include one or more amino acid deletions, additions, insertions, and/or substitutions based on any of the naturally-occurring isoforms of GA3 oxidase. These may be contiguous or non-contiguous. Representative variants may include those having 1 to 8, or more preferably 1 to 4, 1 to 3, or 1 or 2 amino acid substitutions, insertions, and/or deletions as compared to any of sequences listed in Table D4.

The variants, derivatives, and fusion proteins of GA3 oxidase are functionally equivalent in that they have detectable GA3 oxidase activity. More particularly, they exhibit at least 5%, at least 10%, at least 20%, at least 30%, at least 40%, preferably at least 60%, more preferably at least 80% of the activity of GA3 oxidase from Arabidopsis thaliana, SEQ. ID. NO. 26, and are thus they are capable of substituting for GA3 oxidase itself.

All such variants, derivatives, fusion proteins, or fragments of GA3 oxidase are included, and may be used in any of the polynucleotides, vectors, host cell and methods disclosed and/or claimed herein, and are subsumed under the term “GA3 oxidase”. Suitable assays for determining functional GA3 oxidase activity are well known in the art.

In one embodiment, the hormone-regulating gene encodes an enzyme which catalyzes a step in the deactivation of GA. Such a gene may be useful to maintain dormancy or prevent germination. Examples of GA deactivation enzymes include GA2 oxidases and GA methyltransferases.

Combinations of Hormone-Regulating Genes

In one embodiment, a genetically modified plant comprises a combination of hormone-regulating genes.

In one embodiment, a plant is transformed with both a seed dormancy gene and a seed germination gene, for example, as set forth in Table D5. One or both of the seed dormancy gene and a seed germination gene can be operably linked to an inducible promoter (e.g. of a gene switch). The seed-dormancy gene can be expressed to induce seed dormancy and the seed germination gene can be expressed to rescue the seed from dormancy, i.e. to induce seed germination.

TABLE D5
Gene Combinations

| Seed Dormancy Gene | Seed Germination Gene |
|---|---|
| NCED (e.g. NCED6) | NCED (e.g. NCED6) RNAi agent |
| NCED (e.g. NCED6) | ABA 8′ Hydroxylase |
| NCED (e.g. NCED6) | GA3 oxidase (e.g. GA3ox1) |
| ABA 8′ Hydroxylase RNAi | ABA 8′ Hydroxylase (over expression) |
| ABA 8′ Hydroxylase RNAi | ABA 8′ Hydroxylase and GA3 oxidase |
| ABA 8′ Hydroxylase RNAi | GA3 oxidase |
| GA3 oxidase RNAi | GA3 oxidase (over expression) |

While either or both of the seed dormancy gene and the seed germination gene can be operably linked to an inducible promoter, the host can be transformed with the seed dormancy gene and the seed germination gene in any suitable manner. For example, the seed dormancy and the seed germination gene can be expressed under the control of a different gene switches. In another example, the seed dormancy gene can be operably linked to a seed-specific promoter and the seed germination gene can be expressed under the control of a gene switch (which can also include a seed-specific promoter).

As set forth in Table D5 an RNAi construct which targets a seed germination gene (e.g. ABA 8′ hydroxylase RNAi) can be provided as the seed dormancy gene (e.g. linked to a seed-specific promoter or the native promoter of the target gene). However, in an alternative embodiment, a gene knockout such as a null mutation can be imparted to the plant instead of the RNAi construct. Seed germination can then be made inducible by transforming the plant with a seed germination gene (e.g. ABA 8′ Hydroxylase) under the control of a gene switch.

Exemplary Plant Gene Switch Systems (PGSS)

According to the present invention, a hormone-regulating gene may be placed under the control of a gene switch which comprises a receptor fusion protein encoding a trans-acting factor that responds to a suitable ligand to activate the gene expression of the hormone regulating gene via an interaction with the cis acting elements in the expression cassette to which the hormone-regulating gene is operatively coupled.

The activity of the trans-acting factor, and thus, transcription of the hormone-regulating gene, is regulated by the presence or absence of a chemical modulator of the trans-acting factor. Surprisingly, seed germination may now be controlled at will by administering or withholding the chemical modulator.

The expression control sequences for the hormone regulating gene comprises a cis-element such as UAS elements capable of binding the trans-acting factor. The activity of the trans-acting factor is governed by the presence or absence of the chemical modulator, for example, by a conformational change induced by binding of the chemical modulator to the trans-acting factor. Typically, regulation of transcription occurs when the trans-acting factor binds the cis-element in an active conformation.

Any gene switch is useful according to the present invention. For example, gene switches modulated by ecdysone modulators, tetracyclines, steroids, glucocorticoids, estradiols, salicylic acid, and ethanol are known in the art. (See generally, Venkata et al., Chapter 21 Ecdysone: Structures and Functions Springer+Business Media B.V. 2009)

Exemplary Trans-Acting Factors

A trans-acting factor of the present invention can be any trans-acting factor that, when bound by a chemical modulator, binds a cis-element of a promoter and regulates the transcriptional activity of that promoter.

The trans-acting factor may be a transcription inducer (e.g. transcription factor) or a transcription blocker (e.g. transcription repressor). A trans-acting factor is a transcription inducer when binding of the trans-acting factor (in an active formation) to the cis-element initiates transcription of an operably linked polynucleotide. A trans-acting factor is a transcription blocker when binding of the trans-acting factor in an active formation to the cis-element blocks transcription of an operably linked polynucleotide. Accordingly, transcription of a polynucleotide is regulated (e.g. up-regulated or down-regulated) by the presence or absence of one or more chemical modulators that interact with (e.g. bind) the trans-acting factor.

A plant of the present invention comprises a genetic construct comprising an expressible gene encoding a trans-acting factor. The trans-acting factor and/or the genetic construct may be exogenous or endogenous to the host. An exogenous trans-acting factor may be expressed in a host plant by transforming the plant with an expressible gene encoding the trans-acting factor.

Useful trans-acting factors include those which are modulated by ecdysone modulators, tetracyclines, steroids, (such as FXR, RXR, glucocorticoids, and estradiols), salicylic acid, or ethanol.

In one embodiment, the trans-acting factor comprises an ecdysone receptor (EcR). Exemplary EcRs have one or more of the following features:

a. an N-terminal A/B domain;

b. a DNA-binding C domain;

c. a hinge (D) region;

d. a ligand-binding E domain; a

e. C-terminal F domain;

f. bind an ecdysone-response [cis-]element (EcRE);

g. regulate transcription in the presence of an ecdysone modulator;

h. heterodimerize with nuclear receptors such as ultraspiracle protein.

A number of useful EcRs are known in the art, and have been used to develop ligand regulated gene switches. Specific examples of EcR based gene switches include for example those disclosed in U.S. Pat. No. 6,723,531, U.S. Pat. No. 5,514,578, U.S. Pat. No. 6,245,531, U.S. Pat. No. 6,504,082, U.S. Pat. No. 7,151,168, U.S. Pat. No. 7,205,455, U.S. Pat. No. 7,238,859, U.S. Pat. No. 7,456,315, U.S. Pat. No. 7,563,928, U.S. Pat. No. 7,091,038, U.S. Pat. No. 7,531,326, U.S. Pat. No. 7,776,587, U.S. Pat. No. 7,807,417, U.S. Pat. No. 7,601,508, U.S. Pat. No. 7,829,676, U.S. Pat. No. 7,919,269, U.S. Pat. No. 7,563,879, U.S. Pat. No. 7,297,781, U.S. Pat. No. 7,312,322, U.S. Pat. No. 6,379,945,U.S. Pat. No. 6,610,828, U.S. Pat. No. 7,183,061 and U.S. Pat. No. 7,935,510.

For example, naturally occurring EcRs include the ligand-controlled transcription factors found in insects as members of the nuclear and steroidal receptor families that regulate growth, molting, and development in insects by controlling the activity of ecdysteroids. The EcR heterodimerizes with other members of the nuclear receptor family such as ultraspiracle protein (USP). The EcR/USP heterodimer binds to the ecdysone response elements (EcREs) present in the promoter regions of ecdysone response genes and regulate their transcription in ligand-controlled manner.

A useful trans-acting factor receptor can be produced as a receptor fusion protein (e.g. chimeric receptor protein) that responds to a suitable ligand to activate gene expression. A useful receptor fusion protein can comprise:

a. an activation domain

b. a DNA binding domain; and

c. a ligand-binding region.

Exemplary activation domains include for example the VP16 activation domain from herpes simplex virus, and the rice bZIP protein RF2a, as provided in Table D6.

TABLE D6.

| Name | Sequence | SEQ ID. NO. |
|---|---|---|
| VP16 amino | KVAPPTDVSL GDELHLDGED | SEQ. ID. |
| acids 413-490 | VAMAHADALD DFDLDMLGDG | No. 27 |
| | DSPGPGFTPH DSAPYGALDM | |
| | ADFEFEQMFT DALGIDEYGG | |
| | EFPGIRR | |
| Rice RF2 | MNREKSPIPG DGGDGLPPQA | SEQ. ID. |
| | TRRAGPPAAA AAAEYDISRM | No. 28 |
| | PDFPTRNPGH RRAHSEILSL | |
| | PEDLDLCAAG GGDGPSLSDE | |
| | NDEELFSMFL DVEKLNSTCG | |
| | ASSEAEAESS SAAAHGARPK | |
| | HQHSLSMDES MSIKAEELVG | |
| | ASPGTEGMSS AEAKKAVSAV | |
| | KLAELALVDP KRAKRIWANR | |
| | QSAARSKERK MRYIAELERK | |
| | VQTLQTEATT LSAQLALLQR | |
| | DTSGLTTENS ELKLRLQTME | |
| | QQVHLQDALN DTLKSEVQRL | |
| | KVATGQMANG GGMMMNFGGM | |
| | PHQFGGNQQM FQNNQAMQSM | |
| | LAAHQLQQLQ LHPQAQQQQV | |
| | LHPQHQQQQP LHPLQAQQLQ | |
| | QAARDLKMKS PMGGQSQWGD | |
| | GKSGSSGN | |

The activation domain can be, for example, a VP16 activation domain from herpes simplex virus (e.g. V, amino acids 413-490), as seen in a VGE vector, or an activation domain from rice bZIP protein RF2a (for example amino acids 49 to 116, or 56 to 84 of SEQ. ID. No. 28) comprising the active subdomain of RF2a (A) (23), as seen in an AGE vector. Similar activation domains from other species may be readily identified by standard homology searching of publicly available databases (See for example, Ordiz et al., (2010) Plant Biotech. J. 8 835-844).

It is well established that the genetic code is degenerate and that some amino acids have multiple codons, and accordingly, multiple polynucleotides can encode the activation domain used in the trans acting factor. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode the activation domain can be obtained from by “back-translation” (for example by using Computer programs such as “BackTranslate” (GCG™ Package, Acclerys, Inc. San Diego, Calif.) of the deduced coding sequences derived from the activation domain of transcription factors identified from genomic clones, from cDNA or EST sequences, or any of the sequences listed in Table D6.

In some embodiments, the activation domain-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the activation domain-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the activation domain-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the activation domain sequences listed in Table D6. Polynucleotide “variants” may contain one or more substitutions, additions, deletions and/or insertions in relation to a reference polynucleotide. Generally, variants of the activation domain reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence as determined by sequence alignment programs described elsewhere herein using default parameters.

In some embodiments the activation domain which may be used in any of the methods and plants of the invention may have amino acid sequences which are substantially homologous, or substantially similar to any of the native activation domain amino acid sequences listed in Table D6.

For use in the present invention, the activation domain may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of activation domain, are also specifically included in any of the methods of the invention including for example, pyroglutamyl, iso-aspartyl, proteolytic, phosphorylated, glycosylated, reduced, oxidatized, isomerized, and deaminated variants of the activation domain.

Alternatively, the activation domain may have an amino acid sequence having at least 30% preferably at least 40, 50, 60, 70, 75, 80, 85, 90, 95, 98, or 99% identity with an activation domain listed in Table D6. In a preferred embodiment, the activation domain for use in any of the methods and plants of the present invention is at least 80% identical to the A5 sub-domain of rice RF2a (amino acids 56-84 of SEQ. ID. No. 28).

Exemplary DNA Binding Domains

The DNA-binding domain can comprise, for example, the DNA binding domain of yeast GAL4 protein (“G”), amino acids 1-147) (19), as seen in a VGE or AGE vector (SEQ. ID, No, 29).

(SEQ. ID, No, 29)
MKLLSSIEQACDICRLKKLKCSKEKPKCAKCLKNNWECRYSPKTKRSP
LTRAHLTEVESRLERLEQLFLLIFPREDLDMILKMDSLQDIKALLTGL
FVQDNVNKDAVTDRLASVETDMPLTLRQHRISATSSSEESSNKGQRQL
TVS.

Specific examples of GAL4 based DNA binding domain constructs include, for example, those disclosed in U.S. Pat. No. 5,880,333, U.S. Pat. No. 6,147,282, U.S. Pat. No. 6,939,711, U.S. Pat. No. 5,834,266, U.S. Pat. No. 5,830,462, U.S. Pat. No. 5,834,266, U.S. Pat. No. 5,869,337, U.S. Pat. No. 5,871,753, U.S. Pat. No. 5,994,313, U.S. Pat. No. 6,011,018, U.S. Pat. No. 6,043,082,U.S. Pat. No. 6,046,047, U.S. Pat. No. 6,054,436, U.S. Pat. No. 6,063,625, U.S. Pat. No. 6,140,120, U.S. Pat. No. 6,165,787,U.S. Pat. No. 6,316,418, U.S. Pat. No. 6,891,021,U.S. Pat. No. 6,972,193,U.S. Pat. No. 6,255,558, U.S. Pat. No. 5,968,793 and U.S. Pat. No. 6,958,236.

Similar activation domains from other species may be readily identified by standard homology searching of publicly available databases.

It is well established that the genetic code is degenerate and that some amino acids have multiple codons, and accordingly, multiple polynucleotides can encode the DNA-binding domain used in the trans acting factor. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode the DNA-binding domain can be obtained from by “back-translation” (for example by using Computer programs such as “BackTranslate” (GCG™ Package, Acclerys, Inc. San Diego, Calif.) of the deduced coding sequences derived from the DNA-binding domains of transcription factors identified from genomic clones, from cDNA or EST sequences.

In some embodiments, the DNA-binding domain-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the DNA-binding domain-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the DNA-binding domain-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the GAL4 DNA-binding domain. Polynucleotide “variants” may contain one or more substitutions, additions, deletions and/or insertions in relation to a reference polynucleotide. Generally, variants of the DNA-binding domain reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence as determined by sequence alignment programs described elsewhere herein using default parameters.

In some embodiments the DNA-binding domain which may be used in any of the methods and plants of the invention may have amino acid sequences which are substantially homologous, or substantially similar to SEQ. ID. NO. 29.

For use in the present invention, the DNA-binding domain may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of the DNA-binding domain, are also specifically included in any of the methods of the invention including for example, pyroglutamyl, iso-aspartyl, proteolytic, phosphorylated, glycosylated, reduced, oxidatized, isomerized, and deaminated variants of the DNA-binding domain.

Alternatively, the DNA-binding domain may have an amino acid sequence having at least 30% preferably at least 40, 50, 60, 70, 75, 80, 85, 90, 95, 98, or 99% identity with SEQ. ID. No. 29. In a preferred embodiment, the DNA-binding domain for use in any of the methods and plants of the present invention is at least 80% identical SEQ. ID. No. 29.

### Exemplary Ligand Binding Domains

The ligand binding region can comprise, for example, the ecdysone binding region from the EcR receptor from the spruce budworm Cloristoneura fumiferana ((“E”), amino acids 206-539 of SEQ ID. No. 30 (20) as seen in a VGE or AGE vector.

(SEQ. ID. No. 30)
MRRRWSNNGG FQTLRMLEES SSEVTSSSAL GLPAAMVMSP
ESLASPEYGG LELWGYDDGLSYNTAQSLLG NTCTMQQQQQ
TQPLPSMPLP MPPTTPKSEN ESISSGREEL
SPASSINGCSTDGEARRQKK GPAPRQQEEL CLVCGDRASG
YHYNALTCEG CKGFFRRSVT KNAVYICKFGHACEMDMYMR
RKCQECRLKK CLAVGMRPEC VVPETQCAMK RKEKKAQKEK
DKLPVSTTTVDDHMPPIMQCEPPPPEAARIHEVVPRFLSDKLL
ETNRQKNIPQLTANQQFLIARLIWYQDGYEQPSDEDLKRITQT
WQQADDENEESDTPFRQITEMTILTVQLIVEFAKGLPGFAKIS
QPDQITLLKACSSEVMMLRVARRYDAASDSVLFANNQAYTRDN
YRKAGMAYVIEDLLHFCRCMYSMALDNIHYALLTAVVIFSDRP
GLEQPQLVEEIQRYYLNTLRIYILNQLSGSARSSVIYGKILSI
LS ELRTLGMQNS NMCISLKLKN RKLPPFLEEI
WDVADMSHTQ PPPILESPTNL

Other exemplary EcR ligand binding domains include those of Drosophila melanogaster, Heliothis virescens, and Ostrinia nubilalis and these have been shown to be at least partially interchangeable; (See generally, Tavva et al.; Chapter 21; Ecdysone Receptor-Based Gene Switches for Applications in Plants; G. Smagghe (ed.), Ecdysone: Structures and Functions; © Springer Science+Business Media B.V. 2009). Further ligand binding domains from other species may also be readily identified by standard homology searching of publicly available databases.

It is well established that the genetic code is degenerate and that some amino acids have multiple codons, and accordingly, multiple polynucleotides can encode the ligand binding domain used in the trans acting factor. Moreover, the polynucleotide sequence can be manipulated for various reasons. Examples include, but are not limited to, the incorporation of preferred codons to enhance the expression of the polynucleotide in various organisms (see generally Nakamura et al., Nuc. Acid. Res. (2000) 28 (1): 292). In addition, silent mutations can be incorporated in order to introduce, or eliminate restriction sites, remove cryptic splice sites, or manipulate the ability of single stranded sequences to form stem-loop structures: (see, e.g., Zuker M., Nucl. Acid Res. (2003); 31(13): 3406-3415). In addition, expression can be further optimized by including consensus sequences at and around the start codon.

Such codon optimization can be completed by standard analysis of the preferred codon usage for the host organism in question, and the synthesis of an optimized nucleic acid via standard DNA synthesis. A number of companies provide such services on a fee for services basis and include for example, DNA2.0, (CA, USA) and Operon Technologies. (CA, USA).

In general, non-native nucleic acids that encode the ligand binding domain can be obtained from by “back-translation” (for example by using Computer programs such as “BackTranslate” (GCG™ Package, Acclerys, Inc. San Diego, Calif.) of the deduced coding sequences derived from the DNA-binding domains of transcription factors identified from genomic clones, from cDNA or EST sequences

In some embodiments, the ligand binding domain-encoding nucleotide sequence can designed so that it will be highly expressed in plants. In general, the non-native nucleotide sequence will comprise one or more codons that are more abundant (i.e. occur more frequently) in monocot or dicot plant genes. In certain embodiments, greater than at least 25%, 50%, 70%, 80%, or 90% of the codons used in the ligand binding domain-encoding nucleotide sequence are codons that are more abundant in monocot and/or dicot plant genes. Codon usage in various monocot or dicot genes have been disclosed in Akira Kawabe and Naohiko T. Miyashita. “Patterns of codon usage bias in three dicot and four monocot plant species” Genes Genet. Syst. Vol. 78 343-352 (2003) and E. E. Murray, et al. “Codon Usage in Plant Genes” NAR 17:477-498 (1989).

In certain embodiments, the ligand binding domain-encoding nucleotide sequence can be obtained using one or more methods that have been previously described. U.S. Pat. No. 5,500,365 describes a method for synthesizing plant genes to optimize the expression level of the protein encoded by the synthesized gene. This method relates to the modification of the structural gene sequences of the exogenous transgene, to make them more “plant-like” and therefore more efficiently transcribed, processed, translated and expressed by the plant. Features of genes that are expressed well in plants include use of codons that are commonly used by the plant host and elimination of sequences that can cause undesired intron splicing or polyadenylation in the coding region of a gene transcript.

A similar method for obtaining enhanced expression of transgenes in monocotyledonous plants is disclosed in U.S. Pat. No. 5,689,052. Furthermore, the synthetic design methods disclosed in U.S. Pat. No. 5,500,365 and U.S. Pat. No. 5,689,052 could also be used to synthesize a signal peptide encoding sequence that is optimized for expression in plants in general or monocot plants in particular.

Embodiments of the present invention also include “variants” of the ligand binding domain. Polynucleotide “variants” may contain one or more substitutions, additions, deletions and/or insertions in relation to a reference polynucleotide. Generally, variants of the ligand binding domain reference polynucleotide sequence may have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence as determined by sequence alignment programs described elsewhere herein using default parameters.

In some embodiments the ligand binding domain which may be used in any of the methods and plants of the invention may have amino acid sequences which are substantially homologous, or substantially similar to amino acids 206-539 of SEQ. ID. NO. 30.

For use in the present invention, the ligand binding domain may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, substitutions.

Naturally-occurring chemical modifications including post-translational modifications and degradation products of the ligand binding domain, are also specifically included in any of the methods of the invention including for example, pyroglutamyl, iso-aspartyl, proteolytic, phosphorylated, glycosylated, reduced, oxidatized, isomerized, and deaminated variants of the ligand binding domain.

Alternatively, the ligand binding domain may have an amino acid sequence having at least 30% preferably at least 40, 50, 60, 70, 75, 80, 85, 90, 95, 98, or 99% identity with SEQ. ID. No. 30. In a preferred embodiment, the ligand binding domain for use in any of the methods and plants of the present invention is at least 80% identical SEQ. ID. No. 30.

In one embodiment, the EcR binds a non-steroidal ecdysone modulator, for example, a tubefenozide, a Methoxyfenozide, a diacylhydrazine, and the like.

Cis-Element

A cis-element of the present invention is any element that, when operably linked to a polynucleotide, allows transcription of the polynucleotide to be modulated by the binding of a trans-acting factor to the cis-element. Cis-elements may be identified in chemically-regulated genes, for example, by a binding assay with the appropriate chemical modifier.

In one embodiment, the cis-element is an ecdysone response element (EcRE). In one embodiment, the cis element is a GAL4 response element (UAS) (5′-CGGRNNRCYNYNCNCCG-3′ SEQ. ID. No. 31). In one aspect the cis element comprises several tandemly repeated copies of the GAL4 UAS. In some embodiments, the cis-element comprises 5 copies of the GAL4 response element. Other useful cis-elements include those which are regulated by ecdysone modulators, tetracyclines, steroids, glucocorticoids, estradiols, salicylic acid, or ethanol.

Exemplary Minimal Promoters

In one embodiment of the present invention, a hormone-regulating gene is operably linked to a minimal promoter comprising a cis-element capable of binding the trans-acting factor.

The promoter may be provided, for example, by fusing a cis-element (e.g. multiple copies of a cis acting element) to a known promoter sequence (e.g. a minimal promoter such as truncated 35S promoter (Padidam et al., (2003) Transgenic. Res. 12 101-109). A number of promoters which are useful in gene switches are known in the art.

In some embodiments of the gene switch the promoter (e.g. a minimal promoter or a promoter fused to one or more cis-elements) may be constitutive (e.g. 35S promoter) or tissue-specific (e.g. NCED promoter). Other useful promoters include promoters of seed storage proteins and seed maturation-associated genes, such as LEA proteins (e.g. AtEm1 and AtEM6), which are stage-specific.

In one embodiment, the promoter comprises a seed-specific promoter fused to the cis element.

In one embodiment, the promoter comprises an endosperm-specific promoter fused to the cis element.

In one embodiment, the promoter comprises at least one ecdysone response element (EcRE) as the cis-element.

In one embodiment, the promoter comprises at least one GAL4 response element as part of the cis-element. In one aspect, the cis element comprises at least 5 copies of the GAL4 response element.

Exemplary Chemical Modifiers

A chemical modulator of the present invention is an agent that interacts with a trans-acting factor, thereby modulating the regulatory activity of the trans-acting factor on the cis-element and thereby modulates the transcription of an operably linked polynucleotide. In some embodiments the chemical modifier is a ligand of the ligand binding domain.

Useful chemical modulators of the present invention may have one or more of the following characteristics: bio-safe, degradable, non-naturally occurring in the microenvironment of the photosynthetic organism, active in low, commercially feasible concentrations; and/or easy to administer.

The chemical modulator may be positive modulator (e.g. inducer or agonist) or a negative modulator (e.g. repressor or antagonist). In some embodiments, the chemical modulator is a positive modulator and induces the functionality of the trans-acting factor with respect to the cis-element. In some embodiments, the chemical modulator is a negative modulator and suppresses the functionality of the trans-acting factor with respect to the cis-element. A chemical modulator may, for example, induce or inhibit a conformational change in a trans-acting factor.

In one embodiment, the chemical modulator is an ecdysone modulator. In one embodiment, the chemical modulator is a non-steroidal ecdysone modulator. A number of ecdysone modulators are known in the art. Optionally, ecdysone modulator is a tubefenozide, or a Methoxyfenozide. Optionally, the ecdysone modulator is a diacylhydrazine.

### Administration of Chemical Modulators

Surprisingly, in one embodiment, it is now possible to control germination at will by administering (or withholding) a chemical modulator (e.g. chemical inducer).

The present invention contemplates any method of administration that results (directly or indirectly) in contacting a chemical modulator with a trans-acting factor such that transcription of a polynucleotide operably linked to the cis-element is modulated. The method of administration may be active or may be passive.

A chemical modulator may be administered, for example, by “drenching.” A transgenic plant seed may be drenched in an inducer, for example, by any of the following Methods: direct imbibition in the presence of an inducer or pre-germination treatment with an inducer, such as seed priming, pelleting or film coating.

Other useful examples of administration Methods include drenching developing plants or treating siliques (pods) with an inducer.

### Physical Insult of Seeds

In one embodiment, a seed is physically insulted to permeabilize an outer layer thereof (e.g. coat). Such permeabilize optionally enhances hydration or uptake of a chemical modulator. Physical insult such as scarification, mechanical insult, and chemical insult are known in the art, for example, as described by Burns (“Effect Of Acid Scarification On Lupine Seed Impermeability”; Plant Physiol. 1959 March; 34(2): 107-108) and (“Seed Anatomy and Water Uptake in Relation to Seed Dormancy in Opuntia tomentosa (Cactaceae, Opuntioideae”; Annals of Botany 99: 581-592, 2007).

In one embodiment, the physical insult comprises scarification.

In one embodiment, the physical insult comprises mechanical or chemical (e.g. acid) treatment.

In one embodiment, a seed transformed with a germination metabolizing hormone operably linked to a chemically inducible promoter is physically insulted. Optionally, germination metabolizing hormone is a seed germination gene.

### Hosts

With the present invention, it is now possible to use chemical modulators to control seed germination in a host plant. The host plant may be any plant.

Useful hosts include crop plants (for example, cereals and pulses, maize, wheat, potatoes, tapioca, rice, sorghum, millet, cassaya, barley, pea, and other root, tuber, or seed crops. Examples of useful seed crops include oil-seed rape, false flax (Camelina sativa), sugar beet, maize, sunflower, soybean, and sorghum.

Other useful hosts include vegetables and flowers for which F-1 hybrids are available and the protection of intellectual property is an issue.

### Germination Control

According to the present invention, seed germination can now be controlled by transforming a plant with one or more hormone-regulating genes taught herein. The one or more hormone-regulating genes can be, for example, seed-germination genes or seed-dormancy genes. Seed germination can now be controlled relative to a control host (e.g. a host not transformed with one or more hormone-regulating genes according to the present invention), for example, even under unfavorable conditions.

In one embodiment, germination control comprises suppressing seed germination or inducing seed dormancy. Such germination control can be imparted by transforming a plant with a seed dormancy gene, as taught herein.

In one embodiment, germination control comprises suppressing seed dormancy or inducing seed germination. Such germination control can be imparted by transforming a plant with a seed germination gene, as taught herein.

In one embodiment, the seed germination which is suppressed comprises precocious germination, for example, pre-harvest sprouting (PHS). Optionally, the plant is a cereal crop, such as wheat or barley. PHS can dramatically reduce crop quality since precocious germination prematurely triggers mobilization of starch, which should optimally ally occur only after germination.

In one embodiment, the seed germination which is suppressed is the seed germination of a biofuel crop such as Camelina (e.g. C. sativa).

In one embodiment, the seed dormancy which is suppressed is primary or secondary (e.g. high-temperature induced) dormancy. Seeds that are released from the plant in a dormant state are said to exhibit primary dormancy. Seeds that are released from the plant (e.g. in a nondormant state) but which become dormant if the conditions for germination are unfavorable exhibit secondary dormancy. For example, seeds of Avena sativa (oat) can become dormant in the presence of temperatures higher than the maximum for germination, whereas seeds of Phacelia dubia (small-flower scorpionweed) become dormant at temperatures below the minimum for germination.

In one embodiment, the seed dormancy which is suppressed is high-temperature induced dormancy. Optionally, the plant is a vegetable crop such as lettuce. Optionally, the plant is barley. High-temperature induced dormancy and conditions which normally induce such dormancy (e.g. in a control plant) are known in the art and described, for example, by Toh et al. (“High Temperature-Induced Abscisic Acid Biosynthesis and Its Role in the Inhibition of Gibberellin Action in Arabidopsis Seeds”; Plant Physiology, March 2008, Vol. 146, pp. 1368-1385), Leymarie et al. (“Involvement of ABA in Induction of Secondary Dormancy in Barley (Hordeum vulgare L.) Seeds”; Plant Cell Physiol. 49(12): 1830-1838 (2008)), Kristie et al. (“Factors Affecting the Induction of Secondary Dormancy in Lettuce”; Plant Physiol. (1981) 67, 1224-1229), and Argyris et al. (“Genetic Variation for Lettuce Seed Thermoinhibition Is Associated with Temperature-Sensitive Expression of Abscisic Acid, Gibberellin, and Ethylene Biosynthesis, Mabolism, and Response Genes”; Plant Physiology, October 2008, Vol. 148, pp. 926-947). Typically, high-temperature induced dormancy comprises incubating a seed under a temperature greater than that which a seed can germinate.

### Transformation

Techniques for transforming a wide variety of plant species are well known and described in the technical and scientific literature. See, for example, Weising et al, (1988) Ann. Rev. Genet., 22:421-477. As described herein, the DNA constructs of the present invention typically contain a marker gene which confers a selectable phenotype on the plant cells. For example, the marker may encode biocide resistance, particularly antibiotic resistance, such as resistance to kanamycin, G418, bleomycin, hygromycin, or herbicide resistance, such as resistance to chlorsulfuron or Basta. Such selective marker genes are useful in protocols for the production of transgenic plants.

DNA constructs can be introduced into the genome of the desired plant host by a variety of conventional techniques. For example, the DNA construct may be introduced directly into the plant cell using techniques such as electroporation and microinjection of plant cell protoplasts. Alternatively, the DNA constructs can be introduced directly to plant tissue using biolistic methods, such as DNA micro-particle bombardment. In addition, the DNA constructs may be combined with suitable transfer DNA (T-DNA) flanking regions and introduced into a conventional Agrobacterium tumefaciens Ti Plasmid. The T-DNA of the Ti plasmid will be transferred into plant cell through Agrobacterium-mediated transformation system.

Microinjection techniques are known in the art and well described in the scientific and patent literature. The introduction of DNA constructs using polyethylene glycol precipitation is described in Paszkowski et al, (1984) EMBO J., 3:2717-2722. Electroporation techniques are described in Fromm et al, (1985) Proc. Natl. Acad. Sci. USA, 82:5824. Biolistic transformation techniques are described in Klein et al, (1987) Nature 327:70-7. The full disclosures of all references cited are incorporated herein by reference.

A variation involves high velocity biolistic penetration by small particles with the nucleic acid either within the matrix of small beads or particles, or on the surface (Klein et al., (1987), Nature, 327:70-73). Although typically only a single introduction of a new nucleic acid segment is required, this method particularly provides for multiple introductions.

Agrobacterium tumefaciens-meditated transformation techniques are well described in the scientific literature. See, for example Horsch et al, (1984) Science, 233:496-498, and Fraley et al, (1983) Proc. Natl. Acad. Sci. USA, 90:4803.

More specifically, a plant cell, an explant, a meristem or a seed is infected with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., (1984), Science, 233:496-498; Fraley et al., (1983), Proc. Nat'l. Acad. Sci. U.S.A., 80:4803.

Ti plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constructs of the invention into an appropriate plant cell, such being a “disabled Ti vector”.

All plant cells which can be transformed by Agrobacterium and whole plants regenerated from the transformed cells can also be transformed according to the invention so as to produce transformed whole plants which contain the transferred foreign nucleic acid sequence. There are various ways to transform plant cells with Agrobacterium, including: (1) co-cultivation of Agrobacterium with cultured isolated protoplasts, (2) co-cultivation of cells or tissues with Agrobacterium, or (3) transformation of developing embryos, leaves, apices, or meristems with Agrobacterium.

Method (1) requires an established culture system that allows culturing protoplasts and plant regeneration from cultured protoplasts. Method (2) requires (a) that the plant cells or tissues can be transformed by Agrobacterium and (b) that the transformed cells or tissues can be induced to regenerate into whole plants. Method (3) requires micropropagation.

In the binary system, to have infection, two plasmids are needed: a T-DNA containing plasmid and a vir plasmid. Any one of a number of T-DNA containing plasmids can be used, the only requirement is that one be able to select independently for each of the two plasmids. After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance or visual observation. Other phenotypic markers are known in the art and may be used in this invention.

The present invention embraces use of the claimed modified hemA constructs in transformation of any plant, including both dicots and monocots. Transformation of dicots is described in references above. Transformation of monocots is known using various techniques including electroporation (e.g., Shimamoto et al., (1992), Nature, 338:274-276); ballistics (e.g., European Patent Application 270,356); and Agrobacterium (e.g., Bytebier et al., (1987), Proc. Nat'l Acad. Sci. USA, 84:5345-5349).

Transformed plant cells which are derived by any of the above transformation techniques can be cultured to regenerate a whole plant which possesses the desired transformed phenotype. Such regeneration techniques rely on manipulation of certain phytohormones in a tissue culture growth medium typically relying on a biocide and/or herbicide marker which has been introduced together with the nucleotide sequences. Plant regeneration from cultured protoplasts is described in Evans et al, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally by Klee et al, (1987) Ann. Rev. Plant Phys., 38:467-486. Additional methods for producing a transgenic plant useful in the present invention are described in U.S. Pat. Nos. 5,188,642; 5,202,422; 5,384,253; 5,463,175; and 5,639,947. The methods, compositions, and expression vectors of the invention have use over a broad range of types of plants, including the creation of transgenic plant species belonging to virtually any species including for example, canola, camelina, flax, alfalfa, soybean, cotton, corn, rice, wheat, barley and etc.

Selection

Typically DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin, G418 and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase (hpt).

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One example of an herbicide which is useful for selection of transformed cell lines in the practice of the invention is the broad spectrum herbicide glyphosate. Glyphosate inhibits the action of the enzyme EPSPS, which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, PCT Publication WO 97/04103. The best characterized mutant EPSPS gene conferring glyphosate resistance comprises amino acid changes at residues 102 and 106, although it is anticipated that other mutations will also be useful (PCT Publication WO 97/04103). Furthermore, a naturally occurring glyphosate resistant EPSPS may be used, e.g., the CP4 gene isolated from Agrobacterium encodes a glyphosate resistant EPSPS (U.S. Pat. No. 5,627,061).

To use the bar-bialaphos or the EPSPS-glyphosate selective systems, tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is believed that ranges of 0.1-50 mg/l bialaphos or 0.1-50 mM glyphosate will find utility in the practice of the invention. Bialaphos and glyphosate are provided as examples of agents suitable for selection of transformants, but the technique of this invention is not limited to them.

Another herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism. Synthetic PPT, the active ingredient in the herbicide LIBERTY™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity. The bar gene has been cloned and expressed in transgenic tobacco, tomato, potato, Brassica and maize (U.S. Pat. No. 5,550,318). In previous reports, some transgenic plants which expressed the resistance gene were completely resistant to commercial formulations of PPT and bialaphos in greenhouses.

It further is contemplated that the herbicide dalapon, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (U.S. Pat. No. 5,780,708).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468 and U.S. Pat. No. 6,118,047.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells that are expressing luciferase and manipulate cells expressing in real time. Another screenable marker which may be used in a similar fashion is the gene coding for green fluorescent protein (GFP) or a gene coding for other fluorescing proteins such as DSRED® (Clontech, Palo Alto, Calif.).

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase or GFP would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene (WO 99/60129).

### Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. Preferred growth regulators for plant regeneration include cytokins such as 6-benzylamino pierine, zeahin or the like, and abscisic acid. Media improvement in these and like ways has been found to facilitate the growth of cells at specific developmental stages. Tissue may be maintained on a basic media with auxin type growth regulators until sufficient tissue is available to begin plant regeneration efforts, or following repeated rounds of manual selection, until the morphology of the tissue is suitable for regeneration, then transferred to media conducive to maturation of embryoids. Cultures are transferred every 1-4 weeks, preferably every 2-3 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets were transferred to soilless plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous expressible gene. Note however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Characterization

To confirm the presence of the exogenous DNA or “transgene(s)” in the regenerating plants, a variety of assays, known in the art may be performed. Such assays include, for example, “molecular biological” assays, such as Southern and Northern blotting and PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR). Using this technique discreet fragments of DNA are amplified and detected by gel electrophoresis. This type of analysis permits one to determine whether a gene is present in a stable transformant, but does not necessarily prove integration of the introduced gene into the host cell genome. Typically, DNA has been integrated into the genome of all transformants that demonstrate the presence of the gene through PCR analysis. In addition, it is not possible using PCR techniques to determine whether transformants have exogenous genes introduced into different sites in the genome, i.e., whether transformants are of independent origin. Using PCR techniques it is possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition, it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

It is contemplated that using the techniques of dot or slot blot hybridization, which are modifications of Southern hybridization techniques, one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR techniques, referred to as RT-PCR, also may be used for detection and quantification of RNA produced from introduced genes. In this application of PCR it is first necessary to reverse transcribe RNA into DNA, using enzymes such as reverse transcriptase, and then through the use of conventional PCR techniques amplify the DNA. In most instances PC techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

It is further contemplated that TAQMAN® technology (Applied Biosystems, Foster City, Calif.) may be used to quantitate both DNA and RNA in a transgenic cell.

Gene Expression

While Southern blotting and PCR may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following an increase in fluorescence as anthranilate is produced, to name two.

Very frequently the expression of a gene product is determined by evaluating the phenotypic results of its expression. These assays also may take many forms, including but not limited to, analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

Event Specific Transgene Assay

Southern blotting, PCR and RT-PCR techniques can be used to identify the presence or absence of a given transgene but, depending upon experimental design, may not specifically and uniquely identify identical or related transgene constructs located at different insertion points within the recipient genome. To more precisely characterize the presence of transgenic material in a transformed plant, one skilled in the art could identify the point of insertion of the transgene and, using the sequence of the recipient genome flanking the transgene, develop an assay that specifically and uniquely identifies a particular insertion event. Many methods can be used to determine the point of insertion such as, but not limited to, Genome Walker™ technology (CLONTECH, Palo Alto, Calif.), Vectorette™ technology (Sigma, St. Louis, Mo.), restriction site oligonucleotide PCR, uneven PCR (Chen and Wu, (1997), Gene, 185: 195-1199) and generation of genomic DNA clones containing the transgene of interest in a vector such as, but not limited to, lambda phage.

Once the sequence of the genomic DNA directly adjacent to the transgenic insert on either or both sides has been determined, one skilled in the art can develop an assay to specifically and uniquely identify the insertion event. For example, two oligonucleotide primers can be designed, one wholly contained within the transgene and one wholly contained within the flanking sequence, which can be used together with the PCR technique to generate a PCR product unique to the inserted transgene. In one embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the transgene. In another embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the genomic sequence adjacent to the insertion site. Confirmation of the PCR reaction may be monitored by, but not limited to, size analysis on gel electrophoresis, sequence analysis, hybridization of the PCR product to a specific radiolabeled DNA or RNA probe or to a molecular beacon, or use of the primers in conjugation with a TAQMAN™ probe and technology (Applied Biosystems, Foster City, Calif.).

Site Specific Integration or Excision of DNA Sequences

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. This random insertion of introduced DNA into the genome of host cells can be detrimental to the cell if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence. Furthermore, site-specific integration or excision offers a means to create a mutated gene of interest by adding or deleting sequences as designed for example to modify a hemA gene in a plant species of interest.

Site-specific integration can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. Thus random integration of transgenes is more common in plants. To maintain control over the copy number and the location of the inserted DNA, randomly inserted DNA sequences can be removed. One manner of removing these random insertions is to utilize a site-specific recombinase system (U.S. Pat. No. 5,527,695).

A recently invented synthetic zinc finger nucleases (ZFNs) technology provides a powerful tool to modify the genome of given species by adding or deleting DNA sequences. ZFNs function as dimers with each monomer composed of a synthetic zinc finger domain fused with a nonspecific cleavage domain of the Fokl endonuclease. The zinc finger domain in each of the monomers recognizes and binds to specific sequences in the genome as designed, typically 18 or 24 bp depending on the number of zinc fingers in the synthetic zinc finger domain. Two ZFN monomer recognition sites are spaced by 5 to 7 bp. The zinc finger domain in the ZFN monomers will direct the Fokl to the two adjacent DNA recognition sites of the ZFN monomers, form a functional Fokl dimer and generate a DNA double-strand break (DSB) in the spacer sequence between the two zinc finger recognition sites (Zhang et al., (2010), Proc. Nat'l Acad. Sci. USA 107:12028-1203; Cui et al. (2011), Nature Biotechnology 29: 64-68). During the process of repairing chromosome breaks, nonhomologous end-joining or homologous recombination will occur which will greatly enhance the frequencies of targeted integration or deletion of DNA sequences. This method has been demonstrated very effective in Arabidopsis (Zhang et al., (2010) PNAS 107:12028-1203) and can be employed to create mutants of the hormone-regulating gene in a plant species of interest.

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast, the Gin recombinase of phage Mu, the Pin recombinase of E. coli, and the R/RS system of the pSRi plasmid. The bacteriophage P1 Cre/lox and the yeast FLP/FRT systems constitute two particularly useful systems for site specific integration or excision of transgenes. In these systems, a recombinase (Cre or FLP) will interact specifically with its respective site-specific recombination sequence (lox or FRT, respectively) to invert or excise the intervening sequences. The sequence for each of these two systems is relatively short (34 bp for lox and 47 bp for FRT) and therefore, convenient for use with transformation vectors.

The FLP/FRT recombinase system has been demonstrated to function efficiently in plant cells. Experiments on the performance of the FLP/FRT system in both maize and rice protoplasts indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between lox sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, (1987), Mol. Cell. Biol. 7:2087-2096). Cre was expressed from an inducible yeast GAL™ promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

Deletion of Sequences Located within the Transgenic Insert

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

It is known that homologous recombination results in genetic rearrangements of transgenes in plants. Repeated DNA sequences have been shown to lead to deletion of a flanked sequence in various dicot species, e.g. Arabidopsis thaliana and Nicotiana tabacum. One of the most widely held models for homologous recombination is the double-strand break repair (DSBR) model.

Deletion of sequences by homologous recombination relies upon directly repeated DNA sequences positioned about the region to be excised in which the repeated DNA sequences direct excision utilizing native cellular recombination mechanisms. The first fertile transgenic plants are crossed to produce either hybrid or inbred progeny plants, and from those progeny plants, one or more second fertile transgenic plants are selected which contain a second DNA sequence that has been altered by recombination, preferably resulting in the deletion of the ancillary sequence. The first fertile plant can be either hemizygous or homozygous for the DNA sequence containing the directly repeated DNA which will drive the recombination event.

The directly repeated sequences are located 5′ and 3′ to the target sequence in the transgene. As a result of the recombination event, the transgene target sequence may be deleted, amplified or otherwise modified within the plant genome. In the preferred embodiment, a deletion of the target sequence flanked by the directly repeated sequence will result.

Alternatively, directly repeated DNA sequence mediated alterations of transgene insertions may be produced in somatic cells. Preferably, recombination occurs in a cultured cell, e.g., callus, and may be selected based on deletion of a negative selectable marker gene, e.g., the periA gene isolated from Burkholderia caryolphilli which encodes a phosphonate ester hydrolase enzyme that catalyzes the hydrolysis of glyceryl glyphosate to the toxic compound glyphosate (U.S. Pat. No. 5,254,801).

Vectors and Constructs

In one aspect, vectors are provided for transforming a plant according to the present invention.

One embodiment of the present invention provides plant transformed with a hormone-regulating gene operably linked to a promoter.

One embodiment of the present invention provides plant transformed with a seed dormancy gene operably linked to a first promoter and a seed germination gene operably linked to a second promoter.

One embodiment of the present invention provides a plant transformed with 1) a first genetic construct comprising a first hormone-regulating gene operably linked to a promoter comprising a cis-element regulated by the activity of a trans-acting factor; 2) a second genetic construct comprising an expressible gene encoding the trans-acting factor. Optionally, the plant further comprises a third genetic construct comprising a second hormone regulating gene operably linked to a second promoter (e.g. a spontaneous promoter such a seed specific or endosperm specific promoter). For example, the first hormone-regulating gene can be a seed germination gene and the second gene can be a seed dormancy gene (e.g. NCED6).

In one embodiment, the present invention provides a vector comprising the first genetic construct. Optionally, the vector (or collection of vectors) further comprises the second genetic construct. Optionally, the vector (or collection of vectors) comprises the third genetic construct.

In one embodiment, the present invention provides a vector for creating the first genetic construct in the host.

Expression vectors suitable for use in expressing the claimed DNA constructs in plants, and methods for their construction are generally well known, and need not be limited. These techniques, including techniques for nucleic acid manipulation of genes such as subcloning a subject promoter, or nucleic acid sequences encoding a gene of interest into expression vectors, labeling probes, DNA hybridization, and the like, and are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. For instance, various procedures, such as PCR, or site directed mutagenesis can be used to introduce a restriction site at the start codon of a heterologous gene of interest. Heterologous DNA sequences are then linked to a suitable expression control sequences such that the expression of the gene of interest are regulated (operatively coupled) by the promoter.

DNA constructs comprising an expression cassette for the gene of interest can then be inserted into a variety of expression vectors. Such vectors include expression vectors that are useful in the transformation of plant Cells. Many other such vectors useful in the transformation of plant cells can be constructed by the use of recombinant DNA techniques well known to those of skill in the art as described above.

Exemplary expression vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein.

Exemplary vectors for expression using Agrobacterium tumefaciens-mediated plant transformation include for example, pBin 19 (CLONETECH), Frisch et al, Plant Mol. Biol., 27:405-409, 1995; pCAMBIA 1200 and pCAMBIA 1201 (Center for the Application of Molecular Biology to International Agriculture, Canberra, Australia); pGA482, An et al, (1985), EMBO J., 4:277-284; pCGN1547, (CALGENE Inc.) McBride et al, (1990), Plant Mol. Biol., 14:269-276, and the like vectors, such as is described herein.

Promoters.

DNA constructs will typically include promoters to drive expression of the gene switch construct, or hormone-regulating gene of interest. Promoters may provide ubiquitous, cell type specific, constitutive promoter or inducible promoter expression. Basal promoters in plants typically comprise canonical regions associated with the initiation of transcription, such as CAAT and TATA boxes. The TATA box element is usually located approximately 20 to 35 nucleotides upstream of the initiation site of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is, regions comprising sequences from the CAAT box to the translational start site, with other upstream promoter elements to enhance or otherwise alter promoter activity or specificity.

In some aspects promoters may be altered to contain “enhancer DNA” to assist in elevating gene expression. As is known in the art certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are particularly useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (U.S. Pat. No. 5,593,874), the maize shrunken 1 gene, the light sensitive 1 gene of Solanum tuberosum, and the heat shock protein 70 gene of Petunia hybrida (U.S. Pat. No. 5,659,122).

For in vivo expression in plants, exemplary constitutive promoters include those derived from the CaMV 35S, rice actin, Cassaya vein mosaic virus promoter, fig mosaic virus promoter, Nos promoter, tubulin promoter, and maize ubiquitin genes. Exemplary inducible promoters for this purpose include the chemically inducible PR-1a promoter the Ecdysone inducible promoter, the ethanol inducible promoter, dexamethasone-inducible promoter, methoxyfenozide inducible promoter, ABA inducible promoter. Selected promoters can direct expression in specific cell types (such as leaf epidermal cells, mesophyll cells, root cortex cells) or in specific tissues or organs (roots, leaves, flowers or embryos, for example). Exemplary tissue or developmentally specific promoters include well-characterized root-, pith-, leaf-, and embryo-specific promoters, each described herein below.

Depending upon the host cell system utilized, any one of a number of suitable promoters can be used. Promoter selection can be based on expression profile and expression level. The following are representative non-limiting examples of promoters that can be used in the expression cassettes.

35S Promoter.

The CaMV 35S promoter can be used to drive constitutive gene expression. Construction of the plasmid pCGN1761 is described in the published patent application EP 0 392 225, which a CaMV 35S promoter and the tml transcriptional terminator with a unique EcoRI site between the promoter and the terminator and has a pUC-type backbone.

Actin Promoter.

Several isoforms of actin are known to be expressed in most cell types and consequently the actin promoter is a good choice for a constitutive promoter. In particular, the promoter from the rice Act/gene has been cloned and characterized (McElroy et al., 1990). A 1.3 kb fragment of the promoter was found to contain inter ali the regulatory elements required for expression in rice protoplasts. Furthermore, numerous expression vectors based on the Act/promoter have been constructed specifically for use in monocotyledons (McElroy et al., 1991). These incorporate the Act/-intron 1, Adbl 5′ flanking sequence and Adbl-intron 1 (from the maize alcohol dehydrogenase gene) and sequence from the CaMV 35S promoter. Vectors showing highest expression were fusions of 35S and Act/intron or the Act/5′ flanking sequence and the AcV intron. Optimization of sequences around the initiating ATG (of the GUS reporter gene) also enhanced expression.

Ubiquitin Promoter.

Ubiquitin is another gene product known to accumulate in many cell types and its promoter has been cloned from several species for use in transgenic plants (e.g. sunflower, and maize). The maize ubiquitin promoter has been developed in transgenic monocot systems and its sequence and vectors constructed for monocot transformation are disclosed in the patent publication EP 0 342 926 which is herein incorporated by reference. The ubiquitin promoter is suitable for gene expression in transgenic plants, especially monocotyledons. Suitable vectors include derivatives of pAHC25, or any of the transformation vectors described in this application, modified by the introduction of the appropriate ubiquitin promoter and/or intron sequences.

Exemplary developmentally specific, and cell type specific promoters, include root specific, leaf specific, pith specific, seed specific, germination and embryogenesis specific promoters. Representative examples of such promoters include for example, the patatin promoter (Topfer et al., (1989), Mol Gen Genet., 219:390-396), chlorophyll a/b-binding protein promoters (Mitra et al, (1989), Plant Molecular Biology, 12: 169-179), the beta conglycinin promoter (Allen et al., (1989), Plant Cell, 1: 623-631), the oleosin promoter (Plant et al., (1994), Plant Molecular Biology, 12: 169-179), and glycinin promoter (Itoh et al., (1993), Plant Molecular Biology, 21: 973-84).

In one embodiment, a promoter comprises a seed-specific expression control sequence.

In one embodiment, a promoter comprises an endosperm-specific.

The promoter of choice is preferably excised from its source by restriction enzymes, but can alternatively be PCR-amplified using primers that carry appropriate terminal restriction sites. The selected target gene coding sequence can be inserted into this vector, and the fusion products (i.e., promoter-gene-terminator) can subsequently be transferred to any selected transformation vector, including those described below.

Transcriptional Terminators

A variety of transcriptional terminators are available for use in the DNA constructs of the invention. These are responsible for the termination of transcription beyond the transgene and its correct polyadenylation.

Appropriate transcriptional terminators are those that are known to function in the relevant microalgae or plant system. Representative plant transcriptional terminators include the CaMV 35S terminator, the tml terminator, the nopaline synthase terminator, and the pea rbcS E9 terminator. With regard to RNA polymerase III terminators, these terminators typically comprise a −52 run of 5 or more consecutive thymidine residues. In one embodiment, an RNA polymerase III terminator comprises the sequence TTTTTTT. These can be used in both monocotyledons and dicotyledons.

Sequences for the Enhancement or Regulation of Expression

Numerous sequences have been found to enhance the expression of an operatively lined nucleic acid sequence, and these sequences can be used in conjunction with the nucleic acids of the presently disclosed subject matter to increase their expression in transgenic plants.

Various intron sequences have been shown to enhance expression, particularly in monocotyledonous cells. For example, the introns of the maize Adbl gene have been found to significantly enhance the expression of the wild-type gene under its cognate promoter when introduced into maize cells. Intron 1 was found to be particularly effective and enhanced expression in fusion constructs with the chloramphenicol acetyltransferase gene. In the same experimental system, the intron from the maize bronzes gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMY) have been shown to be effective in enhancing expression.

Selectable Markers:

For certain target species, different antibiotic or herbicide selection markers can be included in the DNA constructs of the invention. Selection markers used routinely in transformation include the nptII gene, which confers resistance to kanamycin and related antibiotics, the bar gene, which confers resistance to the herbicide phosphinothricin, the hph gene, which confers resistance to the antibiotic hygromycin, the dhfr gene, which confers resistance to methotrexate, and the EPSP synthase gene, which confers resistance to glyphosate (U.S. Pat. Nos. 4,940,935 and 5,188,642).

Screenable Markers

Screenable markers may also be employed in the DNA constructs of the present invention, including for example the β-glucuronidase or uidA gene (the protein product is commonly referred to as GUS), isolated from E. coli, which encodes an enzyme for which various chromogenic substrates are known; an R-locus gene, which encodes a product that regulates the production of anthocyanin pigments (red color) in plant tissues; a β-lactamase gene, which encodes an enzyme for which various chromogenic substrates are known (e.g., PADAC, a chromogenic cephalosporin); a xy/E gene, which encodes a catechol dioxygenase that can convert chromogenic catechols; an α-amylase gene; a tyrosinase gene which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene, which allows for bioluminescence detection; an aequorin gene, which may be employed in calcium-sensitive bioluminescence detection; or a gene encoding for green fluorescent protein (PCT Publication WO 97/41228).

The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which has the genotype r-g, b, Pl. Alternatively, any genotype of maize can be utilized if the C1 and R alleles are introduced together.

In some aspects, screenable markers provide for visible light emission as a screenable phenotype. A screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for population screening for bioluminescence, such as on tissue culture plates, or even for whole plant screening. The gene which encodes green fluorescent protein (GFP) is contemplated as a particularly useful reporter gene (PCT Publication WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP-NPTII gene fusion (PCT Publication WO 99/60129). This could allow, for example, selection of transformed cells followed by screening of transgenic plants or seeds. In a similar manner, it is possible to utilize other readily available fluorescent proteins such as red fluorescent protein (CLONTECH, Palo Alto, Calif.).

Transgenic Organisms

In one aspect the invention also contemplates a transgenic plant comprising:
i) a first nucleic acid sequence comprising a first polynucleotide sequence encoding a trans-acting factor; wherein the trans-acting factor is controlled by a chemical modulator and
ii) a second nucleic acid sequence comprising a cis-element capable of binding the trans-acting factor operatively coupled to a first hormone-regulating gene.

In some embodiments, the current invention includes a transgenic organism further comprising:

i) a third nucleic acid sequence comprising a second polynucleotide sequence encoding a second hormone-regulating gene coupled to a seed specific promoter.

In some embodiments the first hormone regulating gene is selected from Table D1. In one aspect the first hormone regulating gene is at least 80% identical to SEQ. ID. No. 7.

In some embodiments the first hormone regulating gene is selected from Table D2. In one aspect the first hormone regulating gene is at least 80% identical to any of SEQ. ID. Nos. 1, 2 or 7. In some embodiments the seed specific promoter is an NCED6 promoter.

In some aspects the first and second nucleic acid sequences are present on the same genetic construct.

In some aspects, the trans acting factor comprises an activation domain from RF2a, operatively coupled to a GAL4 DNA binding domain which is operatively coupled to an Ecdysone receptor ligand binding domain.

### EXAMPLES

### Overview of Plant Gene Switch System (PGSS)

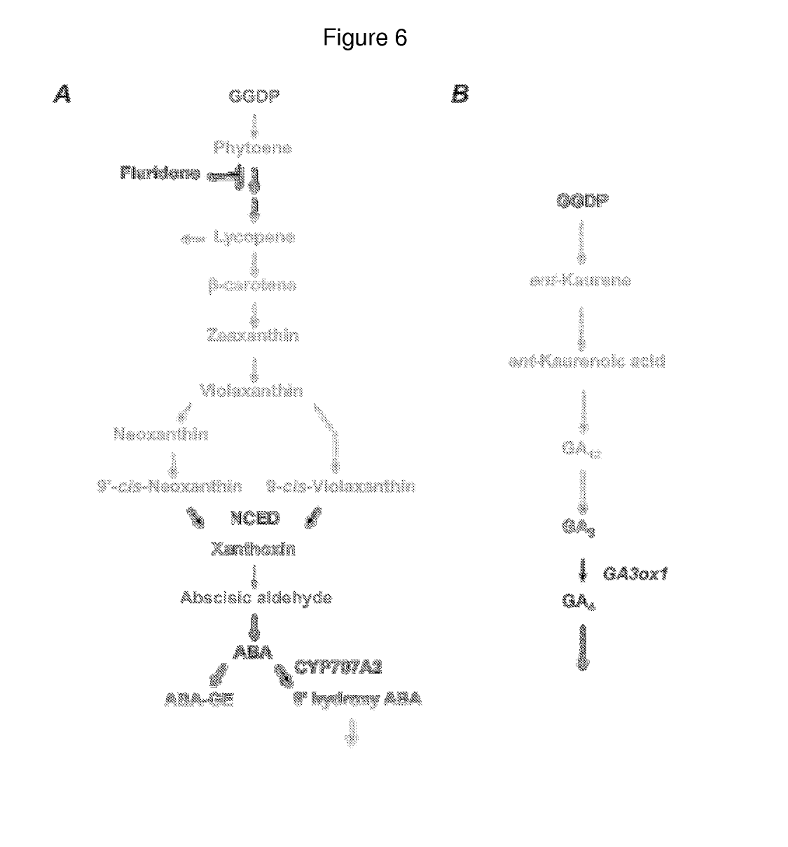

The inventors speculated that one or more rate-limiting ABA biosynthesis genes (FIG. 6) could be placed under the control of a Plant Gene Switch System (PGSS), a chemically inducible gene expression system, to alter hormone levels in seeds efficiently and control seed dormancy and germination.

A PGSS based on the ecdysone receptor (EcR) and methoxyfenozide (MOF) has been previously described. This EcR-based PGSS is an efficient, inducible system which uses an established, and environmental safe inducing agent, methoxyfenozide. This PGSS consists of three basic components:

1) A receptor fusion protein that responds to a suitable ligand to activate gene expression. The exemplary chimeric receptor protein VGE comprises the VP16 activation domain from herpes simplex virus (“V”, amino acids 413-490) (18); the DNA binding domain of yeast GAL4 protein (“G”, amino acids 1-147) (19); and the ecdysone binding region from the ecdysone receptor (EcR) from the spruce budworm Cloristoneura fumiferana (“E”, amino acids 206-539) (20); Activation of this construct by the addition of methoxyfenozide induces the ligand dependent dimerization of the encoded fusion protein which leads to the expression of construct (2) below.

An inducible promoter element that when bound with the receptor fusion protein (1), activates expression of a target gene linked with the inducible promoter; and

3) A small molecule inducer (e.g. methoxyfenozide, MOF), which can bind to and activate the EcR (15, 16). In this study, potential applications of PGSS to modifying hormonal levels in seeds and their performance in terms of seed dormancy and germination were examined by modifying the expression of enzymes involved in seed germination.

To test if induction of NCED6, a gene encoding the rate-limiting ABA biosynthesis enzyme (FIG. 6) suppresses seed germination, the coding region of NCED6 was cloned in an EcR-based PGSS vector, similar to that detailed above.

The vector contained both the switch construct to make a chimeric receptor protein and the inducible promoter responsive to the molecular switch (FIG. 1). This next generation gene switch construct replaces the VP16 activation domain from herpes simplex virus (“V”, amino acids 413-490) (21) with the an activation domain from rice bZIP protein RF2a (“A”) (23) (Shown schematically in FIG. 1). This construct was used for the experiments below. This vector has the advantage of lacking any components derived human viruses, and is preferred for applications to food crops and environmental release.

### Vector Construction and Plant Transformation

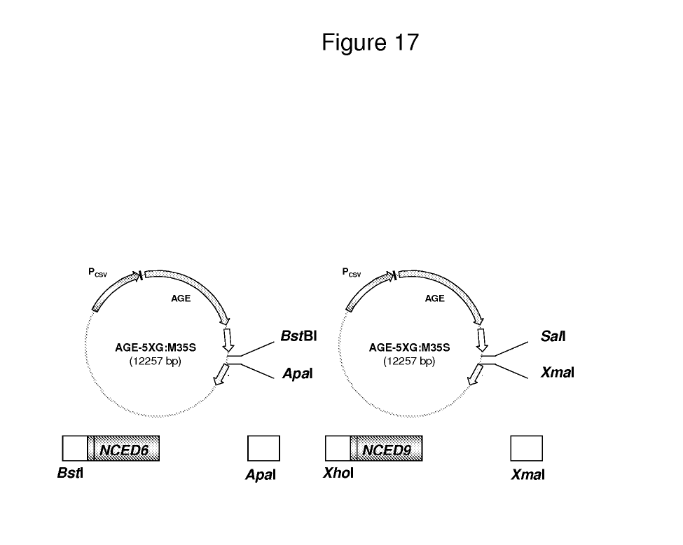

Vector construction and transformation were performed as follows: The NCED6 and NCED9 DNA were amplified from the Arabidopsis Columbia-0 genomic DNA (no introns in both genes) using gene specific primers (NCED6 forward 5′-CATAGGTCGCTCACAAGTCA-3′ (SEQ. ID. No. 32) and reverse 5′-ACGAAGAGAGTGTTGCATGGT-3′(SEQ. ID. No. 33); NCED9 forward: 5′-CGAATGTCTCACATCGTTGGT-3′ (SEQ. ID. No. 34) and reverse: 5′-AGGTCTCGAAGAGGAAGATG-3′(SEQ. ID. No. 35)) primers and a high fidelity DNA polymerase PrimeSTAR (Takara). Using amplified fragments as templates, the coding regions were amplified with restriction enzyme sites (BstBl/Apal for NCED6; Xhol/Xmal for NCED9). The conditions for PCR were: one cycle at 94° C. (4 min), one cycle at 80° C. (2 min), touchdown cycles (94° C. for 15 sec, 72° C.→66° C. for 15 sec, and 72° C. for 30 sec) (one cycle for each temperature) and 30 cycles at 94° C. (15 sec), 65° C. (15 sec) and 72° C. (30 sec), followed by extension at 72° C. (7 min). The NCED6 and NCED9 coding regions of SEQ. ID. No. 1 and SEQ. ID. No. 2, respectively, with restriction sites were cloned to Zero Blunt TOPO vectors and verified by sequencing. The coding regions were cut out with the restriction enzymes mentioned above and ligated to the corresponding restriction sites in the AGE gene switch vector (22) (FIG. 17), which contained an activation domain from rice bZIP protein RF2a (23)(“A”); the DNA binding domain of yeast GAL4 protein (“G”, amino acids 1-147) (19); and the ecdysone binding region from the EcR receptor from the spruce budworm Cloristoneura fumiferana (“E”, amino acids 206-539) (20). The sequences in the transformation vectors were verified again (termed AGE:NCED6 and AGE:NCED9 The transformation vectors were introduced into Agrobacterium tumefaciens strain GV3101 by electroporation, which were used to transform Arabidopsis thaliana Columbia-0 by the floral dip method (41).

### Example 1

### Transformation and Testing of the PGSS Vector

Wild-type Arabidopsis Columbia-0 plants were transformed with Agrobaterium harboring the AGE vector that contained the coding region of NCED6 downstream of the inducible promoter, as described above (called AGE:NCED6 hereafter). Twenty AGE:NCED6 transgenic lines were recovered, which did not show any specific phenotypes distinguishable from wild-type plants. Homozygous lines were isolated from five independent transgenic lines exhibiting 3:1 ratio segregation in antibiotic resistance and were used for further characterization.

### Example 2

### ABA Biosynthesis Gene Expression from PGSS Vector

Expression of a rate-limiting ABA biosynthesis gene was induced in the Arabidopsis plants produced in Example 1. The induction of NCED6 in the transgenic plants were tested by drenching the ligand Intrepid®2F solution, containing MOF as an active ingredient, to the Arabidopsis plants at approximately 10-rosette-leaf stages.

Briefly, for the induction of AGE:NCED6 plants at ˜10 rosette stages, diluted (×10,000), Intrepid®2F (Dow AgroSciences) solution which contained 62 μM MOF as an active ingredient was applied directly to the soil in pots containing Arabidopsis seedlings by drenching. Seedling were harvested two days after induction and frozen at −80° C. before RNA extraction. For the induction experiments in imbibed seeds, seeds were placed in 9 cm plastic petri dishes on two layers of filter paper (No. 2, Whatman Inc) moistened with 3.5 mL water or Intrepid®2F (×10,000) and incubated at 4° C. for 3 days and at 22° C. for 29 h (gene expression analysis) or 5 days (germination tests). For germination recovery, 10 μM fluridone was included with Intrepid®2F.

For RNA gel blot analysis, total RNA was extracted from Arabidopsis seedlings, siliques or seeds using standard phenol-SDS extraction (43). Equal amounts (2 μg) of total RNA were separated on a 1.3% (w/v) agarose gel containing 7% (v/v) formaldehyde, transferred to a positively charged nylon membrane (Hybond-N+, Amersham Biosciences), and UV-cross linked. To make the RNA probe, NCED6 cDNA in TOPO pCR 4.0 vector (Invitrogen) was transcribed using a digoxigenin-labeled NTP mixture (Roche Applied Science) and T7 RNA polymerase (Ambion). Overnight hybridization was done at 60° C. in hybridization buffer containing 50% (v/v) deionized formamide, 4% (w/v) blocking reagent (Roche Applied Science), 0.2% (w/v) SDS, 5×SSC, and approximately 100 ng mL⁻¹ RNA probe followed 15 min of prehybridization at the same temperature. The membranes were washed once for 25 min with 2×SSC and 0.1% (w/v) SDS at 60° C. and twice for 25 min with 0.2×SSC and 0.1% (w/v) SDS at 60° C. They were then blocked for 30 min with 5% (w/v) nonfat milk in 0.1 M maleic acid buffer, pH 7.5, containing 0.15 m NaCl and 0.3% (v/v) Tween 20 (buffer A) and were incubated with alkaline phosphate-conjugated anti-digoxigenin antibody (1:15,000 dilution) for 1 h at 25° C. After washing with buffer A, the membranes were subjected to chemiluminescence detection. The signal was detected on x-ray film.

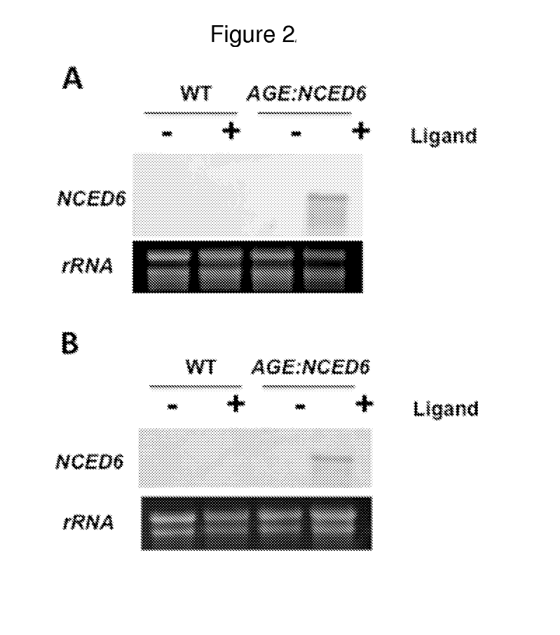

Ligand application caused the induction of NCED6 in the transgenic plants (FIG. 2A) NCED6 transcripts were not detectable in the AGE:NCED6 plants in the absence of the ligand, or wild-type plants both in the presence and absence of the ligand. These results validated that the AGE:NCED6 constructs were functional in the transgenic plants. In the induction experiments, gene expression was examined two days after drenching. Surprisingly, responses of the AGE:NCED6 plants to the ligand are very rapid. NCED6 is a seed-specific NCED. Analysis confirmed little or no expression of NCED6 in wild-type Arabidopsis rosettes (FIG. 2A). A potential problem with inducible gene expression systems is leaky expression of a target gene in the absence of a ligand. In the PGSS vector, AGE was driven by the Cassaya vein mosaic virus (CsVM) promoter, a strong constitutive promoter (FIG. 1). However, NCED6 expression was not detected in the transgenic plants in the absence of the ligand (FIG. 2A), suggesting that basal leaky expression is negligible in this system. These results confirm that the EcR-based system provides a reliable technology for conditional gene expression.

To test the induction of NCED6 in seeds, wild-type and the AGE:NCED6 seeds were imbibed for 29 h in the presence or absence of Intrepid®2F and used for gene expression analysis. Specific induction of NCED6 was observed in the AGE:NCED6 seeds treated with the ligand (FIG. 2B) NCEDs including NCED6 are expressed in developing seeds and imbibed dormant seeds of wild type. We did not detect NCED6 in imbibed wild-type Col seeds, which were germinable (FIG. 2B). The AGE:NCED6 seeds imbibed in the absence of the ligand also showed no leaky expression.

### Example 3

### Germination Control

Germination was controlled by inducing expression of a rate-limiting ABA biosynthesis gene, as in Example 2.

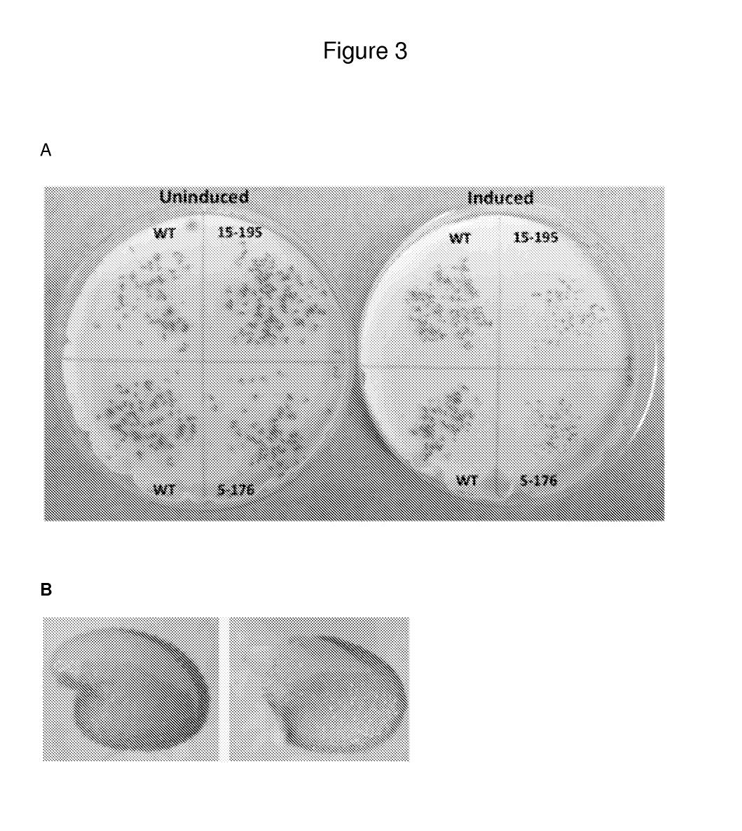

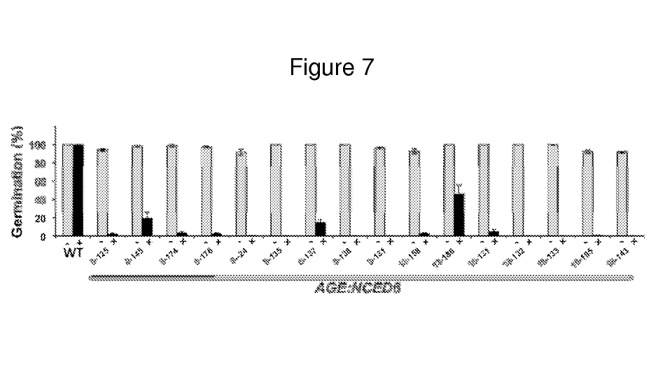

Surprisingly, actual suppression of seed germination was observed in the AGE:NCED6 seeds induced by the ligand, Intrepid®2F (FIG. 3A). The suppression was not observed in the AGE:NCED6 seeds in the absence of the ligand. Intrepid®2F did not affect germination of wild-type seeds. Specific induction of germination suppression by the ligand was observed in many individual seed lots from the five homozygous independent transgenic lines. Near complete suppression of radicle emergence and no seedling establishment were observed in many seed lots tested, except for a few lines showing moderate germination (FIG. 7) Arabidopsis seeds initially rupture testa (seed coat) and then endosperm rupture occurs when the radicle emerges under normal germination conditions (26). The majority (77-89%) of the induced AGE:NCED6 seeds that failed to complete germination were arrested after testa rupture (FIG. 3, left). The rest of arrested seeds exhibited endosperm rupture, however their protruded radicles did not continue to grow (FIG. 3B, right). These events mimic germination suppression by exogenous ABA. Arabidopsis (and other plant) testa is impermeable to some small substances. It is possible that Intrepid®2F does not enter seeds before testa rupture. In contrast, Arabidopsis endosperm seems to be permeable to the ligand, since many seeds were arrested right after testa rupture (FIG. 3B) and the induction of NCED6 expression by the ligand was detected at 29 h-imbibition when most seeds had completed testa rupture but no radicle protrusion occurred yet (FIG. 2B).

The suppression of germination by Arabidopsis NCED6 induction was surprisingly effective. All tested homozygous AGE:NCED6 lines exhibited clear inhibition of germination specifically in the presence of the ligand. The suppression of radicle elongation without or immediately after testa rupture indicates that the induction of NCED6 affected the growth potential of the embryo immediately after the entrance of the ligand into seed tissues, which confirms the robustness of the gene switch system and provides proof that NCED6 is the right target to manipulate to alter ABA in seeds. Moreover, leaky expression (germination inhibition in the absence of the ligand) was not observed at all.

### Example 4

### Additional or Alternative Germination Control Systems

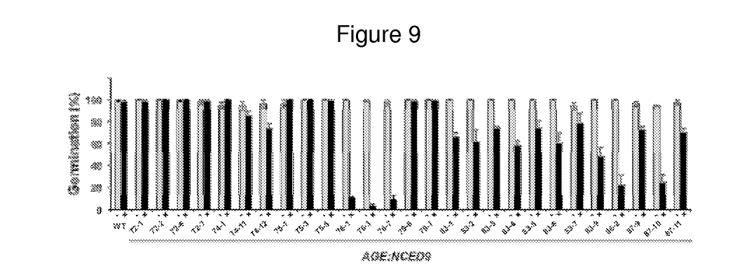

A further PGSS system was constructed, in which the NCED6 gene was replaced with NCED9 to create an AGE:NCED9 vector. The vector was transformed into Arabidopsis, and expression of NCED9 was induced by administration of the ligand, as above. Germination control was examined. The results are shown in FIG. 9. As depicted in FIG. 9, seed germination can be controlled by placing NCED9 under the control of a gene switch. In these experiments, manipulating NCED6 expression appears to be more efficient than manipulating NCED9 expression for modulating germination.

### Example 5

### Germination Control by Altered ABA Levels in Seeds

The inventors reasoned that the germination suppression induced by NCED6 expression was most likely caused by changes in ABA biosynthesis. To examine whether ABA levels is the AGE:NCED6 seeds were affected by gene induction, ABA in wild-type and the AGE:NCED6 seeds was quantified.

ABA was quantified by a previously published method (44), but was modified to ABA. Briefly, samples were ground in liquid nitrogen and internal standards (10 mL of 2.5 mM) were added. Samples were extracted with 1.5 mL acetonitrile/methanol (1:1 v:v). After lyophilization, samples were resolubilized in 200 mL of 50% MeOH. For LC separation, two monolithic C18 columns (Onyx, 4.6 mm×100 mm, Phenomenex) with a guard cartridge were used flowing at 1 mL min⁻¹. The gradient was from 40% solvent A (0.1% (v/v) acetic acid in MilliQ water), held for 2 min, to 100% solvent B (90% acetonitrile (v/v) with 0.1% acetic acid (v/v) in 5 min. The LC was held at 100% B for 3 min and then ramped back to initial conditions and re-equilibrated for an additional 2 min. To minimize variation from the autosampler, the sample loop was overfilled with 52 mL of sample and the sample storage temperature was set to 8° C. The LC-MS/MS system was composed of a Shimadzu LC system with LEAP CTC PAL autosampler coupled to an Applied Biosystems 4000 QTRAP mass spectrometer equipped with a Turbolon Spray (TIS) electrospray ion source. Source parameters were set to: CUR: 25, GAS1: 50, GS2: 50 (arbitrary unit), CAD: high, 1HE: on, TEM: 550° C., IS: −4500. Both quadruples (Q1 and Q3) were set to unit resolution. Analyst software (version 1.4.2) was used to control sample acquisition and data analysis. To maximize sensitivity, ABA standard solutions were infused into the 4000 QTRAP with a syringe pump (Harvard 22) at 10 mL min⁻¹ to select multiple reaction monitoring (MRM) transitions and optimize compound-dependent parameters for MRM detection. A standard curve was established for the method. For quantitation, a series of standards were prepared containing different concentrations of ABA mixed with D-labeled ABA (250 μmol/sample). Correction factors were obtained by adjusting the ratio of standard peak areas to that of internal standards in all samples. The peak areas of endogenous ABA were normalized with the corresponding internal standard and then calculated according to the standard curve.

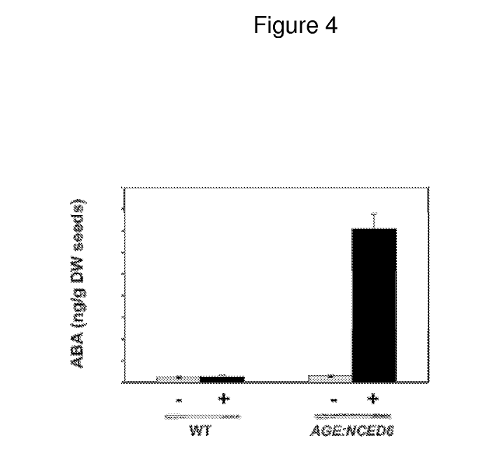

ABA levels in both wild-type and the AGE:NCED6 seeds in the absence of the ligand (Intrepid®2F) were relatively low and did not differ significantly. In contrast, the application of the ligand increased ABA drastically in the AGE:NCED6 seeds (FIG. 4). This result suggested that the induction of NCED6 expression actually altered the ABA biosynthesis pathway in seeds. Previous study indicated that the induction of gene expression by the VGE vector ranges from 50-fold in tobacco to up to 1000-fold in Arabidopsis. Surprisingly, induction of NCED6 by the AGE gene switch was high enough to cause drastic increase in ABA levels.

Seeds of cyp707a2 mutants that lack ABA 8′-hydroxylase, a key enzyme for ABA deactivation, exhibit hyperdormancy. The ABA levels in the induced AGE:NCED6 seeds were equivalent to those detected in the cyp707A2 hyperdormant mutant seeds (FIG. 4), which explains strong suppression of seed germination in the induced AGE:NCED6 seeds (FIG. 3; FIG. 7). In the case of the cyp707A2 mutant analysis, control wild type seeds under the experimental conditions contained higher levels of ABA compared to those in the uninduced AGE:N6 seeds and wild-type seeds in our experimental system. Therefore, the fold increase in ABA levels in the AGE:NCED6 induction is estimated higher (20 fold) than that caused by cyp707A2 mutation (6 fold).

### Example 6

### Germination Recovery by an ABA Biosynthesis Inhibitor

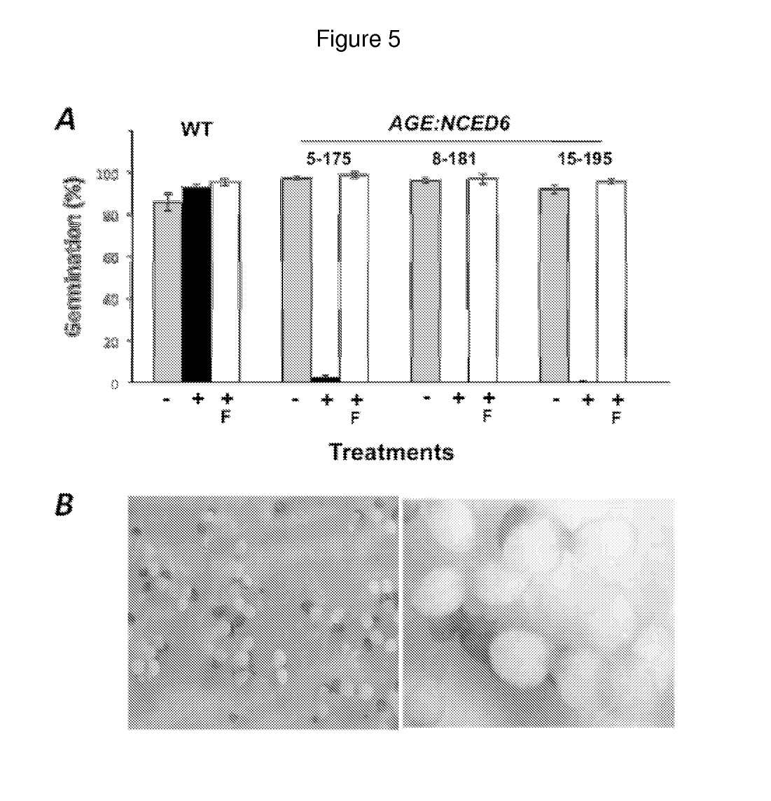

ABA quantification provided supporting evidence that germination suppression in the AGE:NCED6 seeds were due to the changes in ABA levels in seeds, as described above. To examine this further, the inventors tested germination of the AGE:NCED6 seeds in the presence of fluridone, a carotenoid biosynthesis inhibitor. Fluridone inhibits phytoene desaturase, a key enzyme in the carotenoid biosynthetic pathway, which is the upstream of ABA biosynthesis pathway (FIG. 6A). Even when seeds are able to over-produce functional NCED enzyme proteins, if the substrates for the enzyme are not supplied through the carotenoid biosynthesis pathway, ABA levels cannot be increased by the over-induction of NCED6. The inventors examined germination of the AGE:NCED6 seeds with co-application of the ligand (Intrepid®2F) and fluridone. Germination of the AGE:NCED6 seeds that was suppressed by the ligand was fully rescued by fluridone (FIG. 5A). The induced AGE:NCED6 seeds were able to germinate and develop seedlings, although they were etiolated due to the herbicidal effects of the chemical (FIG. 5B). These results further indicate specific suppression of germination induced in the AGE:NCED6 seeds was indeed dependent on ABA biosynthesis.

### Example 7

### Suppression of Germination in Non-Dormant Mutant Seeds

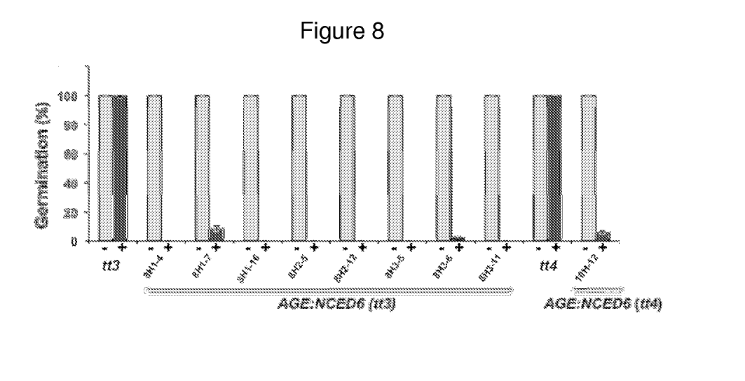

Transgenic lines expressing AGE:NCED6 in transparent testa (tt) mutant background. tt mutants lack pigments in testa (28, 33). An AGE:NCED6 vector was transformed into tt3 and tt4. TT3 and TT4 encode dihydroflavonol 4-reductase (DFR) and chalcone synthase (CHS), respectively, which are involved in proanthocyanidin (PA) biosynthesis (34, 35). PA plays an essential role in imposing seed dormancy, therefore tt mutant seeds exhibit little or no dormancy. A surplus of transgenic plants were not recovered for the tt lines due to the hypersensitivity of tt seeds to sterilization and antibiotics used in screening. However, the isolated lines showed clear responses to the induction by the ligand. The AGE:NCED6 seeds in both tt3 and tt4 background exhibited suppression of germination specifically in the presence of Intrepid®2F (FIG. 8). Surprisingly, these results demonstrated that germination of even extreme non-dormant seeds can be suppressed by altering the ABA biosynthesis genes. Examples of such extreme non-dormant seeds (e.g. used in agriculture) include wheat grains which are susceptible to germination during seed development on the maternal plant. This is called pre-harvest sprouting or PHS, a serious agricultural problem causing significant economical losses.

### Example 8

### Gene Switch Technology for Seed Development, Dormancy and Germination in Camelina

The experiments using the ABA biosynthesis genes as a model in the present work demonstrated that the gene switch technology can be used to alter hormone levels in seeds and directly impact seed dormancy and germination. The results of germination recovery in the AGE:NCED6 seeds by fluridone especially provided an important implication that the substrates for NCEDs are continuously supplied even in non-dormant seeds during imbibition. In other words, seeds have potentials to express different phenotypes responding to the gene switch containing the rate-limiting genes for hormone biosynthesis and deactivation.

PGSS using the seed-specific NCED6 enabled complete and near-complete suppression of germination in imbibed seeds. NCED6 is specifically expressed in the endosperm of developing seeds while NCED9 is expressed in the endosperm and the peripheral regions of the embryo during seed development.

Without being bound to any one particular theory of operation is possible that the difference observed between the AGE:NCED6 and AGE:NCED9 seeds, in terms of germination suppression, reflects the nature of two genes, or differences in enzyme catalytic activity. Alternatively, since exogenously applied ligand initially goes through the endosperm and then reaches the embryo, the endosperm-specific NCED6 might be more suitable for the first site of induction (endosperm). In any case, based on this knowledge, the experiments suggest that the manipulation of NCED6 is surprisingly better for regulating seed germination compared to NCED9.

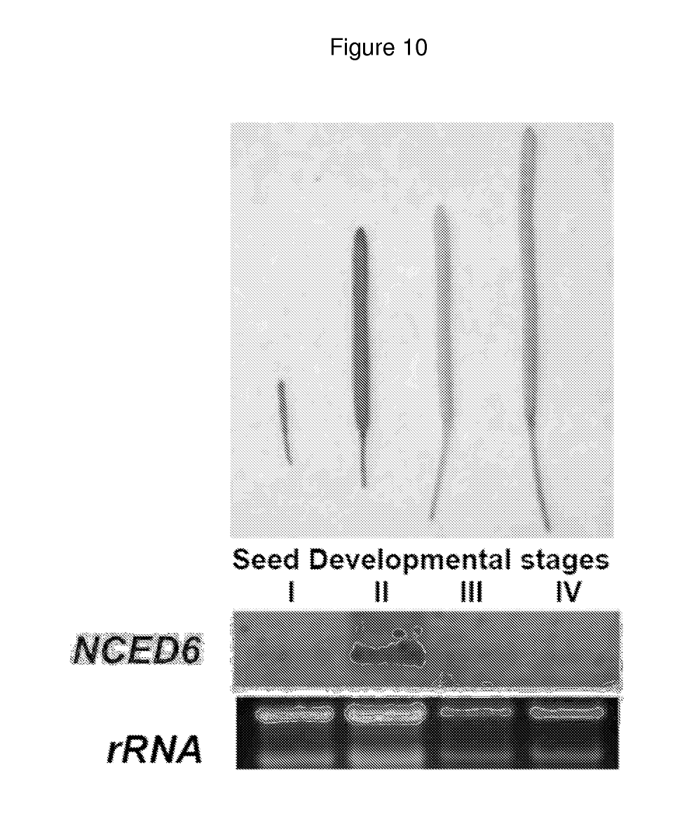

The NCED6 promoter can optionally be used to drive trans-acting factor for tissue-specific gene switch induction, and peak expression of NCED6 is detected around long-green-silique stages (FIG. 10), suggesting that it might represent a good potential candidate to drive NCED6 to regulate seed germination.

Since NCED6 induction exhibited near-complete inhibition of germination during imbibition, at least NCED6 induction during seed development by the gene switch is expected to increase ABA levels and cause hyperdormancy. Under these conditions simple drenching would supply the ligand to many organs, but NCED6 expression would be increased specifically in the right tissues in developing seeds at the right timing.

Alternatively, spontaneous hyperdormancy can be created by driving NCED expression with strong seed-specific promoters that are activated at the stages, during which the native NCEDs are expressed. Under these circumstances seed germination could be recovered via the induction of an ABA deactivation gene such as CYP707A2.

To test the hypothesis that the combination of the negative and positive regulators with spontaneous or inducible over-expression strategies will provide a comprehensive system to prevent unwanted seed germination, the following expression constructs were prepared and tested in Camelina.

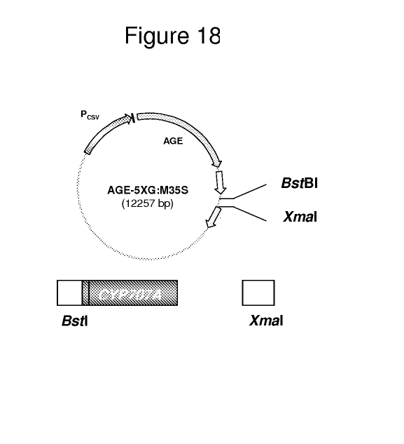

The AGE gene switch vector, as described above comprising; an activation domain from rice bZIP protein RF2a (“A”); the DNA binding domain of yeast GAL4 protein (“G”); and the ecdysone binding region from the EcR receptor (“E”) and a DNA (5XUAS) element operatively coupled to a minimal promoter that binds to the GAL4 DNA binding domain, operatively coupled to a nucleic acid encoding ABA 8′ hydroxylase (FIG. 18) (SEQ. ID. No. 3).

3) A constitutive hormone metabolism gene cassette comprising; an endogenous NCED6 promoter (SEQ. ID. No 36) operatively coupled to the NCED6 coding region (SEQ. ID. No. 1) and NCED6 nos 3′ terminator (SEQ. ID. No. 37)

General Methods

For the induction experiment in imbibed seeds, seeds were placed in 9 cm plastic petri dishes on two layers of filter paper (No. 2, Whatman Inc.) moistened with 4 mL water or (122.6 μM or 2×10,000) MOF or 1 mM ABA and incubated at 4° C. for overnight and at 22° C. for 72 h (germination tests and gene expression analysis). For germination recovery, 10 M fluridone was included.

Results

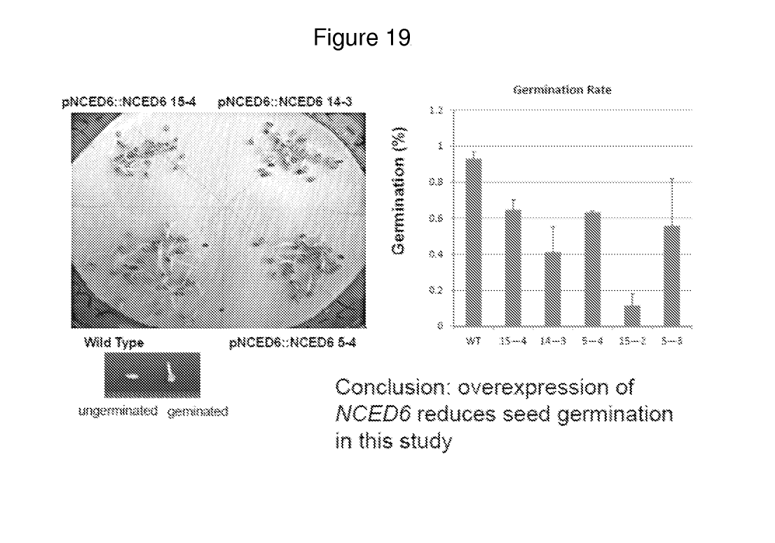

To test whether over expression of Arabidopsis NCED6 in Camelina, when expressed from the endogenous NCED6 promoter, results in an ABA-related effect during seed germination, a construct carrying Arabidopsis NCED6 driven by the Arabidopsis NCED6 gene promoter was made as described above (pNCED6:NCED6) and transformed into Camelina wild type plants, as described previously. T3 progeny seeds of several independent homozygous lines were selected as the final study targets, clonal cell lines 14-3, 5-3, and 15-2 were identified based on a segregation with a 3:1 ratio for kanamycin resistance in T2 seeds. Germination test by just using water revealed that over expression of these NCED6 three independent transgenic lines, 14-3, 5-3, and 15-2, reduced seed germination during imbibitions. (FIG. 19)

To test whether over expression of Arabidopsis NCED6 in Camelina when expressed from the AGE inducible expression system, resulted in an inhibition of seed germination, wild-type Camelina plants were transformed with Agrobacterium harboring the AGE: NCED6 described previously. Twenty AGE: NCED6 transgenic lines were recovered, none of which displayed phenotypes distinguishable from wild-type plants. Homozygous lines were isolated from 3 independent transgenic lines that exhibited a 3:1 ratio of segregation of the antibiotic resistance trait, and were used for further studies.

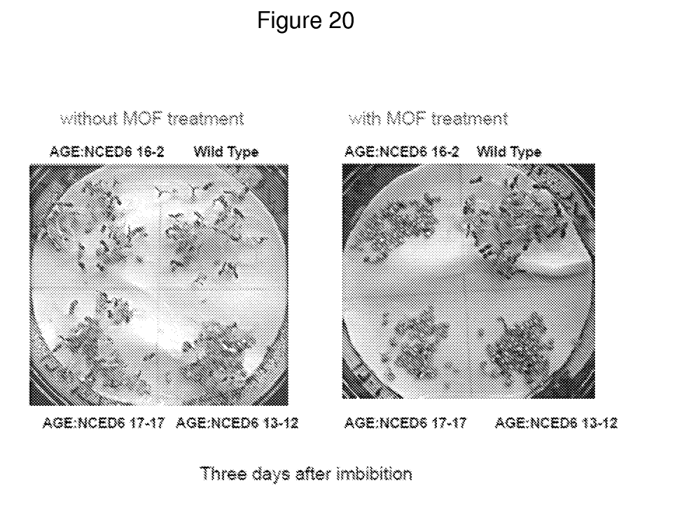

To determine whether there was an effect of the AGE: NCED6 expression vector on the ability of transgenic seeds to undergo germination, wild-type and homozygous seeds from transgenic plant lines, 16-12, 17-17, and 13-12, were imbibed for three days in the presence or absence of MOF. Total RNA was extracted and used for real time PCR analysis but we did not detect Arabidopsis NCED6 expression, probably it did not accumulate detectible amounts of Arabidopsis NCED6-specific mRNAs. However, studies on examining seed germination in AGE: NCED6 seeds and wild type Camelina seeds showed that transgenic seeds were inhibited dramatically in the presence of MOF (FIG. 20). Thus, the major suppression of radicle emergence and absence of seedling establishment were observed in many seed lots tested, except for a few lines which showed moderate germination, but this did not occur in the absence of MOF. Control seeds of non-transgenic plants were shown the same in the presence or absence of MOF. Germination of Camelina seeds is observed initially as ruptured testa followed by emergence of the endosperm when the radicle emerges. In our study, three AGE:NCED6 transgenic lines, 16-12, 17-17, and 13-12, failed to exhibit endosperm rupture and protrude radicles to complete germination by the inhibition of 89%, 90%, and 95% among, respectively.

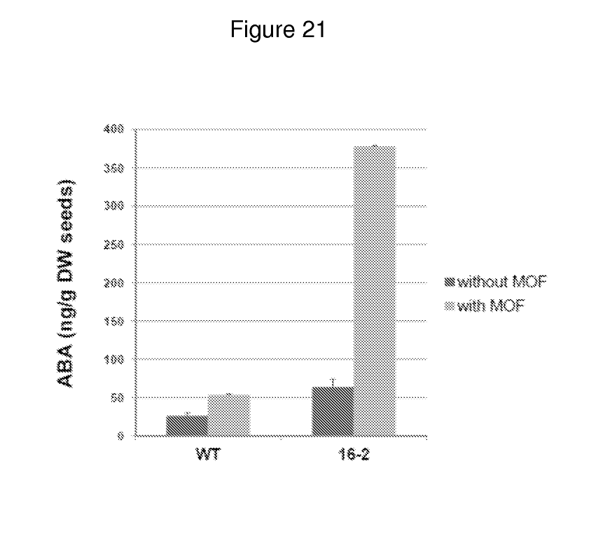

To examine whether ABA levels in the AGE: NCED6 seeds were affected by gene induction, ABA in wild-type and the AGE: NCED6 seeds was quantified by mass spectrometry, as described previously. In the absence of MOF, ABA levels in both wild-type and the AGE: NCED6 seeds were relatively low and similar to each other. However, followed by application of the MOF, there was a remarkable increase in ABA in AGE: NCED6 seeds (FIG. 21). This result suggested that the induction of NCED6 expression might alter the ABA biosynthesis pathway in seeds.

To confirm whether the inhibition of germination in plants transformed with the AGE: NCED6 expression vector, was caused by the production of ABA, seeds were germinated in the presence of fluridone, a carotenoid biosynthesis inhibitor. Fluridone inhibits phytoene desaturase, a key enzyme in the carotenoid biosynthetic pathway, upstream of the role of NCED6 in ABA biosynthesis. It is known that seed dormancy can be released by fluridone by blocking the upstream events. Seeds containing the AGE: NCED6 gene were germinated with co-application of MOF and fluridone. Germination of the AGE:NCED6 seeds was fully rescued by fluridone (data not shown) and seeds that were induced germinated and developed to seedlings, although they were etiolated due to the herbicidal effects of the chemical. These results support the conclusion that specific suppression of germination in seeds of AGE: NCED6 plants induced with MOF was dependent on ABA biosynthesis.

To test whether the induction of CYP707A2 gene, a gene encoding ABA 8′-hydroxylases from Arabidopsis, suppresses seed dormancy in Camelina under the control of PGSG in which the CYP707A2 gene replaces the coding region of NCED6 in the AGE: NCED6 expression vector, a new vector named as AGE: CYP707A2 was constructed. Similarly, wild-type Camelina plants were transformed with Agrobacterium harboring the AGE: CYP707A2. Nineteen AGE: CYP707A2 transgenic lines were recovered, none of which displayed phenotypes distinguishable from wild-type plants. Homozygous lines were isolated from 3 independent transgenic lines that exhibited a 3:1 ratio of segregation of the antibiotic resistance trait, and were used for further studies.

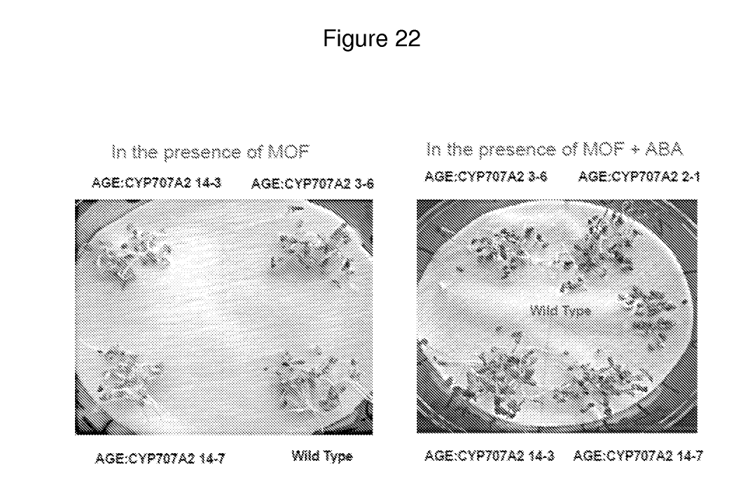

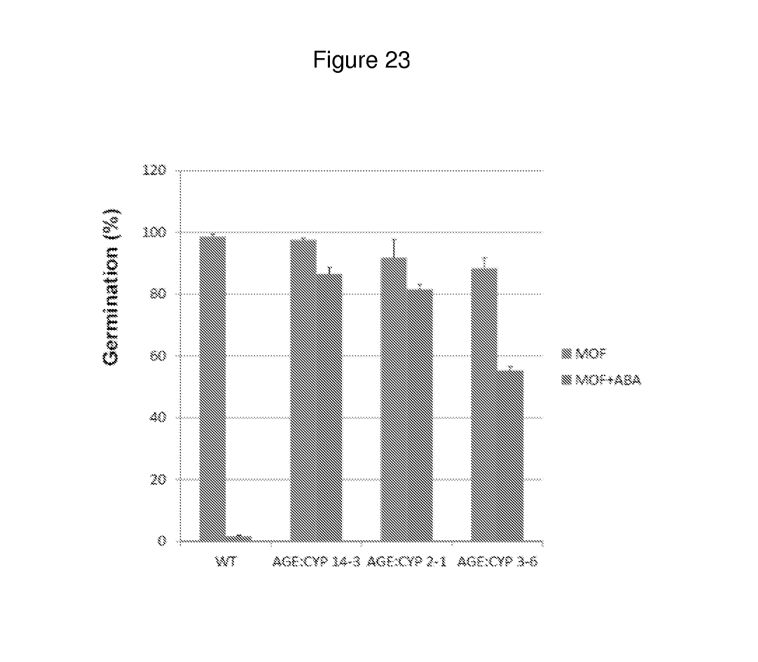

To determine whether there was an effect of AGE: CYP707A2 transgenic seeds on germination during imbibitions, wild-type and homozygous seeds from transgenic plant lines, 14-3, 3-6, and 2-1, were imbibed for three days in the presence of MOF or in the presence of MOF+ABA. Studies showed (FIG. 22) that transgenic seeds were grown dramatically in the presence of MOF or MOF+ABA by 99%, 98%, and 95%. But control seeds of non-transgenic plants were shown the near-complete inhibition in the presence of MOF+ABA. (FIG. 23) Induced promotion of germination by MOF was observed in at least three independent transgenic lines, in three biological replicates.

### Example 9

### Suppression of Precocious Germination

Transgenic plants expressing NCED6 were produced, as described above. Precocious germination tests which mimicked preharvest sprouting in fields

Briefly, developing siliques at the long-green stage (FIG. 10) were collected, slightly opened at the replum-valve margin using a surgical blade, sterilized with 70% (v/v) ethanol for 1 min and 25% bleach for 10 min, and then plated on 0.7% (w/v) agar containing 1% (w/v) sucrose and MS salt (44), with or without Intrepid®2F (×10,000). For three independent homozygous AGE:NCED6 lines and wild type, 10 siliques from each of three individual plants were divided into two groups of five, which were plated in the presence or absence of the ligand, respectively. Germination was examined after 12 days of incubation.

Surprisingly, precocious germination was inhibited to quite a remarkable extent in transgenic lines expressing the seed-dormancy gene.

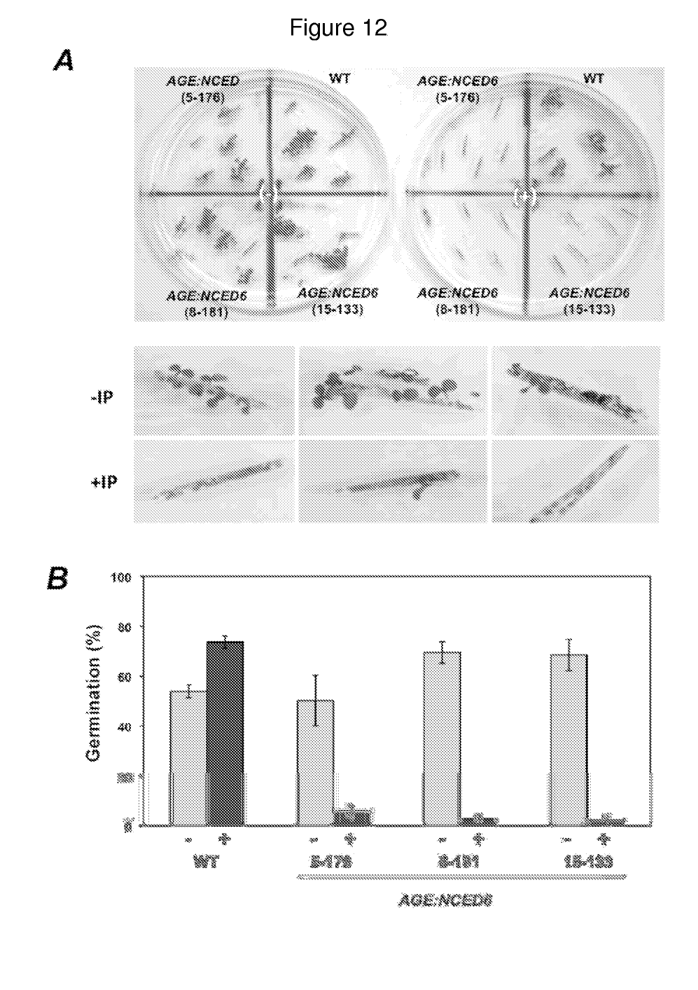

Germination from the developing seeds contained in young green siliques of the AGE:NCED6 lines was suppressed effectively by the ligand application (FIG. 12).

Specifically, FIG. 12 depicts: (A) Photographs showing immature green siliques incubated for 12 d on agar media. Upper panel shows siliques of wild-type (WT) and the AGE:NCED6 lines (5-176, 8-181 and 15-133) incubated in the absence (−) or presence (+) of the ligand. Note that the induced AGE:NCED6 siliques exhibit little germination. Bottom panels show representative images of precocious germination in the absence of the ligand (−IP) and the suppression of precocious germination in the presence of the ligand (+IP) in the AGE:NCED6 siliques. (B) Results of precocious germination tests of wild-type (WT) and the AGE:NCED6 siliques in the absence (−) or presence (+) of the ligand. Each data indicates average; SD (n=3).

While there are multiple inducible gene expression systems which have been used successfully in experiments to modify seed germination (40), many of them use ligands that may not be easily adapted to application in agricultural practices, such as steroid hormones or antibiotics. The vectors used for those systems also contain some components from human virus, such as the VP16 activation domain from herpes simplex virus (21).

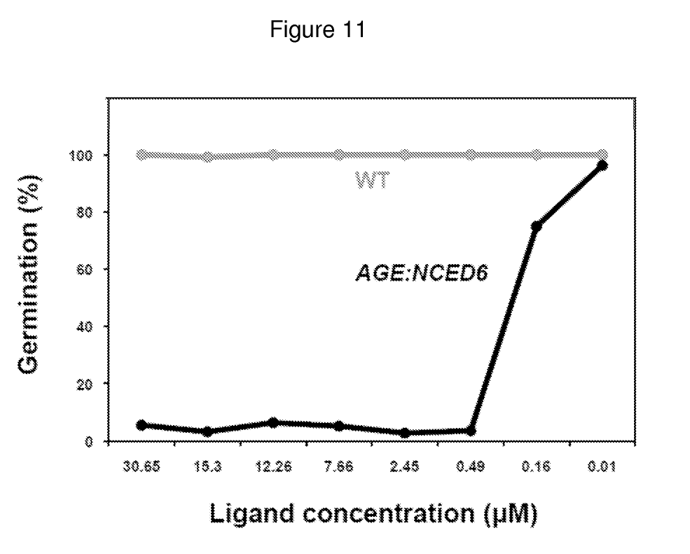

By contrast the present study demonstrated that the AGE system in which the VP16 activation was replaced by the plant-origin activation domain RF2a, functioned properly in seeds. Although we used ×10,000 dilution of the ligand, Intrepid®2F (˜62 μM MOF), throughout the experiments, dose response experiments using the AGE:NCED6 seeds have indicated that the ligand can still be diluted further ×100 without losing its effects to suppress seed germination (FIG. 11). The ligand has been approved by U.S. Environmental Protection Agency. Therefore, the technology is highly applicable to agriculture. This newly developed system with the plant-origin activation domain will move one step forward in the efforts to create a “green” PGSS that is suitable for application to seed production and treatment.

The citations provided herein are hereby incorporated by reference for the cited subject matter.

### REFERENCES**

1. Huang G & Varriano-Marston E (1980) alpha-Amylase activity and preharvest sprouting damage in Kansas hard white wheat. Journal of Agricultural and Food Chemistry 28(3):509-512.
2. Yamaguchi S, Kamiya Y, & Nambara E (2007) Regulation of ABA and GA levels during seed development and germination in Arabidopsis (Blackwell Publishing, Oxford) pp 224-247.
3. Lefebvre V, et al. (2006) Functional analysis of Arabidopsis NCED6 and NCED9 genes indicates that ABA synthesized in the endosperm is involved in the induction of seed dormancy. The Plant Journal 45(3):309-319.
4. Nambara E & Marion-Poll A (2005) ABSCISIC ACID BIOSYNTHESIS AND CATABOLISM. Annual Review of Plant Biology 56(1):165-185.
5. Tan B C, Schwartz S H, Zeevaart J A D, & McCarty D R (1997) Genetic control of abscisic acid biosynthesis in maize. Proceedings of the National Academy of Sciences 94(22):12235-12240.
6. Burbidge A, et al. (1999) Characterization of the ABA-deficient tomato mutant notabilis and its relationship with maize Vp14. The Plant Journal 17(4):427-431.
7. Destefano-Beltrán L, Knauber D, Huckle L, & Suttle J (2006) Effects of postharvest storage and dormancy status on ABA content, Mabolism, and expression of genes involved in ABA biosynthesis and Mabolism in potato tuber tissues. Plant Molecular Biology 61(4):687-697.
8. Chemys J T & Zeevaart J A D (2000) Characterization of the 9-Cis-Epoxycarotenoid Dioxygenase Gene Family and the Regulation of Abscisic Acid Biosynthesis in Avocado. Plant Physiol. 124(1):343-354.
9. Rodrigo M-J, Alquezar B, & Zacarias L (2006) Cloning and characterization of two 9-cis-epoxycarotenoid dioxygenase genes, differentially regulated during fruit maturation and under stress conditions, from orange (Citrus sinensis L. Osbeck). J. Exp. Bot. 57(3):633-643.
10. Iuchi S, et al. (2001) Regulation of drought tolerance by gene manipulation of 9-cis-epoxycarotenoid dioxygenase, a key enzyme in abscisic acid biosynthesis in Arabidopsis. The Plant Journal 27(4):325-333.
11. Kushiro T, et al. (2004) The Arabidopsis cytochrome P450 CYP707A encodes ABA 8′-hydroxylases: key enzymes in ABA catabolism. EMBO J. 23(7):1647-1656.
12. Crozier A, Kamiya Y, Bishop G, & Yokota T (2000) Biosynthesis of hormones and elicitor molecules (American Society of Plant Biologists, Rockville) pp 850-929.
13. Yamaguchi S, Smith M W, Brown R G S, Kamiya Y, & Sun T-p (1998) Phytochrome regulation and differential expression of gibberellin 3β-hydroxylase genes in germinating Arabidopsis seeds. Plant Cell 10(12):2115-2126.
14. Mitchum M G, et al. (2006) Distinct and overlapping roles of two gibberellin 3-oxidases in Arabidopsis development. The Plant Journal 45(5):804-818.
15. Padidam M (2003) Chemically regulated gene expression in plants. Current Opinion in Plant Biology 6(2):169-177.
16. Koo J C, Asurmendi S, Bick J, Woodford-Thomas T, & Beachy R N (2004) Ecdysone agonist-inducible expression of a coat protein gene from tobacco mosaic virus confers viral resistance in transgenic Arabidopsis. The Plant Journal 37:439-448.
17. Tavva V, Dinkins R, Palli S, & Collins G (2007) Development of a tightly regulated and highly inducible ecdysone receptor gene switch for plants through the use of retinoid X receptor chimeras. Transgenic Research 16(5):599-612.
18. Dalrymple M A, McGeoch D J, Davison A J, & Preston C M (1985) DNA sequence of the herpes simplex virus type 1 gene whose product is responsible for transcriptional activation of immediate early promoters. Nucl. Acids Res. 13(21):7865-7879.
19. Laughon A & Gesteland R F (1984) Primary structure of the Saccharomyces cerevisiae GAL4 gene. Mol. Cell. Biol. 4(2):260-267.
20. Perera S C, et al. (1999) An analysis of ecdysone receptor domains required for heterodimerization with ultraspiracle. Archives of Insect Biochemistry and Physiology 41(2):61-70.
21. Dalrymple M A, McGeoch D J, Davison A J, & Preston C (1985) DNA sequence of the herpes simplex virus type 1 gene whose product is responsible for transcriptional activation of immediate early promoters. Nucl. Acids Res. 13(21):7865-7879.
22. Ordiz M I, Yang J, Barbazuk W B, & Beachy R N (Functional analysis of the activation domain of RF2a, a rice transcription factor. Plant Biotechnology Journal 9999(9999).
23. Dai S, et al. (2003) Functional analysis of RF2a, a rice transcription factor. (Translated from eng) J Biol Chem 278(38):36396-36402 (in eng).
24. Padidam M, Gore M, Lily Lu D, & Smirnova O (2003) Chemical-inducible, ecdysone receptor-based gene expression system for plants. Transgenic Research 12(1):101-109.
25. Finch-Savage W E, Cadman C S C, Toorop P E, Lynn J R, & Hilhorst H W M (2007) Seed dormancy release in Arabidopsis Cvi by dry after-ripening, low temperature, nitrate and light shows common quantitative patterns of gene expression directed by environmentally specific sensing. The Plant Journal 51(1):60-78.
26. Liu P P, et al. (2005a) Large-scale screening of Arabidopsis enhancer-trap lines for seed germination-associated genes. Plant J 41(6):936-944.
27. Muller K, Tintelnot S, & Leubner-Mzger G (2006) Endosperm-limited Brassicaceae seed germination: Abscisic acid inhibits embryo-induced endosperm weakening of Lepidium sativum (cress) and endosperm rupture of cress and Arabidopsis thaliana. Plant Cell Physiol. 47(7):864-877.
28. Debeaujon I, Leon-Kloosterziel K M, & Koornneef M (2000) Influence of the testa on seed dormancy, germination, and longevity in Arabidopsis. Plant Physiol 122(2):403-414.
29. Rajjou L, et al. (2004) The effect of alpha-amanitin on the Arabidopsis seed proteome highlights the distinct roles of stored and neosynthesized mRNAs during germination. Plant Physiol 134(4):1598-1613.
30. Bartels P G & Watson C W (1978) Inhibition of carotenoid synthesis by fluridone and norflurazon. Weed Science 26:198-203.
31. Chamovitz D, Sandmann G, & Hirschberg J (1993) Molecular and biochemical characterization of herbicide-resistant mutants of cyanobacteria reveals that phytoene desaturation is a rate-limiting step in carotenoid biosynthesis. Journal of Biological. Chemistry 268:17348-17353.
32. Grappin P, Bouinot D, Sotta B, Miginiac E, & Jullien M (2000) Control of seed dormancy in Nicotiana plumbaginifolia: post-imbibition abscisic acid synthesis imposes dormancy maintenance. Planta 210(2):279-285.
33. Debeaujon I & Koornneef M (2000) Gibberellin Requirement for Arabidopsis Seed Germination Is Determined Both by Testa Characteristics and Embryonic Abscisic Acid. Plant Physiol. 122(2):415-424.
34. Shirley B W, Hanley S, & Goodman H M (1992) Effects of ionizing radiation on a plant genome: Analysis of two Arabidopsis transparent testa mutations. Plant Cell 4(3):333-347.
35. Feinbaum R L & Ausubel F M (1988) Transcriptional regulation of the Arabidopsis thaliana chalcone synthase gene. Mol. Cell. Biol. 8(5):1985-1992.
36. Debeaujon I, Lepiniec L, Pourcel L, & Routaboul J M eds (2007) Seed coat development and dormancy (Blackwell Publishing, Oxford), pp 25-49.
37. Thompson A J, et al. (2000) Ectopic expression of a tomato 9-cis-epoxycarotenoid dioxygenase gene causes over-production of abscisic acid. The Plant Journal 23(3):363-374.
38. Fan J, Hill L, Crooks C, Doerner P, & Lamb C (2009) Abscisic acid has a key role in modulating diverse plant-pathogen interactions. Plant Physiol. 150(4):1750-1761.
39. Qin X & Zeevaart J A D (2002) Overexpression of a 9-cis-Epoxycarotenoid Dioxygenase Gene in Nicotiana plumbaginifolia Increases Abscisic Acid and Phaseic Acid Levels and Enhances Drought Tolerance. Plant Physiol. 128(2):544-551.
40. Piskurewicz U, et al. (2008) The gibberellic acid signaling repressor RGL2 inhibits Arabidopsis seed germination by stimulating abscisic acid synthesis and ABI5 activity. Plant Cell 20(10):2729-2745.
41. Clough S J & Bent A F (1998) Floral dip: a simplified Method for Agrobacterium-mediated transformation of Arabidopsis thaliana. Plant J 16(6):735-743.
42. Murashige T & Skoog F (1962) A revised medium for rapid growth and bioassays with tobacco tissue cultures. Physiologia Plantarum 15:473-497.
43. Sambrook J, Fritsch E F, & Maniatis T (1989) Molecular cloning: A Laboratory Manual (2nd edition) (Cold Spring Harbor Laboratory Press, Cold Spring Harbor, N.Y.).
44. Chen Q, Zhang B, Hicks L M, Wang S, & Jez J M (2009) A liquid chromatography-tandem mass spectroMry-based assay for indole-3-acetic acid-amido synthetase. Analytical Biochemistry 390(2):149-154.
45. Padidam M, Gore M, Lily Lu D, & Smirnova 0 (2003) Chemical-Inducible, Ecdysone Receptor-Based Gene Expression System for Plants. Transgenic Research 12:101-109.

1-102. (canceled)

103. A transgenic plant exhibiting controllable seed germination, comprising within its genome, and expressing, a heterologous polynucleotide encoding a 9-cis-epoxycarotenoid dioxygenase (NCED) and a polynucleotide encoding a trans-acting factor, wherein the polynucleotide encoding the 9-cis-epoxycarotenoid dioxygenase (NCED) is operably linked for expression to a promoter comprising a cis-element, wherein in the activity of the cis-element is regulated by the trans-acting factor, wherein the activity of the trans-acting factor with respect to the cis-element is regulated by a positive chemical modulator.

104. The transgenic plant of claim 103, wherein the 9-cis-epoxycarotenoid dioxygenase (NCED) is selected from among a NCED1, a NCED2, a NCED3, a NCED4, a NCED5, a NCED6, a NCED7, a NCED8, and a NCED9.

105. The transgenic plant of claim 104, wherein the promoter is a seed-specific promoter.

106. The transgenic plant of claim 105, wherein the expression the 9-cis-epoxycarotenoid dioxygenase (NCED) is enhanced.

107. The transgenic plant of claim 103, wherein the trans-acting factor comprises an ecdysone receptor and the cis-element comprises an ecdysone response element.

108. The transgenic plant of claim 103, wherein the positive chemical modulator is an ecdysone modulator.

109. The transgenic plant of claim 108, wherein the positive chemical modulator is selected from among a methoxyfenozide, a tubefenozide, and a diacylhydrazine.

110. The genetically modified plant of claim 103, which is a seed crop plant.

111. A genetically modified plant exhibiting controllable seed germination, comprising within its genome, and expressing, a heterologous polynucleotide encoding a 9-cis-epoxycarotenoid dioxygenase (NCED) selected from among a NCED6 and a NCED9 and a heterologous polynucleotide encoding an ecdysone receptor, wherein the polynucleotide encoding the 9-cis-epoxycarotenoid dioxygenase (NCED) is operably linked for expression to a promoter comprising an ecdysone response element that is regulated by the ecdysone receptor in the presence of a positive chemical modulator of the ecdysone receptor.

112. The genetically modified plant of claim 111, wherein the promoter is a seed-specific promoter.

113. The genetically modified plant of claim 112, wherein the expression NCED6 or the expression of NCED9 is enhanced.

114. The genetically modified plant of claim 111, wherein the positive chemical modulator of the ecdysone receptor is selected from among a methoxyfenozide, a tubefenozide, and a diacylhydrazine.

115. The genetically modified plant of claim 111, which is a seed crop plant.

116. A method of controlling seed germination, comprising administering a positive ecdysone modulator to the genetically modified plant of claim 106.

117. The method of claim 115, wherein the positive ecdysone modulator is selected from among a methoxyfenozide, a tubefenozide, and a diacylhydrazine.

118. The method of claim 115, wherein seed germination is suppressed.

119. The method of claim 117, wherein seed germination is recovered by administering an abscisic acid inhibitor.


SEQUENCE LISTING (37 sequences)

SEQ ID NO: 1 - DNA, 1734 bp, Arabidopsis thaliana
atgcaacactctcttcgttctgatcttcttccgacgaagacttctcctcgttctcatttacttccacaacccaaaaatgcaaatatttctcgacgaattctcattaaccctttcaagataccgacacttcctgatctcacttctccggttccgtcaccggttaagctcaaaccaacgtatccaaacttaaaccttcttcagaagctagcggctacgatgctcgacaagattgagtcctctatcgttattcctatggagcagaatcgcccgcttcctaaaccgaccgacccggcggttcaattatcaggtaacttcgctccggttaatgaatgtccggttcagaacggtttagaagtggttggtcagattccttcttgtctaaaaggagtttacatccgtaacggtgcaaaccctatgtttccgccgttagccggacatcatttatttgacggtgacggaatgattcacgccgttagtatcggttttgataaccaggttagttacagctgccggtacactaaaacaaaccggcttgttcaagaaaccgcgcttggacgatcggttttccctaaaccaatcggcgagcttcacggccattccggtctagctcgactcgctctcttcacggctcgagctgggatcggtctagtggacgggacacgtggcatgggcgtagctaacgccggtgtggttttctttaacggcaggttattagccatgtcagaagatgatcttccttaccaagtgaagatcgacggtcaaggagatcttgagacgatcggacggttcggattcgatgaccagattgactcttcagtgatagcgcatcctaaggtggacgcgaccacaggagatctccatacactgagctacaacgttttgaagaaacctcatctcaggtatcttaaattcaacacgtgcgggaaaaagacacgtgacgtggagatcacgctccctgaaccaacgatgattcatgatttcgcgataaccgagaattttgtcgttataccggatcagcaaatggtattcaaattatccgaaatgattcggggcgggtcacccgttatctacgttaaagaaaaaatggcgagatttggagttttgtcaaagcaggatctgaccgggtcggatataaattgggttgatgtaccggattgtttctgtttccatctatggaatgcgtgggaagagagaaccgaagagggagacccagttatcgtcgtaatcgggtcatgtatgagcccacccgacacgatctttagtgaatcaggagaaccaacccgggttgaattaagtgagatccggttaaacatgcgtacaaaagaatcgaaccgtaaggttatcgtaaccggagtgaatttagaagcgggtcacataaaccgtagttacgtgggccggaaaagccagttcgtttacatagcaatagccgatccttggcccaaatgcagtggcattgcgaaggtagatatacaaaacggcaccgtttcagagtttaattacggaccgagccggttcggtggagaaccgtgctttgtaccggagggagaaggagaagaagacaaaggttatgtaatggggtttgtgagagacgaagagaaagacgagtcggagtttgtggtggtcgacgcgacggatatgaagcaagtcgcggcggtgcgcttgccggagagggtaccttatggtttccatggaacgttcgtgagcgagaatcagttgaaggaacaagttttctga

SEQ ID NO: 2 - DNA, 1974 bp, Arabidopsis thaliana
atgacgataataaccattatttctggtatgtatatatatagccttctgtcccaagatgctcatcactcacaatatggacaaaacacaaacttggtcttgaagaaaccaataccaaaaccccaaaccgcagcgtttaatcaagaatcgactatggcttctactaccttacttccttctacttcaacccaattcctcgaccgaactttctcaacttcatcttcctcttcgagacctaaacttcaatctctatctttttcctcaacacttagaaacaagaaactagtagtcccctgctatgtttcttcctccgtcaacaaaaaatcttccgtctcgtcttctctccaatctccgacattcaaaccaccgtcttggaagaaactatgtaacgatgtcaccaacttgatccccaaaacgacgaatcagaatccaaaattaaacccggtgcagagaaccgccgctatggttttagacgcggtggaaaacgccatgatctcacacgagcgccgccgtcatccccatcctaaaacggcggatcctgccgtccaaatagccgggaacttcttcccggtaccggagaaacctgtcgtgcataaccttccggtgactggaacagtaccagaatgcattcaaggagtttacgtcagaaacggagcaaatccacttcacaaaccagtctccggccaccatttgttcgacggagacggtatggtacacgctgtccggttcgataacggctcggttagctacgcttgtcggtttaccgaaacaaaccggttggttcaagaacgtgaatgtggtcgtccggttttccccaaagcaattggagagcttcacggacatttaggaatcgccaagcttatgctcttcaatacccgcgggctatttgggttagtcgacccgaccggaggactcggtgtcgctaacgccggtttagtttatttcaatggtcatctcttagccatgtccgaagacgatttaccgtaccatgtcaaagttactcaaaccggagatttagaaacttcgggtcggtacgatttcgacggtcagttaaaatcaacaatgatagcccacccgaaaatcgatcccgaaacccgagaactcttcgctttaagctacgacgtcgtttcaaagccttacttaaaatacttcagattcacatccgacggtgagaaatcaccagacgtcgagattccgcttgatcaaccaacgatgatccacgatttcgcgatcactgagaatttcgtagtgattccagaccaacaagtggtattccgattgccggagatgataagaggtggttctccggtggtttacgacgagaagaagaaatcaagattcgggattttgaataaaaacgcgaaagatgcttcgtcgattcaatggatcgaagtaccagattgtttttgtttccatctatggaactcttgggaagaaccagaaacagacgaggttgtcgtgattggatcatgcatgacgccacctgattcaattttcaacgaacacgacgaaacacttcagagtgttttgtcggagataaggctaaaccttaaaacaggggaatcaacacgtagaccggttatctcggaacaagttaatctcgaagctggtatggtcaaccggaatttattaggtagaaaaacccggtatgcttatctggctttaaccgaaccgtggcctaaagtgtccggtttcgctaaagtggacttatcgaccggagagattcgaaagtatatttacggagaggggaaatacggaggagagcctctgtttctaccttccggtgacggagaagaagacggaggttacataatggtgttcgttcacgacgaggagaaggtgaaatcggaactccaactcataaacgccgttaatatgaagcttgaagctaccgtaacgcttccgtcgagagtgccttacggtttccacggaacgttcatcagtaaggaagatttatcgaagcaagctttgtgttag

SEQ ID NO: 3 - DNA, 1449 bp, Arabidopsis thaliana
atgcaaatctcatcttcatcgtcttcaaatttcttctcttctctttatgctgatgaaccggcactaatcacattaacaattgttgtagtagtagtagtgttactatttaaatggtggttgcactggaaagagcaaagactacggctacctcctggctccatggggttgccttacatcggagagacactccgcctctacacagaaaatcccaattccttcttcgccactcgccaaaacaagtacggggatatattcaagacgcacatattaggatgtccatgtgtgatgataagtagtccagaggcggctcgaatggtgttagtgagcaaagctcacttgttcaagccaacttatcctccaagcaaagagcgtatgattggaccagaggctcttttcttccaccaaggtccataccattctacccttaagcggctggtccagtcttctttcatgccttctgctctcagaccaaccgtctctcacatcgagctccttgtcctccaaaccctttcctcttggacgtcccaaaagtccatcaacaccctcgaatacatgaaacgatatgcattcgatgtggcgatcatgtcagcgttcggggacaaagaggagcccactacgattgatgttattaagcttctctatcaacgtctcgaaaggggttacaactccatgcctctcgacctaccgggcacactttttcataagtccatgaaggcaagaatagaattaagcgaggaactaaggaaagtaatagagaagagaagagagaatgggagagaagaaggaggactattgggagtacttctgggagcaaaggatcaaaaacgcaacggcttaagtgattcacagattgctgacaacatcatcggtgttatattcgccgccaccgacaccaccgcttctgtcttaacttggcttctcaagtacttacacgaccaccccaatctcctccaagaagtctccagggagcaattcagcattcgacagaaaataaaaaaagaaaaccgaagaatctcatgggaagatacaagaaaaatgccactgaccactagggtgatacaagagacactaagagcagcaagtgtactgtcctttacatttagagaagcagtacaagacgtcgaatatgatggctacttgatcccaaagggttggaaggttcttcctcttttccggcgaatccatcactcctccgaattcttccccgatcctgaaaaattcgatccttctagattcgaggtggcaccaaaaccttacacgtacatgccattcggaaatggagtgcactcatgtccaggaagtgagctggctaaacttgagatgcttatcctccttcaccacctcactacttccttcagatgggaagtgattggagatgaagaaggtatacagtatggtcctttccctgtacccaagaagggtttaccaataagagtaaccccgatttaa

SEQ ID NO: 4 - DNA, 1077 bp, Arabidopsis thaliana
atgcctgctatgttaacagatgtgtttagaggccatcccattcacctcccacactctcacatacctgacttcacatctctccgggagctcccggattcttacaagtggacccctaaagacgatctcctcttctccgctgctccttctcctccggccaccggtgaaaacatccctctcatcgacctcgaccacccggacgcgactaaccaaatcggtcatgcatgtagaacttggggtgccttccaaatctcaaaccacggcgtgcctttgggacttctccaagacattgagtttctcaccggtagtctcttcgggctacctgtccaacgcaagcttaagtctgctcggtcggagacaggtgtgtccggctacggcgtcgctcgtatcgcatctttcttcaataagcaaatgtggtccgaaggtttcaccatcactggctcgcctctcaacgatttccgtaaactttggccccaacatcacctcaactactgcgatatcgttgaagagtacgaggaacatatgaaaaagttggcatcgaaattgatgtggttagcactaaattcacttggggtcagcgaagaagacattgaatgggccagtctcagttcagatttaaactgggcccaagctgctctccagctaaatcactacccggtttgtcctgaaccggaccgagccatgggtctagcagctcataccgactccaccctcctaaccattctgtaccagaacaataccgccggtctacaagtatttcgcgatgatcttggttgggtcaccgtgccaccgtttcctggctcgctcgtggttaacgttggtgacctcttccacatcctatccaatggattgtttaaaagcgtgttgcaccgcgctcgggttaaccaaaccagagcccggttatctgtagcattcctttggggtccgcaatctgatatcaagatatcacctgtaccgaagctggttagtcccgttgaatcgcctctataccaatcggtgacatggaaagagtatcttcgaacaaaagcaactcacttcaacaaagctctttcaatgattagaaatcacagagaagaatga

SEQ ID NO: 5 - DNA, 12196 bp, Artificial Sequence
  Synthesized
gcggataacaatttcacacaggaaacagctatgaccatgattacgaattcgagctcggtaccccaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctgctagcaagacgtccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttggatccgccaccatgggccacaggcgcgcccactccgagatcctgagcctccccgaggacctcgacctgtgcgcggccggcggcggcgacgggccgtcgctgtcggacgagaacgacgaggagctcttctccatgttcctcgacgtggagaagctgaacagcacgtgcggggcgtcgtcggaggcggaggcggagtcgtcgtccgccgccgccgaattcggcgggatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggcgggtctagaagaatatcaaggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggagacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtctgacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtgttctgttcgcgaacaaccaagcgtacactcgcgacaactaccgcaaggctggcatggcctacgtcatcgaggatctactgcacttctgccggtgcatgtactctatggcgttggacaacatccattacgcgctgctcacggctgtcgtcatcttttctgaccggccagggttggagcagccgcaactggtggaagaaatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctagtaaccagtgtgctggatctacgcgttaagcttaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcactaggataaattatcgcgcgcggtgtcatctatgttactagatcggggactggggatcctctagagtcgaggccgcttggatccgctcggaggacagtactccgctcggaggacagtactccgctcgaggacagtactccgctcgaggacagtactccgctcggaggacagtactccgatccgtagatctgcaagacccttcctctatataaggaagttcatttcatttggagaggacacgctgaacgctggtgaccacgtcgtggaatgccttcgaattcagcacctgcacatgggacgtcgacctgaggtaattataacccgggccctatatatggatccaattgcaatgatcatcatgacagatctgcgcgcgatcgatatcagcgctttaaatttgcgcatgctagctatagttctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctgcagggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtga

SEQ ID NO: 6 - DNA, 1749 bp, Camelina Sativa
atgcaacactctcttcgttctgatcttcttcgttcgaagacttcttcttctcattctcatttacttccacaaaccaaaaatgtaaacatctcccgaagagttcttatcaaccctttcaagataacaacacttcctgatctccctcctctcttcaaatctccggtcacgccaccaccggttaagctcaaaccaacccatccaaacttaaaccctctccagaagctagcggcttcgatgctcgacaagatagagtcctctatcgtcataccgatggagcagaatcgaccgcttcctaaaccgaccgatccagcggttcaattatcaggtaacttcgctccggttcaagaatgtccggttcaaaacggtttagaagtcgttggtcagattccttcttgtctacgaggagtttacgtacgtaacggcgctaaccctatgtttccaccgttagccggacatcatttgtttgacggtgacggaatgattcacgccgttagtatcggttctgataacaaggttagttacagctgtcggtacactaaaacgaaccggcttgttgaagaaaccaagcttggaagatcggtttttcctaaaccaatcggcgagcttcacggccattccggtctagctcgactcgctctcttcgccgctcgagctgggataggtctcgtggacgggacacgtggcatgggtgtagccaacgccggtcttgtcttcttcaacgggaggttattagccatgtccgaagatgatctcccttaccaagtgaagatcgacggtcaaggagacctcgagacgatcggacggttcgggttcgacgatcaggtcgacttctcagtgatagcgcatcctaaggtggacgctaccacaggtgatctgcacacgctgagctacaacgttttgaggaaacctcatctcaggtatcttaaattcgacacttgcgggaaaaagacacgtgacgtggacatcacgctcccggaacctacgatgattcatgatttcgccataaccgagaattttgtcgtcataccggatcagcaaatggtattcaaattaaccgaaatgattcggggcgggtcaccggttatctacgataaagagaaaatgtcgagatttggggttttgtcaaagcaggatcagaccgggtcgggtataaattgggtcgacgtaccggattgtttctgtttccatctatggaacgcgtgggaagagagaaccgaagatggagatccggttattgtcgtactcgggtcgtgtatgagtccacccgacacgatcttcagtgaatcaggagaaccaacccggattgagttaagtgagatccggttaaacatgcggacaaaagaatcgaaccggagggttattgtaaccgggataaatttagaagcgggtcaaataaaccgcagtttcgtgggacggcaaaaccggtttgtttacttggcaatagctgatccttggcctaaatgcagtggcatcgctaaggtggatattgaaaacggcaccgttacaaagtttaattacggaccaggccggtttggtggcgaaccgtattttgtaccagaaaaggatggagaagacgaaggttatgtaatggggtttgttacggacgaagaaagaaacgagtcggagtttgtcgtggttgatgcaacggatatgaagcaagtcgccttggtgcgtctgccggagagggtaccttacggtttccatggaacgttcgtgagcgagaatcagttgaaagaacaagtttcctga

SEQ ID NO: 7 - protein, 482 aa, Arabidopsis thaliana
MQISSSSSSNFFSSLYADEPALITLTIVVVVVVLLFKWWLHWKEQRLRLPPGSMGLPYIGETLRLYTENPNSFFATRQNKYGDIFKTHILGCPCVMISSPEAARMVLVSKAHLFKPTYPPSKERMIGPEALFFHQGPYHSTLKRLVQSSFMPSALRPTVSHIELLVLQTLSSWTSQKSINTLEYMKRYAFDVAIMSAFGDKEEPTTIDVIKLLYQRLERGYNSMPLDLPGTLFHKSMKARIELSEELRKVIEKRRENGREEGGLLGVLLGAKDQKRNGLSDSQIADNIIGVIFAATDTTASVLTWLLKYLHDHPNLLQEVSREQFSIRQKIKKENRRISWEDTRKMPLTTRVIQETLRAASVLSFTFREAVQDVEYDGYLIPKGWKVLPLFRRIHHSSEFFPDPEKFDPSRFEVAPKPYTYMPFGNGVHSCPGSELAKLEMLILLHHLTTSFRWEVIGDEEGIQYGPFPVPKKGLPIRVTPI

SEQ ID NO: 8 - protein, 5 aa, Artificial Sequence
  Synthesized
DPAVQ

SEQ ID NO: 9 - protein, 7 aa, Artificial Sequence
  Synthesized
TNRLVQE

SEQ ID NO: 10 - protein, 11 aa, Artificial Sequence
  Synthesized
VPDCFCFHLWN

SEQ ID NO: 11 - protein, 3 aa, Artificial Sequence
  Synthesized
LLP

SEQ ID NO: 12 - protein, 3 aa, Artificial Sequence
  Synthesized
FDN

SEQ ID NO: 13 - protein, 3 aa, Artificial Sequence
  Synthesized
VSY

SEQ ID NO: 14 - protein, 4 aa, Artificial Sequence
  Synthesized
DEEK

SEQ ID NO: 15 - protein, 5 aa, Artificial Sequence
  Synthesized Xaa can be any naturally occuring amino acid
XXAVQ

SEQ ID NO: 16 - protein, 7 aa, Artificial Sequence
  Synthesized Xaa can be any naturally occurring amino acid
XNXLVQE

SEQ ID NO: 17 - protein, 11 aa, Artificial Sequence
  Synthesized Xaa can be any naturally occurring amino acid
VPDCXXXXXXX

SEQ ID NO: 18 - protein, 3 aa, Artificial Sequence
  Synthesized
NAN

SEQ ID NO: 19 - protein, 3 aa, Artificial Sequence
  Synthesized
PTY

SEQ ID NO: 20 - protein, 3 aa, Artificial Sequence
  Synthesized
QNG

SEQ ID NO: 21 - protein, 3 aa, Artificial Sequence
  Synthesized
DGQ

SEQ ID NO: 22 - protein, 4 aa, Artificial Sequence
  Synthesized
DLTG

SEQ ID NO: 23 - protein, 3 aa, Artificial Sequence
  Synthesized
SEF

SEQ ID NO: 24 - protein, 577 aa, Arabidopsis thaliana
MQHSLRSDLLPTKTSPRSHLLPQPKNANISRRILINPFKIPTLPDLTSPVPSPVKLKPTYPNLNLLQKLAATMLDKIESSIVIPMEQNRPLPKPTDPAVQLSGNFAPVNECPVQNGLEVVGQIPSCLKGVYIRNGANPMFPPLAGHHLFDGDGMIHAVSIGFDNQVSYSCRYTKTNRLVQETALGRSVFPKPIGELHGHSGLARLALFTARAGIGLVDGTRGMGVANAGVVFFNGRLLAMSEDDLPYQVKIDGQGDLETIGRFGFDDQIDSSVIAHPKVDATTGDLHTLSYNVLKKPHLRYLKFNTCGKKTRDVEITLPEPTMIHDFAITENFVVIPDQQMVFKLSEMIRGGSPVIYVKEKMARFGVLSKQDLTGSDINWVDVPDCFCFHLWNAWEERTEEGDPVIVVIGSCMSPPDTIFSESGEPTRVELSEIRLNMRTKESNRKVIVTGVNLEAGHINRSYVGRKSQFVYIAIADPWPKCSGIAKVDIQNGTVSEFNYGPSRFGGEPCFVPEGEGEEDKGYVMGFVRDEEKDESEFVVVDATDMKQVAAVRLPERVPYGFHGTFVSENQLKEQVF

SEQ ID NO: 25 - protein, 657 aa, Arabidopsis thaliana
MTIITIISGMYIYSLLSQDAHHSQYGQNTNLVLKKPIPKPQTAAFNQESTMASTTLLPSTSTQFLDRTFSTSSSSSRPKLQSLSFSSTLRNKKLVVPCYVSSSVNKKSSVSSSLQSPTFKPPSWKKLCNDVTNLIPKTTNQNPKLNPVQRTAAMVLDAVENAMISHERRRHPHPKTADPAVQIAGNFFPVPEKPVVHNLPVTGTVPECIQGVYVRNGANPLHKPVSGHHLFDGDGMVHAVRFDNGSVSYACRFTETNRLVQERECGRPVFPKAIGELHGHLGIAKLMLFNTRGLFGLVDPTGGLGVANAGLVYFNGHLLAMSEDDLPYHVKVTQTGDLETSGRYDFDGQLKSTMIAHPKIDPETRELFALSYDVVSKPYLKYFRFTSDGEKSPDVEIPLDQPTMIHDFAITENFVVIPDQQVVFRLPEMIRGGSPVVYDEKKKSRFGILNKNAKDASSIQWIEVPDCFCFHLWNSWEEPETDEVVVIGSCMTPPDSIFNEHDETLQSVLSEIRLNLKTGESTRRPVISEQVNLEAGMVNRNLLGRKTRYAYLALTEPWPKVSGFAKVDLSTGEIRKYIYGEGKYGGEPLFLPSGDGEEDGGYIMVFVHDEEKVKSELQLINAVNMKLEATVTLPSRVPYGFHGTFISKEDLSKQALC

SEQ ID NO: 26 - protein, 358 aa, Arabidopsis thaliana
MPAMLTDVFRGHPIHLPHSHIPDFTSLRELPDSYKWTPKDDLLFSAAPSPPATGENIPLIDLDHPDATNQIGHACRTWGAFQISNHGVPLGLLQDIEFLTGSLFGLPVQRKLKSARSETGVSGYGVARIASFFNKQMWSEGFTITGSPLNDFRKLWPQHHLNYCDIVEEYEEHMKKLASKLMWLALNSLGVSEEDIEWASLSSDLNWAQAALQLNHYPVCPEPDRAMGLAAHTDSTLLTILYQNNTAGLQVFRDDLGWVTVPPFPGSLVVNVGDLFHILSNGLFKSVLHRARVNQTRARLSVAFLWGPQSDIKISPVPKLVSPVESPLYQSVTWKEYLRTKATHFNKALSMIRNHREE

SEQ ID NO: 27 - protein, 87 aa, herpes simplex virus
KVAPPTDVSLGDELHLDGEDVAMAHADALDDFDLDMLGDGDSPGPGFTPHDSAPYGALDMADFEFEQMFTDALGIDEYGGEFPGIRR

SEQ ID NO: 28 - protein, 368 aa, Rice
MNREKSPIPGDGGDGLPPQATRRAGPPAAAAAAEYDISRMPDFPTRNPGHRRAHSEILSLPEDLDLCAAGGGDGPSLSDENDEELFSMFLDVEKLNSTCGASSEAEAESSSAAAHGARPKHQHSLSMDESMSIKAEELVGASPGTEGMSSAEAKKAVSAVKLAELALVDPKRAKRIWANRQSAARSKERKMRYIAELERKVQTLQTEATTLSAQLALLQRDTSGLTTENSELKLRLQTMEQQVHLQDALNDTLKSEVQRLKVATGQMANGGGMMMNFGGMPHQFGGNQQMFQNNQAMQSMLAAHQLQQLQLHPQAQQQQVLHPQHQQQQPLHPLQAQQLQQAARDLKMKSPMGGQSQWGDGKSGSSGN

SEQ ID NO: 29 - protein, 147 aa, UNKNOWN
  yeast
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 30 - protein, 541 aa, cloristoneura fumiferana
MRRRWSNNGGFQTLRMLEESSSEVTSSSALGLPAAMVMSPESLASPEYGGLELWGYDDGLSYNTAQSLLGNTCTMQQQQQTQPLPSMPLPMPPTTPKSENESISSGREELSPASSINGCSTDGEARRQKKGPAPRQQEELCLVCGDRASGYHYNALTCEGCKGFFRRSVTKNAVYICKFGHACEMDMYMRRKCQECRLKKCLAVGMRPECVVPETQCAMKRKEKKAQKEKDKLPVSTTTVDDHMPPIMQCEPPPPEAARIHEVVPRFLSDKLLETNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 31 - DNA, 17 bp, Artificial Sequence
  Synthesized
cggrnnrcynyncnccg

SEQ ID NO: 32 - DNA, 20 bp, Artificial Sequence
  Synthesized
cataggtcgctcacaagtca

SEQ ID NO: 33 - DNA, 21 bp, Artificial Sequence
  Synthesized
acgaagagagtgttgcatggt

SEQ ID NO: 34 - DNA, 21 bp, Artificial Sequence
  Synthesized
cgaatgtctcacatcgttggt

SEQ ID NO: 35 - DNA, 20 bp, Artificial Sequence
  Synthesized
aggtctcgaagaggaagatg

SEQ ID NO: 36 - DNA, 2041 bp, Arabidopsis thaliana
tcgagagatcgatccgacttttcaaatcagttcttcatcaatagacgtatgtctcttacatataatcaatcagatgacccaaaaaaaactatttgaaatagtgaataaaagttaaaaattatttcaaatacttcatacaaatataataaaaataaaaataaaatctaattaaatctataaaattgtagttttaagataccattgtattaaatggacatatctccttattccgagagccagattgaagacaaatttgtaaatgaaaatggtcgatcaaagataaaattttagaatggccactagcattaaagtaaattagtggtttgatatataaactttgtttagcacactaccaccatcgacttcgaatctccgcaatattgccaccaccaccttccaatctccgcattgttgcatttttcattatttaatgctgtaaaacaaaactaaactgtgaatcaaaataaagcataccaaaattttctgacttggcggcgtctaataaccaatccaatccgattcaagaaacttggactctaagcgaccctaacaaacggtccactaccatctccccagtgtattccactagcaacttcacattattcatgacacatttaatcacaataattgattagtaacttttggggactaaatttagatgtattatccttactagtactaaacgtgaaagttttatcacacaccgcatgaattttacattatagttaaaacacacacacctgccacaaggtcataccaattatattacgaatataaaaaagtctttccactgttccacataatcttccttctttatcttccacaatatctcattcaccagcggttaccaaaagaaaaaaaaaaaactattctcattcactatgcacattattgtggcaaaacaaaatctaattcaccaacggttacacaactctatgccaaactttgtttttcgaaattctgtattctgaaaacattcgactaattttgtacaaaaacacatgtgtacacgggcaaaaaaagcacatatctattattttaaaaaagattaacagattatcgttcttccattactttgtaatgaatgactaattttttttactaaacttttatattaaacctgtgtctaattttatgactaatgaatttcttttgactaatatttaattattgaacacatgtgttatactcattggcaataaaaacacacatagatgtctttagttaataaaataaattataaaaatgttattcaaaagaatattattggtcttccaatatacactttctttggatttactatgaaacagacaatcttcataaaatttaaagaaatctattaaaaacaaaacttcaaattacagcagaccctataataattgtaaggattcacttctatatatgtgcctgattaataaaaggtaaaaaatcttatagttttctaaatttataattactacttttgggagatttgatacctatttagaattcactctcaaacaaaaaattcccaccgttgaaacaaaaagtaaatgtgtgtgaaccaatcaaaatcgttgaattaaggaaaaatctccacattatagcataccttataatctggatattttctgctatatatgtctctaataaagtaaaattgtaaaaagaacaaaatattacagttgttattcctatacgtatacgtaataactatttttggaagataagattcgacacctatttaggttcacttttaaaccaaaaaaaatctctacccttgaaacaaagaataaatataagtaaaacaatcagaatcgttggattaaaataaatctacaaaatactccacattatagtagagaacatataatctagatttgctattaaatatggtatcttgacaaatgtcaagaccccgttacattttctatcatctttatataaaccgctagcttttaccctcttgttttgggcaaacaccaaaaacgtttttacttataaaacgttagaaaacttctctagtcatgttttatgcataggtcgctcacaagtcaccacc

SEQ ID NO: 37 - DNA, 300 bp, Arabidopsis thaliana
ctggatctacgcgttaagcttaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcactaggataaattatcgcgcgcggtgtcatctatgttactagatcggggactggggatcctctaga